(19) United States
(12) Patent Application Publication
(10) Pub. No.: US 2026/0241021 A1
(43) Pub. Date: Aug. 20, 2026

(54) IMPROVED LINKER-PAYLOADS FOR ANTIBODY CONJUGATION, PHARMACEUTICAL COMPOSITIONS AND APPLICATIONS THEREOF

(71) Applicant: OBI PHARMA, INC., Taipei City (TW)

(72) Inventors: Ming-Tain Lai, Taipei City (TW); Hwei-Gene Wang, Taipei City (TW); Teng-Yi Huang, Taipei City (TW); Yu-Chao Huang, Taipei City (TW); Yu Lee, Taipei City (TW)

(73) Assignee: OBI PHARMA, INC., Taipei City (TW)

(21) Appl. No.: 19/164,584

(22) PCT Filed: Sep. 20, 2024
(86) PCT No.: PCT/US2024/047573

§ 371 Date: Sep. 12, 2025

Related U.S. Application Data
(60) Provisional application No. 63/584,559, filed on Sep. 22, 2023.
(60) Provisional application No. 63/659,345, filed on Jun. 13, 2024.

(51) Int. Cl. A61K 47/68; A61K 47/60; A61P 35/00; C07K 16/28; C07K 16/30; C07K 16/32

(52) U.S. Cl. CPC A61K 47/68037; A61K 47/60; A61K 47/6849; A61K 47/6855; A61K 47/6879; A61K 47/6889; A61P 35/00; C07K 16/2863; C07K 16/30; C07K 16/32

(57) ABSTRACT

The present disclosure provides linker-payload conjugates including a bioorthogonal group and a hydrophilic moiety. The improved linker-payloads for antibody conjugation and improved antibody drug conjugates (ADCs) exhibit greater stability in blood circulation and enhanced drug delivery and drug release efficiencies in target cells. The present disclosure also relates to antibodies and antigen-binding fragments thereof for several antigen targets (e.g. TROP2, HER2, Nectin-4, etc.), as well as pharmaceutical compositions including ADCs. Also described herein are methods of using ADCs for treatment of subjects associated with pathological conditions.

### CROSS-REFERENCE TO RELATED APPLICATIONS

This application claims the priority of U.S. Provisional Patent Application Nos. 63/584,559 (filed on Sep. 22, 2023) and 63/659,345 (filed on Jun. 13, 2024). The content of the aforementioned applications is incorporated herein in its entirety by reference.

### FIELD

The present disclosure relates to linker-payloads, antibody conjugates, and methods for treatment of diseases using the antibody conjugates.

### BACKGROUND OF THE INVENTION

A great deal of interest in cancer therapies is focused on the use of monoclonal antibodies (mAbs) for the targeted delivery of cytotoxic agents to cancer cells. The design of antibody-drug conjugates (ADCs), by attaching a drug to an antibody, typically via a linker, involves consideration of a variety of factors. These factors include the types of antibodies and linkers for the conjugation of drugs. Further, if the drug is to be released after antibody internalization, the mechanism of drug release, and the associated structural elements, and the structural modification of the drug after release, if any, also need to be taken into account. For example, the structural elements and mechanisms of drug release must be consistent with the intracellular trafficking of the conjugate.

The antibodies for ADC construction can be small protein formats (e.g., scFv or Fab fragments, designed ankyrin repeat proteins, affibodies, nanobody, etc.), monoclonal antibodies (mAbs), and bispecific antibodies which have been selected based on their high selectivity and affinity for a given antigen, their long circulating half-lives, and little to no immunogenicity. Thus, mAbs as protein ligands for a carefully selected biological receptor provide an ideal platform for the delivery of drugs to target cells. For example, a monoclonal antibody known to bind selectively with a tumor-associated antigen can be used for delivery of a conjugated cytotoxic agent to the tumor through its target-specific binding to the antigen and subsequent internalization of the cytotoxic agent. The cytotoxic agent may be a small molecule toxin, a protein toxin, an agonist, an antagonist, or in other formats, like oligonucleotides. Hence, the concept of targeted drug delivery to a specific cellular location of choice is a powerful approach to treating a wide range of diseases, with many beneficial aspects versus systemic delivery of the same drug.

In the field of ADCs, a chemical linker is typically employed to attach a drug to an antibody. This linker needs to possess several key attributes, including the requirement to be stable in blood during circulation after ADC administration for an extended period of time. A stable linker enables the localization of the ADC to the projected site or cells in the body and prevents the premature release of the drug in circulation, which would indiscriminately induce undesired biological responses of all kinds, thereby broadening the therapeutic index of the ADC. Upon internalization, the ADC is cleaved such that the drug is effectively released to kill the targeted cells.

ADCs can be prepared by conjugation of a linker-payload with an antibody. Two main technologies are recognized for the preparation of ADCs by random conjugation, either based on acylation of a lysine side chain or based on alkylation of a cysteine side chain in the antibody. Acylation of the amino group in a lysine side-chain is typically achieved by subjecting the antibody to a reagent with an activated ester or activated carbonate derivative, for example, succinimidyl-trans-4-(N-maleimidylmethyl)cyclohexane-1-carboxylate (SMCC) is applied for the manufacturing of Kadcyla®. The main chemistry for the alkylation of the thiol group in a cysteine sidechain is based on the use of maleimide reagents, as is applied in the manufacturing of Adcetris®. Besides standard maleimide derivatives, a range of maleimide variants are also applied for more stable cysteine conjugation (Lyon et al., Nat. Biotechnol. 2014, 32, 1059-1062).

Although recent clinical trials have demonstrated the efficacy of ADCs in cancer therapy, several challenges remain. One of these is the off-target side effects caused by premature drug release into the bloodstream, which negatively affects the therapeutic index of ADCs. Therefore, there is an urgent need to develop new linker-payloads to generate ADCs with improved stability and efficient drug release that is well-controlled.

### SUMMARY OF THE INVENTION

The inventors found that cleavable linker-payloads are highly suitable for metal-free click conjugation of drugs to antibodies and that attaching specific hydrophilic moieties to said linker-payloads caused the resulting ADCs to display improved in vitro and in vivo cytotoxicity and antitumor activity. Accordingly, the present disclosure provides improved linker-payloads that may be used in conjugation with antibodies, such as glycan-engineered antibodies, as well as improved antibody conjugates.

In one aspect, the present disclosure provides a linker-payload of Formula (III):

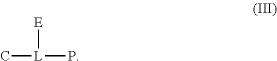

The linker-payload includes a bioorthogonal group (represented as C) for conjugation to an antibody or an antigen-binding fragment thereof; a linker unit (represented as L) that is covalently linked to the bioorthogonal group and includes a cleavable moiety, for example, a protease-cleavable peptide moiety; a hydrophilic moiety (represented as E) attached covalently to the linker unit; and a payload (represented as P) selected from a drug unit or a probe unit that is covalently linked to the linker unit. The hydrophilic moiety may be a linear or non-linear hydrophilic polymer, such as polyethylene glycol (PEG), and may be extended to a suitable length with various modifications on the hydrophilic polymer.

In certain embodiments, the drug unit may be a chemotherapeutic agent, a toxin, a cytokine, a growth inhibitory agent, a protein degrader, a peptide, a radionuclide, a hormone, an anti-viral agent, an anti-bacterial agent, or an immunoregulatory agent. In certain embodiments, the chemotherapeutic agent is a topoisomerase inhibitor, including a topoisomerase I inhibitor and a topoisomerase II inhibitor. The topoisomerase I inhibitors include but are not limited to camptothecin (CPT) compounds and non-camptothecin compound, for example, exatecan. In certain embodiments, the probe unit may be an imaging agent, such as a fluorophore, a dye, a contrast agent, or a radionuclide.

In another aspect, the present disclosure provides an antibody conjugate, such as an antibody-drug conjugate (ADC) or an antibody-probe conjugate, prepared by conjugating the linker-payload disclosed herein with an antibody or an antigen-binding fragment thereof. The ADCs disclosed herein exhibit greater stability in blood circulation, enhancing drug delivery and the subsequent drug release efficiencies in target tumor cells, thus improving therapeutic index of the ADCs with lower off-target toxicity. The enhanced stability of the ADCs in the body may be attributed to the hydrophilic moiety (e.g., PEG), which, at an appropriate length and an appropriate distance from the payload, can effectively protect the cleavable linker-payloads.

In certain embodiments, the antibody is a monoclonal antibody. The antibody or the antigen-binding fragment thereof may be monospecific, such as an antibody specific to CLDN18.2, B7-H3, MUC-1, PD-1, PD-L1, CTLA-4, VEGF, BCMA, PSMA, CGRP, Tfr1, TNF alpha, tissue factor, folate receptor alpha, carcinoembryonic antigens (CEACAMs), c-MET, HER3, EGFR, HER2, TROP2, or Nectin-4. Alternatively, the antibody or the antigen-binding fragment thereof may be multispecific, such as an anti-HER2 and anti-TROP2 bispecific antibody, an anti-c-Met and anti-HER3 bispecific antibody, an anti-EGFR and anti-HER3 bispecific antibody, or an anti-EGFR and anti-c-Met bispecific antibody. In certain embodiments, the antibody is an anti-TROP2 antibody selected from hRS7, Hu2G10, hu4D3, MAAP-9001a, Pr1E11, R4702, datopotamb, or sacituzumab. In certain preferred embodiment, the antibody is R4702. R4702 is as described in PCT patent publication No. WO2022222992A1, the contents of which is incorporated herein by reference in its entirety.

In certain embodiments, the antibody is a glycan-engineered antibody, including an N-linked glycan at an asparagine residue of the antibody (e.g., the N297 of a human IgG heavy chain constant region) that is modified through deglycosylation and transglycosylation to have a defined glycan structure for drug conjugation. The deglycosylation and transglycosylation may be accomplished using glycosynthases and variants thereof. In certain embodiments, the glycosynthase variants are EndoSd-D232M and EndoSz-D234M. Exemplary EndoSd-D232M and EndoSz-D234M are as described in PCT patent publication WO2020006176A1.

In another aspect, the present disclosure provides a method for preparing a glycan-engineered antibody conjugate, including reacting a glycan-engineered antibody with the linker-payload disclosed herein, wherein the glycan-engineered antibody includes a fucosylated or non-fucosylated N-acetylglucosamine (GlcNAc) at an asparagine residue (e.g., the N297 of human IgG heavy chain constant region) coupled to a glycan. The glycan-engineered antibody may be obtained by contacting an antibody with a glycosynthase and a glycan oxazoline to couple the glycan with the fucosylated or non-fucosylated GlcNAc. This method offers a unique glycan-engineered antibody conjugate (e.g., a glycan-engineered ADC) platform that is distinct from conventional ones by high product homogeneity. Therefore, site-specific ADCs with homogeneous DAR (drug-to-antibody ratio) can be prepared readily. These glycan-engineered ADCs have favorable manufacturing, quality control, and in vivo pharmacokinetic profiles.

In another aspect, the present disclosure provides a pharmaceutical composition, including the ADC described herein and a pharmaceutically acceptable carrier.

In another aspect, the present disclosure provides a method for inhibiting proliferation of cancer cells, including contacting with the cancer cells with an effective amount of the ADC described herein.

In another aspect, the present disclosure provides a method for treating a disease such as cancer, infection, nervous system, inflammatory, or autoimmune disorders, including administering to a subject in need thereof an effective amount of the ADC described herein. The effective amount will vary depending on various factors, including but not limited to the type of the ADC, the physiological conditions (e.g., general health or age) of a subject, the type and severity of the cancer, the treatment regimens, and the presence of other diseases. Generally, the effective amount is in the range of 0.01 μg-250 mg per kilogram body weight of a human subject.

In certain embodiments, the disease is characterized by expressing CLDN18.2, B7-H3, MUC-1, PD-1, PD-L1, CTLA-4, VEGF, BCMA, PSMA, CGRP, Tfr1, TNF alpha, tissue factor, folate receptor alpha, carcinoembryonic antigens (CEACAMs), c-MET, HER3, EGFR, HER2, TROP2, or Nectin-4. In certain embodiments, the disease is a cancer selected from the group consisting of multiple myeloma, acute myeloid leukemia (AML), sarcoma, skin cancer, leukemia, lymphoma, brain cancer, glioblastoma, lung cancer, breast cancer, head-and-neck cancer, nasopharyngeal cancer, esophagus cancer, stomach cancer, liver cancer, bile duct cancer, gallbladder cancer, bladder cancer, pancreatic cancer, intestinal cancer, colorectal cancer, kidney cancer, cervix cancer, endometrial cancer, ovarian cancer, testicular cancer, buccal cancer, oropharyngeal cancer, laryngeal cancer, prostate cancer, thyroid cancer, and oral cancer.

In another aspect, the present disclosure provides a method for preparing an ADC, including reacting an antibody or an antigen-binding fragment thereof with the linker-payload disclosed herein to obtain the ADC, wherein the payload is the drug unit.

The details of one or more embodiments of the invention are set forth in the description below. The features or advantages of the present invention will be apparent from the detailed description of preferred embodiments and the accompanying drawings.

### BRIEF DESCRIPTION OF THE DRAWINGS

FIGS. 1A-1F show the SDS-PAGE and CE-SDS results verifying mAb-(NSCT-di-N3)₂ production; FIG. 1A shows the SDS-PAGE analysis of R4702 antibody (anti-TROP2 mAb) and its glycan-engineered products; FIG. 1B shows the SDS-PAGE analysis of TX05 antibody (anti-HER2 mAb) and its glycan-engineered products; FIGS. 1C-IF show the CE-SDS electropherograms for the indicated glycan-engineered antibodies.

FIGS. 2A-2D show the mass spectra of two exemplary ADCs incubated with human serum albumin (HSA) for 0 or 6 days; FIGS. 2A-2B show the spectra of R4702-MCCA-PEG₂₄-Exatecan ADC (referred to as ADC-1) on Day 0 and Day 6, respectively; FIGS. 2C-2D show the spectra of R4702-DBCO-PEG₂₄-Exatecan ADC (referred to as ADC-2) on Day 0 and Day 6, respectively. LC0, LC1, HC0, HC1, HC2, and HC3 denote the signals of the light chain (LC) and the heavy chain (HC) of the indicated ADCs with 0, 1, 2, or 3 payloads, respectively; FIG. 2E shows the DAR change profiles of ADC-1 and ADC-2.

FIG. 3 shows the payload release profiles over time of ADC-1 and ADC-2 in human plasma.

FIG. 4A shows the cytotoxicity of ADC-1 and ADC-2 in human lung cancer NCI-H1975-C797S cells; FIG. 4B shows the cytotoxicity of ADC-1 and ADC-2 in human prostate cancer DU-145 cells; FIG. 4C shows the cytotoxicity of TX05-MCCA-PEG₂₄-Exatecan ADC (referred to as ADC-3) and TX05-DBCO-PEG₂₄-Exatecan ADC (referred to as ADC-4) in human gastric cancer NCI-N87 cells; FIG. 4D shows the cytotoxicity of ADC-3 and ADC-4 in human pancreatic cancer Capan-1 cells.

FIGS. 5A-5B respectively show the changes in tumor volume and body weight of NCI-H1975-C797S human lung cancer xenograft mice treated with ADC-1 and ADC-2.

FIGS. 6A-6E show the chromatograms of R4702 ADCs with varying PEG lengths, analyzed by hydrophilic interaction chromatography (HIC), wherein FIG. 6A shows the chromatogram of R4702-BCN-PEG₄₈-Exatecan ADC (referred to as ADC-5), FIG. 6B shows the chromatogram of R4702-BCN-PEG₂₄-Exatecan ADC (referred to as ADC-6), FIG. 6C shows the chromatogram of R4702-BCN-PEG₁₂-Exatecan ADC (referred to as ADC-7), FIG. 6D shows the chromatogram of R4702-BCN-PEG₆-Exatecan ADC (referred to as ADC-8), and FIG. 6E shows the chromatogram of R4702-BCN-Exatecan ADC (referred to as ADC-9).

FIGS. 7A-7C respectively show the cytotoxicity of R4702 ADCs with varying PEG lengths, assessed by 3D cytotoxicity assay, in three human cancer cells: NCI-N87 sh VOID (with high TROP2 expression), NCI-N87 shTROP2 (with low TROP2 expression), and NCI-H1975 (with medium TROP2 expression).

FIG. 8 shows the payload release efficiency of R4702 ADCs with different PEG lengths.

FIGS. 9A-9B respectively show the changes in tumor volume and body weight of NCI-H1975 human non-small cell lung cancer xenograft mice treated with R4702 ADCs with varying PEG lengths.

FIG. 10A shows the digestion efficiency of exemplary linker-payloads; and FIG. 10B shows the exatecan release efficiency of exemplary linker-payloads.

FIG. 11 shows the cytotoxicity of exemplary linker-payloads in human breast cancer cells SKBR-3.

FIG. 12 shows the payload release efficiency of R4702 ADCs with different bioorthogonal groups.

FIG. 13 shows the cytotoxicity of exemplary R4702 ADCs with different bioorthogonal groups in pancreatic adenocarcinoma cells BxPC-3.

### DETAILED DESCRIPTION OF THE PREFERRED EMBODIMENT

### Abbreviations

ACN: acetonitrile; ADC: Antibody-drug conjugate; ADCC: antibody-dependent cellular cytotoxicity; BCN: Bicyclononyne; bsADC: bispecific ADC; CDC: complement dependent cytotoxicity; CDR: complementarity-determining region; CE-SDS: capillary electrophoresis-sodium dodecyl sulfate; DAR: drug-to-antibody ratio; DBCO: Dibenzocyclooctyne; DL: drug-linker compound; DMSO: dimethyl sulfoxide; FA: formic acid; FUT8: α-1,6-fucosyltransferase 8; GlcNAc: N-acetylglycosamine; HC: heavy chain; HFIP: 1,1,1,3,3,3-Hexafluoro-2-propanol; HIC: hydrophilic interaction chromatography; HRMS: high resolution mass spectrometry; HSA: human serum albumin; IPTG: isopropyl-β-D-thiogalactopyranoside; ISTD: internal standard; LC: light chain; mAb: monoclonal antibody; MCCA: 4-(N-Maleimidomethyl)-cyclohexane-1-carboxylate; NaOAc: sodium acetate; NaOH: sodium hydroxide; NSCT: sialylated complex type N-glycan; PAB: Para-aminobenzyl alcohol; PBS: phosphate buffered saline; TFA: trifluoroacetic acid; T785:1-(4-aminobutyl)-2-butylimidazo[4,5-c]quinolin-4-amine; PSar20: polysarcosine 20; SMCC: succinimidyl-trans-4-(N-maleimidylmethyl)cyclohexane-1-carboxylate; MMAE: monomethyl auristatin E; SDS-PAGE: sodium dodecyl sulfate polyacrylamide gel electrophoresis.

### Definitions

As used herein, the singular forms “a”, “an”, and “the” include plural reference unless the context clearly dictates otherwise.

As used herein, the term “glycan” refers to a polysaccharide, oligosaccharide or monosaccharide. Glycans can be monomers or polymers of sugar residues and have a linear or branched structure. A glycan may include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′ sulfo N-acetylglucosamine, etc.).

As used herein, the terms “fucose”, “core fucose”, and “core fucose residue” are used interchangeably and refer to a fucose in α-1,6-position linked to the N-acetylglucosamine.

As used herein, the terms “N-glycan” and “N-linked glycan” are used interchangeably and refer to an N-linked oligosaccharide attached via an N-acetylglucosamine (GlcNAc) to the amide nitrogen of an asparagine residue in a protein or an antibody.

As used herein, the terms “glycosylation pattern” and “glycosylation profile” are used interchangeably and refer to the characteristic “fingerprint” of the N-glycan species on a glycoprotein or antibody. The glycosylation profile can be obtained by collecting a N-glycan species released from a glycoprotein through enzymatic digestion or chemical hydrolysis, and then analyzing the carbohydrate structure, for example, LC-HPLC, or MALDI-TOF MS, and the like.

As used herein, the term “antigen” is defined as any substance capable of eliciting an immune response.

As used herein, the term “epitope” is defined as the parts of an antigen molecule which contact the antigen binding site of an antibody or a T cell receptor.

As used herein, the term “antigen specific” refers to a property of a cell population such that supply of a particular antigen, or a fragment of the antigen, results in specific cell proliferation.

As used herein, the term “specific binding” refers to the interaction between binding pairs (e.g., an antibody and an antigen). In various instances, specifically binding can be embodied by an affinity constant of about 10-6 mol/L, about 10-7 mol/L, or about 10-8 mol/L, or less.

The phrase “substantially similar,” “substantially the same”, “equivalent”, or “substantially equivalent”, as used herein, denotes a sufficiently high degree of similarity between two numeric values (for example, one associated with a molecule and the other associated with a reference/comparator molecule) such that one of skill in the art would consider the difference between the two values to be of little or no biological and/or statistical significance within the context of the biological characteristic measured by said values (e.g., Kd values, anti-viral effects, etc.). The difference between said two values is, for example, less than about 50%, less than about 40%, less than about 30%, less than about 20%, and/or less than about 10% as a function of the value for the reference/comparator molecule.

The terms “antibody” and “immunoglobulin” are used interchangeably in the broadest sense and include monoclonal antibodies (e.g., full-length or intact monoclonal antibodies), polyclonal antibodies, monovalent antibodies, multivalent antibodies, monospecific antibodies, multispecific antibodies (e.g., bispecific antibodies so long as they exhibit the desired biological activity) and sometimes may refer to antibody fragments, as described in greater detail herein. An antibody can be chimeric, human, humanized and/or affinity matured. An antibody can be a full-length or a fragment (or a combination of fragments) of an antibody having an antigen-binding portion according to the context. The fragment includes, but are not limited to, Fab, F(ab′)₂, Fab′, F(ab)′, Fv, single chain Fv (scFv), bivalent scFv (bi-scFv), trivalent scFv (tri-scFv), Fd, dAb fragment, an CDR, diabodies, triabodies, tetrabodies, nanobodies, single-chain antibody molecules, or multispecific antibodies formed from antibody fragments. Single-chain antibodies produced by encompassed by the present disclosure.

The antibody disclosed herein may include a heavy chain or light chain variable region, a heavy chain or light chain constant region, a framework region, or any portion thereof. The antibody or antigen-binding fragment thereof may be mammalian-derived, including murine and human antibodies.

The phrase “variable region” or “variable domain” of an antibody refers to the amino-terminal domains of heavy chains or light chain of the antibody. These domains are generally the most variable parts of an antibody and contain the antigen-binding sites. The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR).

The term “Fab” refers to two identical antigen-binding fragments produced by papain digestion of antibodies, each of which with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “Fv” refers to the minimum antibody fragment which contains a complete antigen-recognition and -binding site. In a two-chain Fv species, this region consists of a dimer of one heavy- and one light-chain variable domain in tight, non-covalent association. In a single-chain Fv species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The phrase “light chains” of antibodies from any vertebrate species can be assigned to one of two clearly distinct types, called kappa (κ) and lambda (λ), based on the amino acid sequences of their constant domains.

Depending the amino acid sequences of the constant domains of their heavy chains, antibodies (immunoglobulins) can be assigned to different classes. There are five major classes of immunoglobulins: IgA, IgD, IgE, IgG and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively. An antibody may be part of a larger fusion molecule, formed by covalent or non-covalent association of the antibody with one or more other proteins or peptides.

The phrases “full-length antibody,” “intact antibody” and “whole antibody” are used herein interchangeably, to refer to an antibody in its substantially intact form, not antibody fragments as defined below. The terms particularly refer to an antibody with heavy chains that contain the Fc region.

The phrase “antibody fragments” refers to a portion of an intact antibody, wherein the portion retains at least one, or as many as most or all, of the functions normally associated with that portion when present in an intact antibody. In one embodiment, an antibody fragment contains an antigen binding site of an intact antibody and thus retains the ability to bind antigen. In another embodiment, an antibody fragment includes a Fc region of an antibody and retains at least one of the biological functions normally associated with the Fc region when present in the intact antibody, such as FcRn binding, antibody half-life modulation, ADCC function and complement binding. In one embodiment, an antibody fragment is a monovalent antibody that has an in vivo half-life substantially similar to an intact antibody. For example, such an antibody fragment may contain an antigen binding arm linked to an Fc sequence capable of conferring in vivo stability to the fragment.

The phrase “monoclonal antibody” refers to an antibody obtained from a population of substantially homogeneous antibodies, i.e., the individual antibodies within the population are identical except for possible naturally occurring mutations that may be present in minor amounts. Thus, the modifier “monoclonal” indicates the character of the antibody as not being a mixture of discrete antibodies. Such monoclonal antibody typically includes an antibody including a polypeptide sequence that enables the antibody to bind a target, wherein the target-binding polypeptide sequence was obtained by a process that includes the selection of a single target-binding polypeptide sequence from a plurality of polypeptide sequences. For example, the selection process can be the selection of a unique clone from a plurality of clones, such as a pool of hybridoma clones, phage clones or recombinant DNA clones. It should be understood that the selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method.

The phrase “monoclonal antibodies” as used herein may include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

The antibodies or antigen-binding fragments thereof may be peptides. Such peptides can include variants, analogs, orthologs, homologs and derivatives of peptides, that exhibit a biological activity, e.g., binding of a carbohydrate antigen or a tumor associated antigen. The peptides may contain one or more analogs of amino acids (for example, non-naturally occurring amino acids, the amino acids only occurring naturally in non-mammals, and the amino acids modified from those present in mammals, etc.), substituted linkages, as well as other modifications known in the art.

The antibody or antigen-binding fragment thereof, can be derivatized or linked to other functional molecules. For example, an antibody can be functionally linked (by chemical coupling, genetic fusion, noncovalent interaction, etc.) to one or more other molecular entities, such as another antibody, a detectable agent, a cytotoxic agent, a pharmaceutical agent, a protein or peptide that can mediate association with another molecule (such as a streptavidin core region or a polyhistidine tag), amino acid linkers, signal sequences, immunogenic carriers, or ligands useful in protein purification, such as glutathione-S-transferase, histidine tag, and staphylococcal protein A. One type of derivatized protein is produced by crosslinking two or more proteins (of the same type or of different types). Suitable crosslinkers include those that are heterobifunctional, having two distinct reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, 111. Useful detectable agents with which a protein can be derivatized (or labeled) include fluorescent compounds, various enzymes, prosthetic groups, luminescent materials, bioluminescent materials, and radioactive materials. Non-limiting, exemplary fluorescent detectable agents include fluorescein, fluorescein isothiocyanate, rhodamine, and, phycoerythrin. A protein or antibody can also be derivatized with detectable enzymes, such as alkaline phosphatase, horseradish peroxidase, beta-galactosidase, acetylcholinesterase, glucose oxidase and the like. A protein can also be derivatized with a prosthetic group (e.g., streptavidin/biotin and avidin/biotin).

The term “humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region of the recipient are replaced by residues from a hypervariable region of a non-human species (donor antibody) such as mouse, rat, rabbit or nonhuman primate having the desired specificity, affinity, and/or capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin.

The term “hypervariable region”, “HVR”, or “HV”, when used herein refers to the regions of an antibody variable domain which are hypervariable in sequence and/or form structurally defined loops. Generally, antibodies comprise six hypervariable regions; three in the VH (VH1, VH2, VH3), and three in the VL (VL1, VL2, VL3). A number of hypervariable region delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)).

The term “framework” or “FW” residues refers to those variable domain residues other than the hypervariable region residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The term “single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

A “human antibody” is one which possesses an amino acid sequence which corresponds to that of an antibody produced by a human and/or has been made using any of the techniques for making human antibodies as disclosed herein.

A “blocking” antibody or an “antagonist” antibody is one which inhibits or reduces biological activity of the antigen it binds. Certain blocking antibodies or antagonist antibodies substantially or completely inhibit the biological activity of the antigen.

An “agonist antibody”, as used herein, is an antibody which mimics at least one of the functional activities of a polypeptide of interest.

A “disorder” is any condition that would benefit from treatment with an ADC of the present disclosure. This includes chronic and acute disorders or diseases including those pathological conditions which predispose the mammal to the disorder in question. Non-limiting examples of disorders to be treated herein include cancer.

The terms “cell proliferative disorder” and “proliferative disorder” refer to disorders that are associated with some degree of abnormal cell proliferation. In one embodiment, the cell proliferative disorder is cancer.

The term “tumor” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

The terms “cancer” and “cancerous” refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, multiple myeloma and leukemia. More particular examples of such cancers include lung cancer, breast cancer, head-and-neck cancer, esophagus cancer, stomach cancer, bladder cancer, pancreatic cancer, colorectal cancer, cervix cancer, endometrial cancer, ovarian cancer, laryngeal cancer, prostate cancer, thyroid cancer and oral cancer.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing or decreasing inflammation and/or tissue/organ damage, decreasing the rate of disease progression, amelioration or palliation of the disease state, and remission or improved prognosis.

An “individual” or a “subject” is a vertebrate. In certain embodiments, the vertebrate is a mammal. Mammals include, but are not limited to, farm animals (such as cows), sport animals, pets (such as cats, dogs, and horses), primates, mice and rats. In certain embodiments, the vertebrate is a human.

A “combination therapy” refers to a combination of an amount of an ADC and an amount of other biological or chemical drugs that when administered together (either as co-administration and/or co-formulation), either sequentially or simultaneously, on the same or different days during a treatment cycle, have a synergistic effect that is therapeutically effective and more than therapeutically additive.

The term “cytotoxic agent” as used herein refers to a substance that inhibits or prevents the function of cells and/or causes destruction of cells. Cytotoxic agents include radionuclides such as radioactive isotopes (e.g., ²¹¹ At, ¹³¹I, ¹²⁵I, ⁹⁰Y, ¹⁸⁶Re, ¹⁸⁸Re, ¹⁵³Sm, ²¹²Bi, ³²P, ⁶° C., and radioactive isotopes of lutetium-177, strontium-89 and samarium (153Sm)), chemotherapeutic agents, and toxins such as small molecule toxins or enzymatically active toxins of bacterial, fungal, plant or animal origin, including synthetic analogs and derivatives thereof.

The term “photodynamic therapy (PDT)”, sometimes called photochemotherapy, is a form of phototherapy involving light and a photosensitizing chemical substance, used in conjunction with molecular oxygen to elicit cell death (phototoxicity). It is used clinically to treat a wide range of medical conditions, including wet age-related macular degeneration, psoriasis, atherosclerosis and has shown some efficacy in anti-viral treatments, including herpes. It also treats malignant cancers including head and neck, lung, bladder, skin and prostate cancer (Wang, S S et al. Cancer Journal. 8 (2): 154-63. 2002). The “photodynamic therapeutic agent” is selected from Photofrin, Laserphyrin, Aminolevulinic acid (ALA), Silicon Phthalocyanine Pc 4, m-tetrahydroxyphenylchlorin (mTHPC), chlorin e6 (Ce6), Allumera, Levulan, Foscan, Metvix, Hexvix, Photochlor, Photosens, Photrex, Lumacan, Visonac, Amphinex, Verteporfin, Purlytin, ATMPn, Zinc phthalocyanine (ZnPc), Protoporphyrin IX (PpIX), Pyropheophorbidea (PPa) or Pheophorbide a (PhA).

A “chemotherapeutic agent” is a chemical compound useful in the treatment of cancer. Examples of chemotherapeutic agents include Monomethyl auristatin E (MMAE), Monomethyl auristatin F (MMAF), mertansine (also called DM1), anthracycline, pyrrolobenzodiazepine, α-amanitin, tubulysin, benzodiazepine, erlotinib (TARCEVA®), Genentech/OSI Pharm.), bortezomib (VELCADE®, Millenium Pharm.), fulvestrant (FASLODEX®, Astrazeneca), sunitinib (SUTENT®, SU11248, Pfizer), letrozole (FEMARA®), Novartis), imatinib mesylate (GLEEVEC®, Novartis), PTK787/ZK 222584 (Novartis), oxaliplatin (ELOXATIN®, Sanofi), leucovorin, rapamycin (Sirolimus, RAPAMUNE®, Wyeth), lapatinib (TYKERBR, GSK572016, GlaxoSmithKline), lonafarnib (SARASAR®, SCH 66336), sorafenib (NEXAVAR®, BAY43-9006, Bayer Labs.), and gefitinib (IRESSA®, Astrazeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone), camptothecin (including the synthetic analogue topotecan), bryostatin, callystatin, CC-1065 (including its adozelesin, carzelesin and bizelesin synthetic analogues), cryptophycins (particularly cryptophycin 1 and cryptophycin 8), dolastatin, duocarmycin (including the synthetic analogues, KW-2189 and CB1-TM1), eleutherobin, pancratistatin, sarcodictyin, spongistatin, nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, especially calicheamicin gamma1I and calicheamicin omegaI1), dynemicin, including dynemicin A; aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone, aldophosphamide glycoside, aminolevulinic acid, eniluracil, amsacrine, bestrabucil, bisantrene, edatraxate, defofamine, demecolcine, diaziquone, elformithine, elliptinium acetate, epothilone, etoglucid, gallium nitrate, hydroxyurea, lentinan, lonidainine, maytansinoids such as maytansine and ansamitocins; mitoguazone, mitoxantrone, mopidanmol, nitraerine, pentostatin, phenamet, pirarubicin, losoxantrone, podophyllinic acid, 2-ethylhydrazide, procarbazine, razoxane, rhizoxin, sizofiran, spirogermanium, tenuazonic acid, triaziquone, 2,2′,2″-trichlorotriethylamine, trichothecenes (especially T-2 toxin, verracurin A, roridin A and anguidine), urethan, vindesine, dacarbazine, mannomustine, mitobronitol, mitolactol, pipobroman, gacytosine, arabinoside (“Ara-C”), cyclophosphamide, thiotepa, taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil, GEMZAR® gemcitabine, 6-thioguanine, mercaptopurine, methotrexate, platinum analogs such as cisplatin and carboplatin; vinblastine, platinum, etoposide (VP-16), ifosfamide, mitoxantrone, vincristine, NAVELBINE® vinorelbine, novantrone, teniposide, edatrexate, daunomycin, aminopterin, xeloda, ibandronate, CPT-11, topoisomerase inhibitor RFS 2000, difluoromethylornithine (DMFO), retinoids such as retinoic acid, capecitabine (XELODA®, Roche), and pharmaceutically acceptable salts, acids or derivatives of any of the above.

The phrase “chemotherapeutic agent” may act as one or more of the following: (i) anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON, toremifene; (ii) aromatase inhibitors that inhibit the enzyme aromatase, which regulates estrogen production in the adrenal glands, such as, for example, 4 (5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; (iii) anti-androgens such as flutamide, nilutamide, bicalutamide, leuprolide, and goserelin; as well as troxacitabine (a 1,3-dioxolane nucleoside cytosine analog); (iv) aromatase inhibitors; (v) protein kinase inhibitors; (vi) lipid kinase inhibitors; (vii) antisense oligonucleotides, particularly those which inhibit expression of genes in signaling pathways implicated in adherent cell proliferation, such as, for example, PKC-alpha, Ralf and H-Ras; (viii) ribozymes such as a VEGF expression inhibitor (e.g., ANGIOZYME® ribozyme) and a HER2 expression inhibitor; (ix) vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; (x) anti-angiogenic agents such as bevacizumab (AVASTIN®, Genentech); and (xi) pharmaceutically acceptable salts, acids or derivatives of any of the above.

The “protein kinase inhibitors” include tyrosine kinase inhibitors which inhibit to some extent tyrosine kinase activity of a tyrosine kinase such as an ErbB receptor. Examples of tyrosine kinase inhibitors include EGFR-targeted drugs such as: (i) antibodies which bind to EGFR, including MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBITUX®, Imclone) and reshaped human 225 (H225) (WO 96/40210, Imclone Systems Inc.); antibodies that bind type II mutant EGFR (U.S. Pat. No. 5,212,290); humanized and chimeric antibodies that bind EGFR (U.S. Pat. No. 5,891,996); and human antibodies that bind EGFR, such as ABX-EGF (WO 98/50433); (ii) anti-EGFR antibody conjugated with a cytotoxic agent (EP 659439A2); and small molecules that bind to EGFR including ZD1839 or Gefitinib (IRESSA™; Astra Zeneca), Erlotinib HCl (CP-358774, TARCEVA™; Genentech/OSI) and AG1478, AG1571 (SU 5271; Sugen), quinazolines such as PD 153035,4-(3-chloroanilino) quinazoline, pyridopyrimidines, pyrimidopyrimidines, pyrrolopyrimidines, such as CGP 59326, CGP 60261 and CGP 62706, and pyrazolopyrimidines, 4-(phenylamino)-7H-pyrrolo[2,3-d]pyrimidines, curcumin (diferuloylmethane, 4,5-bis(4-fluoroanilino) phthalimide), tyrphostines containing nitrothiophene moieties; PD-0183805 (Warner-Lambert); antisense molecules (e.g., those that bind to ErbB-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-ErbB inhibitors such as CI-1033 (Pfizer); Affinitac (ISIS 3521; Isis/Lilly); Imatinib mesylate (Gleevac; Novartis); PKI 166 (Novartis); GW2016 (Glaxo SmithKline); CI-1033 (Pfizer); EKB-569 (Wyeth); Semaxanib (Sugen); ZD6474 (AstraZeneca); PTK-787 (Novartis/Schering AG); INC-1C11 (Imclone); or as described in: U.S. Pat. No. 5,804,396; WO 99/09016 (American Cyanamid); WO 98/43960 (American Cyanamid); WO 97/38983 (Warner Lambert); WO 99/06378 (Warner Lambert); WO 99/06396 (Warner Lambert); WO 96/30347 (Pfizer, Inc); WO 96/33978 (Zeneca); WO 96/3397 (Zeneca); and WO 96/33980 (Zeneca).

An “anti-angiogenic agent” refers to a compound which blocks, or interferes with to some degree, the development of blood vessels. The anti-angiogenic factor may, for instance, be a small molecule or antibody that binds to a growth factor or growth factor receptor involved in promoting angiogenesis. An exemplary anti-angiogenic agent is an antibody that binds to Vascular Endothelial Growth Factor (VEGF) such as bevacizumab (AVASTIN®, Genentech).

The term “cytokine” refers to a protein released by one cell population which acts on another cell as intercellular mediators. Cytokines may include lymphokines, monokines, and traditional polypeptide hormones. Examples of cytokines include growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); granulocyte-CSF (G-CSF); interleukins (ILs) such as IL-1, IL-1α, IL-2, IL-3, IL-4, IL-5, IL-6, IL-7, IL-8, IL-9, IL-10, IL-11, IL-12; a tumor necrosis factor such as TNF-α or TNF-β; and other polypeptide factors including LIF and kit ligand (KL). Cytokines may have natural or artificial sources (e.g., from recombinant cell culture) and include biologically active equivalents.

The term “prodrug” as used herein refers to a precursor or derivative form of a pharmaceutically active substance that is less cytotoxic to tumor cells compared to its parent form and is capable of being enzymatically activated or converted into the more active parent form. Examples of prodrugs include, but are not limited to, phosphate-containing prodrugs, thiophosphate-containing prodrugs, sulfate-containing prodrugs, peptide-containing prodrugs, D-amino acid-modified pro drugs, glycosylated prodrugs, β-lactam-containing prodrugs, optionally substituted phenoxyacetamide-containing prodrugs or optionally substituted phenylacetamide-containing prodrugs, 5-fluorocytosine, or other 5-fluorouridine prodrugs which can be converted into the more active cytotoxic drug. Examples of cytotoxic drugs that can be derivatized into a prodrug form for use in ADCs include, but are not limited to, those chemotherapeutic agents described above.

The term “liposome” refers to a small vesicle composed of various types of lipids, phospholipids and/or surfactant which is useful for delivery of a drug (such as the anti-HER2 antibodies disclosed herein and, optionally, a chemotherapeutic agent) to a mammal. The components of the liposome are commonly arranged in a bilayer formation, similar to the lipid arrangement of biological membranes.

The phrase “pharmaceutically acceptable salt,” as used herein, refers to pharmaceutically acceptable organic or inorganic salts of a cytotoxic agent or an ADC. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

The term “pharmaceutically acceptable solvate” refers to an association of one or more solvent molecules with a cytotoxic agent or an ADC. Examples of solvents that form pharmaceutically acceptable solvates include, but are not limited to, water, isopropanol, ethanol, methanol, DMSO, ethyl acetate, acetic acid, and ethanolamine.

### Antibody Conjugates

The present disclosure provides an antibody conjugate prepared by conjugating a linker-payload as described herein with an antibody or an antigen-binding fragment thereof. Depending on the nature of the linker-payload, which may be a drug-linker compound (DL) that includes a drug unit as the payload or a probe-linker that includes a probe unit, the antibody conjugate may be an antibody-drug conjugate or an antibody-probe conjugate.

In certain embodiments, the antibody-drug conjugates (ADC) is represented by Formula (I):

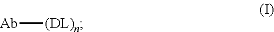

wherein Ab is an antibody or an antigen-binding fragment thereof capable of binding to one or more of tumor-associated antigens or cell-surface receptors;
DL is a linker-payload as described herein where the payload is a drug unit, and a bioorthogonal group in DL forms a covalent linkage with the antibody or the antigen-binding fragment thereof; and
n is a drug-to-antibody ratio (DAR) ranging from 1 to 20.

In previous studies, the linker-payload may have the structure of Formula (II):

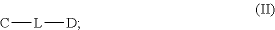

wherein C is a bioorthogonal group for conjugation to an antibody or an antigen-binding fragment thereof;
L is a linker unit including a protease-cleavable peptide moiety, a glycosidase-cleavable sugar moiety, a pH sensitive moiety, or a hydrolysable moiety; and
D is a drug unit.

In contrast, the present disclosure provides the linker-payload having the structure of Formula (III):

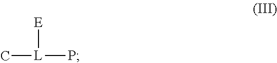

wherein E is a hydrophilic moiety including: polyethylene glycol (PEG), polysarcosine (pSar), poly lactic-co-glycolic acid (PLGA), poly(glycerols) (PGs), poly(oxazolines) (POX), poly(hydroxypropyl methacrylate) (PHPMA), poly(2-hydroxyethyl methacrylate) (PHEMA), poly(N-(2-hydroxypropyl) methacrylamide) (HPMA), poly(vinylpyrrolidone) (PVP), poly(N,N-dimethyl acrylamide) (PDMA), poly(N-acryloylmorpholine) (PACM), saccharides, or any combination thereof;
C is a bioorthogonal group for conjugation to an antibody or an antigen-binding fragment thereof;
L is a linker unit including a protease-cleavable peptide moiety, a glycosidase-cleavable sugar moiety, a pH sensitive moiety, or a hydrolysable moiety; and
P is a payload selected from a drug unit or a probe unit.

In certain embodiments, the hydrophilic moiety has a molecular weight ranging from 60 Da to 13 kDa, from 100 Da to 10 kDa, from 200 Da to 9 kDa, from 250 Da to 8 kDa, from 300 Da to 7 kDa, from 350 Da to 6 kDa, from 400 Da to 5 kDa, from 450 Da to 4 kDa, from 500 Da to 3 kDa, from 600 Da to 3 kDa, from 700 Da to 3 kDa, from 800 Da to 3 kDa, from 900 Da to 3 kDa, or from 1 kDa to 3 kDa, or from 500 Da to 2.5 kDa. In certain embodiments, the hydrophilic moiety includes PEG with or without other chemical moieties at one or two ends of the PEG, and the PEG may have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 ethylene glycol units, or the ethylene glycol units may range between any two of the numbers listed above.

The term “bioorthogonal group (C)” refers to a non-native chemical group that can be used to conjugate the linker-payload with an antibody or an antigen-binding fragment thereof under the conditions of living systems without affecting the activity of the antibody or antigen-binding fragment thereof. In certain embodiments, the bioorthogonal group is selected from a dibenzocyclooctyne (DBCO) group, a bicyclononyne (BCN) group, a alkyne group, a maleimide group, a α,β-unsaturated carbonyl group, a sulfonyl pyrimidine group, a 4-dibenzocyclooctynol (DIBO) group, a aza-dibenzocyclooctynes (DIBAC) group, a tetrazine group, a tetrazole group, a norbornene group, a cyclooctyne group, a methylcyclopropene group, an aminooxy group, a hydrazine group, an isocyanide group, an isocyanopropanoate group, a phosphine-containing thioester group, a phosphine phenolic ester group, or an alpha-halo carbonyl group. In certain embodiments, the bioorthogonal group is an alkyne group, which can react with an azide introduced at a specific site on an antibody through metal-free azide-alkyne cycloaddition, resulting in antibody conjugation via a triazole linkage. In other embodiments, the bioorthogonal group is a cyclooctyne, DBCO, or BCN group, which can react with an azide on an antibody (e.g., an azide on an N-glycan at an asparagine residue in an antibody heavy chain) without a catalyst via strain-promoted azide-alkyne cycloaddition to achieve antibody conjugation via a triazoline linkage. In other embodiments, the bioorthogonal group is a tetrazine group, which can react with a trans-cyclooctene (TCO) on an antibody to form a dihydropyridazine linkage, resulting in antibody conjugation.

The term “linker unit (L)” refers to an element connecting the bioorthogonal group with the payload and including a cleavable moiety. In certain embodiments, the linker unit is a protease-cleavable peptide moiety, a glycosidase-cleavable sugar moiety, a pH sensitive moiety or a hydrolysable moiety, each of which being directly linked with the bioorthogonal group and/or directly linked with the payload. In certain embodiments, the linker unit includes any one or more of the listed cleavable moieties and an intervening spacer (usually with a molecular weight of between 15 Da and 1800 Da) for connecting with the bioorthogonal group or the payload. The spacer may be a di-functional or tri-functional chemical moiety that is capable of covalently linking together the cleavable moiety of the linker unit and any one or two of the bioorthogonal group, the payload, and the hydrophilic moiety. Examples of the spacer include but not limited to amino acids and aminobenzyl alcohols.

The protease-cleavable peptide moiety may be a peptide composed of two or more natural or non-natural amino acids that can be cleaved by a peptidase. Preferably, the protease-cleavable peptide moiety may be a dipeptide selected from valine-alanine (VA), valine-cysteine (VC), phenylalanine-glycine (FG), phenylalanine-lysine (FK), alanine-alanine (AA), glycine-valine (GV), or glycine-cysteine (GC); alternatively, a peptide may be composed of said dipeptide and one to eight additional amino acids, for example, a tetrapeptide where two additional glycine or alanine residues are attached to the N-terminal of said dipeptide.

The glycosidase-cleavable sugar moiety may a sugar residue linked via an oxygen glycosidic bond to a self-immolative group, for example, a glucuronic acid linked via an oxygen glycosidic bond to a p-aminobenzyl alcohol linker, which can be cleaved by β-glucuronidase.

The pH sensitive moiety may be an acid-liable moiety that can be hydrolyzed at acidic pH and optionally an intracellularly cleavable moiety that is cleavable in the low pH environment of endosomal and lysosomal vesicles. The acidic pH condition is beneficial to release payload from the ADC. Examples of the pH sensitive moiety may be one or more amino acids spacers between the drug and the linker.

The hydrolysable moiety may be a chemical moiety cleaved by hydrolase. Preferably, the hydrolase is an esterase.

The term “payload,” as used herein, refers to a molecule to be carried and delivered by an antibody or an antigen-binding fragment thereof. The term “drug unit” refers to a drug molecule (e.g., a cytotoxic agent), an inhibitor of an enzyme, a ligand of a receptor, or a pharmaceutically acceptable salt, a pharmaceutically acceptable solvate, or a prodrug thereof. The term “probe unit” refers to a probe molecule that assists the visualization of target cells or tissues or a body part of a subject. In certain embodiments, the payload is selected from a chemotherapeutic agent, a toxin, a cytokine, a growth inhibitory agent, a protein degrader, a peptide, a radionuclide, a hormone, an anti-viral agent, an anti-bacterial agent, an immunoregulatory agent, a fluorophore, a dye, or a contrast agent.

In certain embodiments, the toxin is selected from pyrrolobenzodiazepine compounds or derivatives thereof (e.g., PBD), auristatin compounds or derivatives thereof (e.g., MMAE, MMAF), maytansinoid compounds or derivatives thereof (e.g., maytansine, DM1, DM4, DM21), duocarmycin or derivatives thereof, nicotinamide phosphoribosyltransferase (NAMPT) inhibitors or derivatives thereof, tubulysin compounds or derivatives thereof, enediyne compounds or derivatives thereof (e.g., calicheamicin), anthracycline compounds or derivatives thereof (e.g., doxorubicin), pyrrole-based kinesin spindle protein (KSP) inhibitors or derivatives thereof, cryptophycin compounds or derivatives thereof (e.g., cryptophycin 52), drug efflux pump inhibitors or derivatives thereof, sandramycin or derivatives thereof, amanitin compounds or derivatives thereof, or camptothecin compounds or derivatives thereof (e.g., SN-38, belotecan, exatecan, deruxtecan).

In certain embodiments, the linker unit (L) of Formula (III) has the structure of L^{P}-Q^{CL}-Q^{SP}, and the linker-payload is represented by Formula (IV):

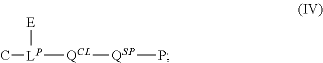

wherein:
(a) C is a bioorthogonal group for conjugation to an antibody or an antigen-binding fragment thereof;
(b) E is a hydrophilic moiety as defined in Formula (III);
(c) Q^{SP} is a spacer unit including an aromatic group or amino methylene;
(d) Q^{CL} is a cleavable unit including a protease-cleavable peptide moiety, a glycosidase-cleavable sugar moiety, a pH sensitive moiety, or a hydrolysable moiety;
(e) L^{P} is a connector unit that covalently connects Q^{CL}, C and E, wherein the connector unit includes one or more amino acids; and
(f) P is a payload selected from a drug unit or a probe unit.

In certain embodiments, E of Formula (IV) includes PEG and may have the formula of:

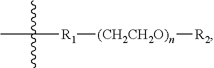

wherein the wavy line indicates the site of covalent attachment to L^{P},
R₁ is —C(O)—, —O—, —S—, —NH—, —C(O)O—, alkyl-C(O)—NH—, alkyl-NH—C(O)—, alkyl-CO₂—, alkyl-S—, or

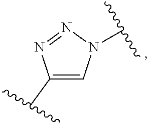 [SMILES: *c1cn(*)nn1]

R₂ is selected from H, SO₃H, PO₃H₂, a sugar derivative, C₁-C₁₀ (hetero)alkyl group, C₃-C₁₀ (hetero)cycloalkyl group, C₂-C₁₀ alkyl-NH₂, C₁-C₁₀ alkyl-COOH, C₂-C₁₀ alkyl-NH(C₁-C₃ alkyl), C₂-C₁₀ alkyl-N(C₁-C₃ alkyl)₂, or sarcosines, and the subscript n is an integer ranging from 2 to 72.

In certain embodiments, the linker unit (L) of Formula (III) has the structure of L^{B}-Q^{CL}-B^{P}, and the linker-payload is represented by Formula (V):

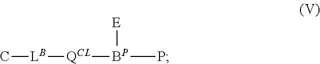

wherein:
(a) C is a bioorthogonal group for conjugation to an antibody or an antigen-binding fragment thereof;
(b) E is a hydrophilic moiety as defined in Formula (III);
(c) B^{P} is a branch unit that covalently connects Q^{CL}, E and P, wherein the branch unit includes a functional group defined as

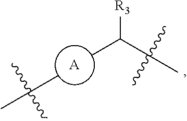

wherein
A is an aromatic group;
R₃ is linked to E and is selected from —C(O)—, —C(O)O—, —C(O)NH—, alkyl-O—, alkyl-NH—, alkyl-C(O)—, alkyl-C(O)—O—, alkyl-C(O)—NH—, alkyl-NH—C(O)—, alkyl-SO₂—, alkyl-S—, alkyl-O—P(O)—O₂—, alkyl-O—C(O)—NH—, or triazole; and the bond extending directly from one benzene carbon of the functional group is covalently linked to Q^{CL};
(d) Q^{CL} is a cleavable unit including a protease-cleavable peptide moiety, a glycosidase-cleavable sugar moiety, a pH sensitive moiety, or a hydrolysable moiety;
(e) L^{B} is a bridge unit that covalently connects C and Q^{CL}, and
(f) P is a payload selected from a drug unit or a probe unit.

In certain embodiments, E of Formula (V) includes PEG and may have the formula of:

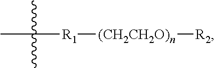

wherein the wavy line indicates the site of covalent attachment to B^{P},
R₁ is —C(O)—, —O—, —S—, —NH—, —C(O)O—, alkyl-C(O)—NH—, alkyl-NH—C(O)—, alkyl-CO₂—, alkyl-S—, or

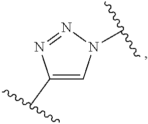 [SMILES: *c1cn(*)nn1]

R₂ is selected from H, SO₃H, PO₃H₂, a sugar derivative, C₁-C₁₀ (hetero)alkyl group, C₃-C₁₀ (hetero)cycloalkyl group, C₂-C₁₀ alkyl-NH₂, C₁-C₁₀ alkyl-COOH, C₂-C₁₀ alkyl-NH(C₁-C₃ alkyl), C₂-C₁₀ alkyl-N(C₁-C₃ alkyl)₂, or sarcosines, and the subscript n is an integer ranging from 2 to 72.

In certain embodiments, the linker-payload of Formula (V) includes (i) the drug unit selected from camptothecin compounds or derivatives thereof (such as exatecan) and (ii) the hydrophilic moiety including PEG. Said linker-payload is represented by Formula (VI):

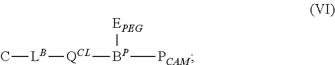

wherein E_{PEG} refers to the hydrophilic moiety including PEG, and P_{CAM} refers to the drug unit selected from camptothecin compounds or derivatives thereof. The E_{PEG} may have the formula of:

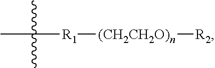

wherein the wavy line indicates the site of covalent attachment to B^{P},
R₁ is —C(O)—, —O—, —S—, —NH—, —C(O)O—, alkyl-C(O)—NH—, alkyl-NH—C(O)—, alkyl-CO₂—, alkyl-S—, or

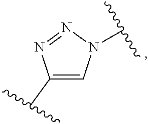 [SMILES: *c1cn(*)nn1]

R₂ is selected from H, SO₃H, PO₃H₂, a sugar derivative, C₁-C₁₀ (hetero)alkyl group, C₃-C₁₀ (hetero)cycloalkyl group, C₂-C₁₀ alkyl-NH₂, C₁-C₁₀ alkyl-COOH, C₂-C₁₀ alkyl-NH(C₁-C₃ alkyl), C₂-C₁₀ alkyl-N(C₁-C₃ alkyl)₂, or sarcosines, and
the subscript n is an integer ranging from 2 to 72.

In certain embodiments, the linker-payload has the structure of the following formula:

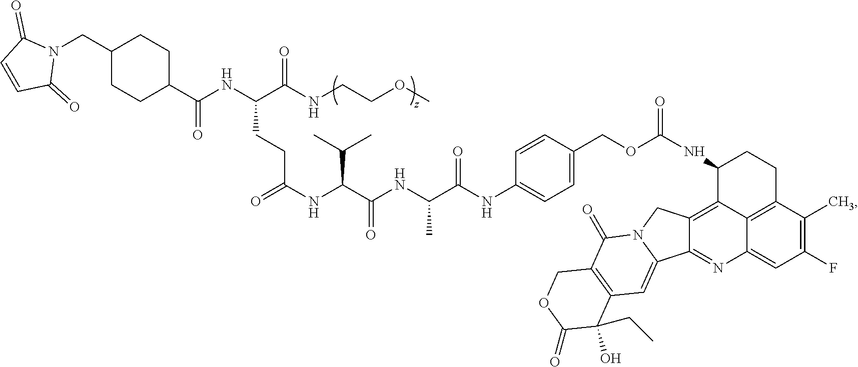

wherein z is an integer ranging from 6 to 48. In one embodiment, z is 24, and the linker-payload has the structure of Formula (VII) (referred to as MCCA-PEG₂₄-VA-PAB-Exatecan or DL-1):

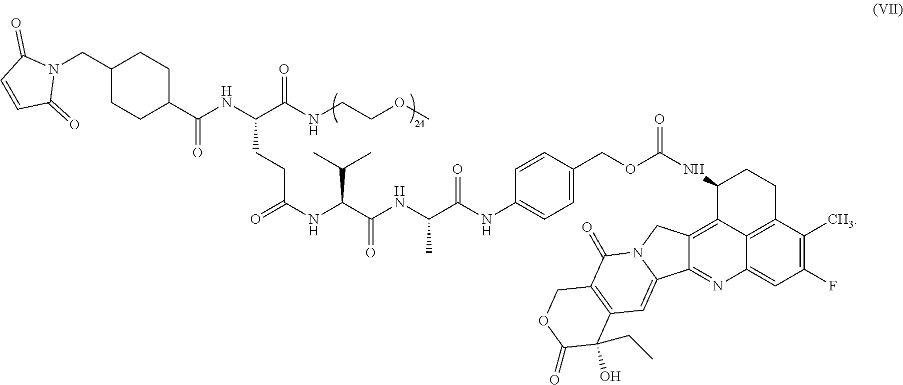

The compound of Formula (VII) is an example of the compound of Formula (IV), where Q^{CL} includes a protease-cleavable dipeptide of valine and alanine. The dipeptide is connected to PAB, an example of Q^{SP} that further connects exatecan. The dipeptide is also connected to L^{P} that includes a glutamic acid residue via the sidechain of the said residue. The L^{P} further connects an MCCA group (an example of the maleimide group) at N-terminal end of the glutamic acid residue and also connects PEG at C-terminal end of the said residue via an amide linkage.

In certain embodiments, the linker-payload has the structure of the following formula:

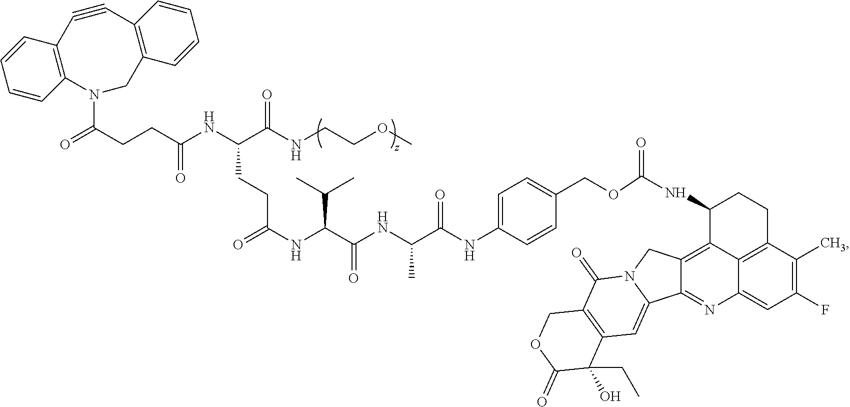

wherein z is an integer ranging from 6 to 48. In one embodiment, z is 24, and the linker-payload has the structure of Formula (VIII) (referred to as DBCO-PEG₂₄-VA-PAB-Exatecan or DL-2):

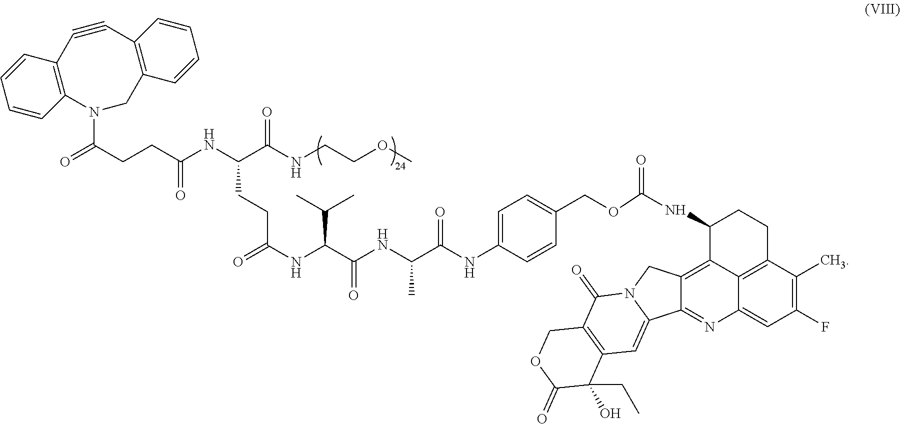

In certain embodiments, the linker-payload has the structure of the following formula:

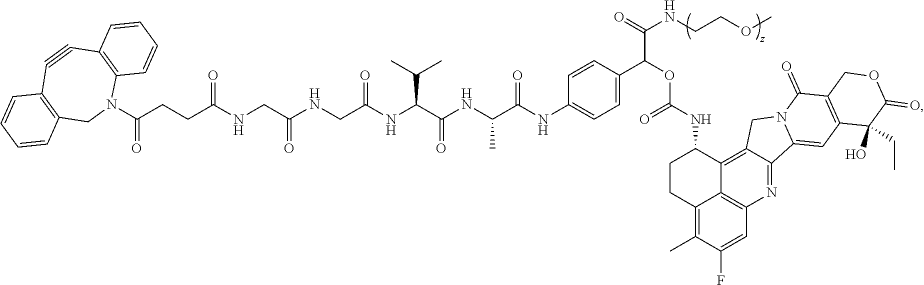

wherein z is an integer ranging from 6 to 48.

In certain embodiments, the linker-payload has the structure of the following formula:

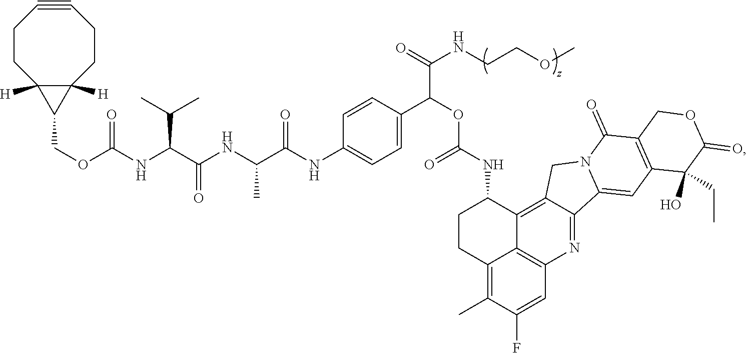

wherein z is an integer ranging from 6 to 48.

In one embodiment, the linker-payload has the structure of Formula (IX) (referred to as BCN-GGVA-Hydra-PAB-PEG₂₄-Exatecan or DL-8):

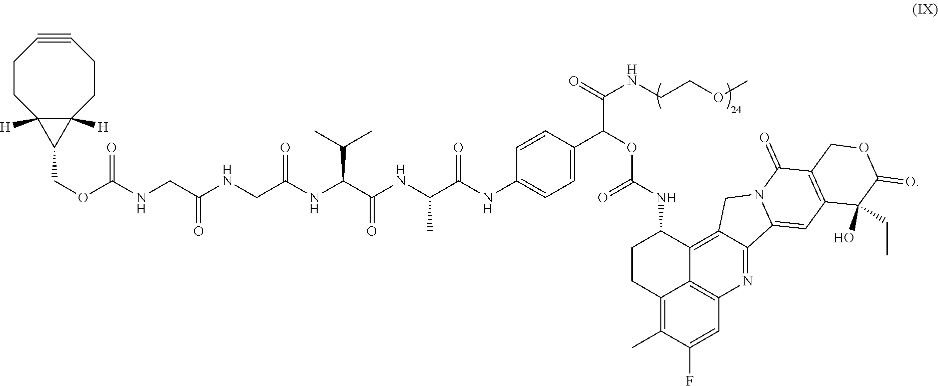

In certain embodiments, the linker unit (L) of Formula (III) has the structure of (L₁)ₙ₁-Q^{CL}-(L₂)ₙ₂, and the linker-payload is represented by Formula (X):

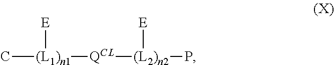

wherein:
(a) E is a hydrophilic moiety as defined in Formula (III);
(b) C is a bioorthogonal group for conjugation to an antibody or an antigen-binding fragment thereof;
(c) L₁ and L₂ are the same or different connector unit, wherein the connector unit includes one or more amino acid or a functional group defined as

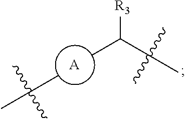

wherein
A is an aromatic group;
R₃ is linked to E and is selected from —C(O)—, —C(O)O—, —C(O)NH—, alkyl-O—, alkyl-NH—, alkyl-C(O)—, alkyl-C(O)—O—, alkyl-C(O)—NH—, alkyl-NH—C(O)—, alkyl-SO₂—, alkyl-S—, alkyl-O—P(O)—O₂—, alkyl-O—C(O)—NH—, or triazole; and the bond extending directly from one benzene carbon of the functional group is covalently linked to Q^{CL},
n1 and n2 are independently 0, 1 or 2, but n1 and n2 are not 0 at the same time;
(d) Q^{CL} is a cleavable unit including a protease-cleavable peptide moiety, a glycosidase-cleavable sugar moiety, a pH sensitive moiety, or a hydrolysable moiety; and
(e) P is a payload selected from a drug unit or a probe unit.

In certain embodiments, the antibody or antigen-binding fragment thereof of the ADC is monospecific or multispecific. In certain embodiments, the antibody or antigen-binding fragment thereof is capable of specifically binding to CLDN18.2, B7-H₃, MUC-1, PD-1, PD-L1, CTLA-4, VEGF, BCMA, PSMA, CGRP, Tfr1, TNF alpha, tissue factor, folate receptor alpha, c-MET, HER3, EGFR, TROP2, HER2, or Nectin-4. In one preferred embodiment, the antibody or antigen-binding fragment thereof is bispecific to HER2 and TROP2. In another embodiment, the antibody or antigen-binding fragment thereof is bispecific to c-MET and HER3. In another embodiment, the antibody or antigen-binding fragment thereof is bispecific to EGFR and HER3. In another embodiment, the antibody or antigen-binding fragment thereof is bispecific to EGFR and c-Met.

### Preparation of Antibody Conjugates

The present disclosure further provides a method for preparing an antibody-drug conjugate (ADC), including reacting an antibody with the linker-payload disclosed herein to obtain the ADC, wherein the payload is a drug unit. The ADC prepared accordingly has enhanced tumor suppression efficacy and/or improved circulation stability when administered to a subject in need.

In some embodiments, the ADC preparation method further includes the step of adding a co-solvent into a reaction mixture including the linker-payload and the antibody. In some embodiments, the linker-payload includes a hydrophilic moiety that includes PEG, and the co-solvent is propylene glycol. In some embodiments, the hydrophilic moiety of the linker-payload includes PEG having 1 to 48 ethylene glycol units, 6 to 48 ethylene glycol units, 12 to 48 ethylene glycol units, or 24 to 48 ethylene glycol units. Preferably, the hydrophilic moiety of the linker-payload includes PEG having 12 to 48 ethylene glycol units. In some embodiments, the propylene glycol has a volume ratio ranging from 0-50% to the total volume of the reaction mixture.

The present disclosure further provides a method for preparing a glycan-engineered antibody conjugate, including reacting a glycan-engineered antibody with the linker-payload disclosed herein to obtain the glycan-engineered antibody conjugate, wherein the glycan-engineered antibody includes a fucosylated or non-fucosylated N-acetylglucosamine (GlcNAc) at an asparagine residue coupled to a glycan.

In some embodiments, the glycan-engineered antibody is obtained by contacting an antibody with a glycosynthase and a glycan oxazoline to couple the glycan with the fucosylated or non-fucosylated GlcNAc. In some embodiments, the glycosynthase includes EndoSd-D232M and EndoSz-D234M.

### Therapeutic Applications

The present disclosure further provides a method for killing or inhibiting the proliferation of tumor cells or cancer cells, including contacting the cells with an effective amount of the ADC disclosed herein.

The present disclosure also provides a method for treating a disease, including administering to a subject in need thereof an effective amount of the ADC disclosed herein or a pharmaceutical composition that includes one or more of the ADCs described herein. By varying the characteristics of the ADC (such as the type of the antibody or the payload), the disease to be treated may be cancer, infection (including viral and bacterial infections), nervous system disorders, inflammatory disorders, or autoimmune disorders.

In some embodiments, the subject (e.g., a human patient) in need of the treatment is diagnosed with, suspected of having, or at risk for cancer. Examples of the cancer include, but are not limited to, multiple myeloma, acute myeloid leukemia (AML), sarcoma, skin cancer, leukemia, lymphoma, brain cancer, glioblastoma, lung cancer, breast cancer, head-and-neck cancer, nasopharyngeal cancer, esophagus cancer, stomach cancer, liver cancer, bile duct cancer, gallbladder cancer, bladder cancer, pancreatic cancer, intestinal cancer, colorectal cancer, kidney cancer, cervix cancer, endometrial cancer, ovarian cancer, testicular cancer, buccal cancer, oropharyngeal cancer, laryngeal cancer, prostate cancer, thyroid cancer, and oral cancer.

In some embodiments, the treatment results in reduction of tumor size, elimination of malignant cells, prevention of metastasis, prevention of relapse, reduction or killing of disseminated cancer, prolongation of survival and/or prolongation of time to tumor cancer progression.

In some embodiments, the method for treatment further includes administering an additional therapy to said subject prior to, during or subsequent to said administering of the ADCs. In some embodiments, the additional therapy is treatment with a chemotherapeutic agent. In some embodiments, the additional therapy is radiation therapy, photodynamic therapy, chemotherapy, immunotherapy, targeted therapy, or hormone therapy.

The methods for treating cancers described herein are particularly advantageous in treating and preventing early stage tumors, thereby preventing progression to the more advanced stages resulting in a reduction in the morbidity and mortality associated with advanced cancer. The methods are also advantageous in preventing the recurrence of a tumor or the regrowth of a tumor, for example, a dormant tumor that persists after removal of the primary tumor, or in reducing or preventing the occurrence of a tumor.

The subject to be treated by the treatment methods described herein can be a mammal, more preferably a human. Mammals include, but are not limited to, farm animals, sport animals, pets, primates, horses, dogs, cats, mice, or rats. A human subject who needs the treatment may be a human patient having, at risk for, or suspected of having cancer, which include, but are not limited to, lung cancer, breast cancer, head-and-neck cancer, esophagus cancer, stomach cancer, bladder cancer, pancreatic cancer, colorectal cancer, cervix cancer, endometrial cancer, ovarian cancer, laryngeal cancer, prostate cancer, thyroid cancer, or oral cancer. A subject having cancer can be identified by medical examination.

The phrase “an effective amount” refers to the amount of each active agent or a pharmaceutical composition required to achieve the desired therapeutic result (“therapeutically effective amount”) or the desired prophylactic result (“prophylactically effective amount”), either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

As used herein, the term “treating” refers to the application or administration of a composition including one or more active agents to a subject, who has a disease such as cancer, a symptom of cancer, or a predisposition toward cancer, with the purpose to cure, heal, alleviate, relieve, alter, remedy, ameliorate, improve, or affect cancer, the symptom of cancer, or the predisposition toward cancer, or to delay the development or progression of cancer. “Development” or “progression” of cancer means initial manifestations and/or ensuing progression of cancer. Development of cancer can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein, “onset” or “occurrence” of cancer includes initial onset and/or recurrence.

### Applications in Imaging

The present disclosure further provides a method of selecting a subject for a cancer therapy by imaging, including:
(a) administering to the subject an effective amount of the antibody conjugate disclosed herein where the payload is the probe unit, wherein the probe unit is an imaging agent selected from a fluorophore, a dye, a contrast agent, or a radionuclide;
(b) detecting visually or instrumentally a reporting signal of the imaging agent in the subject; and
(c) identifying the subject as suitable for the cancer therapy when the reporting signal is detected.

In certain embodiments, the subject is diagnosed with, suspected of having, or at risk for cancer.

In certain embodiments, the method further comprises detecting metastasis of a cancer.

### Administration of ADC, Pharmaceutical Compositions, and Pharmaceutical Formulations

The present disclosure also provides a pharmaceutical composition including the ADC described herein and a pharmaceutically acceptable carrier. Conventional methods, known to those of ordinary skill in the art of medicine, can be used to administer an ADC or a pharmaceutical composition including an ADC to the subject, depending upon the type of disease to be treated or the site of the disease. A pharmaceutical composition can be administered via various routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques.

Pharmaceutical formulations of therapeutic ADCs are typically prepared for parenteral administration with a pharmaceutically acceptable parenteral vehicle and in a unit dosage injectable form. An ADC having the desired degree of purity is optionally mixed with pharmaceutically acceptable diluents, carriers, excipients or stabilizers, in the form of a lyophilized formulation or an aqueous solution (Remington's Pharmaceutical Sciences (1980)₁₆th edition, Osol, A. Ed.).

Acceptable parenteral vehicles, diluents, carriers, excipients, and stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). For example, lyophilized anti-HER2 antibody formulations are described in WO 97/04801, expressly incorporated herein by reference. An exemplary formulation of an ADC such as trastuzumab-SMCC-DM1 contains about 100 mg/mL of trehalose (2-(hydroxymethyl)-6-[3,4,5-trihydroxy-6-(hydroxymethyl)tetrahydropyran-2-yl]oxy-tetrahydropyran-3,4,5-triol; C₁₂H₂₂O₁₁; CAS Number 99-20-7) and about 0.1% TWEEN™ 20 (polysorbate 20; dodecanoic acid 2-[2-[3,4-bis(2-hydroxyethoxy)tetrahydrofuran-2-yl]-2-(2-hydroxyethoxy) ethoxy]ethyl ester; C₂₆H₅₀O₁₀; CAS Number 9005-64-5) at approximately pH 6.

The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions.

Sustained-release preparations may be prepared. Suitable examples of sustained-release preparations include semi permeable matrices of solid hydrophobic polymers containing the ADC, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile, which is readily accomplished by filtration through sterile filtration membranes.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

The pharmaceutical compositions of ADC may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables. Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble ADCs can be administered by the drip method, whereby a pharmaceutical formulation containing the ADCs and a physiologically acceptable excipients is infused. Aqueous suspensions contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

The amount of active ingredient that may be combined with the carrier material to produce a single dosage form will vary depending upon the host treated and the particular mode of administration. For example, an aqueous solution intended for intravenous infusion may contain from about 3 to 500 μg of the active ingredient per milliliter of solution in order that infusion of a suitable volume at a rate of about 30 mL/hour can occur. Subcutaneous (bolus) administration may be effected with about 1.5 ml or less of total volume and a concentration of about 100 mg ADC per ml. For ADC that require frequent and chronic administration, the subcutaneous route may be employed, such as by pre-filled syringe or autoinjector device technology.

As a general proposition, the initial effective amount of ADC administered per dose will be in the range of about 0.01-100 mg/kg, namely about 0.1 to 20 mg/kg of patient body weight per day, with the typical initial range of compound used being 0.3 to 15 mg/kg/day. The dose may be escalated to the maximally tolerated dose (MTD). The dosing schedule may be about every 3 weeks, but according to diagnosed condition or response, the schedule may be more or less frequent. The dose may be further adjusted during the course of treatment to be at or below MTD which can be safely administered for multiple cycles, such as about 4 or more.

Although oral administration of protein therapeutics are generally disfavored due to poor bioavailability due to limited absorption, hydrolysis or denaturation in the gut, formulations of ADC suitable for oral administration may be prepared as discrete units such as capsules, cachets or tablets each containing a predetermined amount of the ADC.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Exemplary unit dosage formulations contain a daily dose or unit daily sub-dose, or an appropriate fraction thereof, of the active ingredient.

The present disclosure further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

For repeated administrations over several days or longer, depending on the condition, the treatment is sustained until a desired suppression of disease symptoms occurs. The progress of this therapy is easily monitored by conventional techniques and assays.

### Combination Therapy

The ADC described herein may be combined in a pharmaceutical combination formulation, or dosing regimen as combination therapy, with a second therapeutic agent having anti-cancer properties. The second therapeutic agent of the pharmaceutical combination formulation or dosing regimen preferably has complementary and/or synergistic activities to the ADC of the combination such that they do not adversely affect each other.

The second therapeutic agent may be a chemotherapeutic agent, a cytotoxic agent, a cytokine, a growth inhibitory agent, an anti-hormonal agent, an aromatase inhibitor, a protein kinase inhibitor, a lipid kinase inhibitor, an anti-androgen, an antisense oligonucleotide, a ribozyme, a gene therapy vaccine, an anti-angiogenic agent, a cardioprotectant, or an immunotherapeutic agent. Such molecules are suitably present in combination in amounts that are effective for the purpose intended. A pharmaceutical composition containing an ADC may also have a therapeutically effective amount of a chemotherapeutic agent such as a tubulin-forming inhibitor, a topoisomerase inhibitor, or a DNA binder.

### Articles of Manufacture

In another embodiment, an article of manufacture, or “kit”, containing ADC and materials useful for the treatment of the disorders described above is provided. The article of manufacture comprises a container and a label or package insert on or associated with the container. Suitable containers include, for example, bottles, vials, syringes, or blister pack. The containers may be formed from a variety of materials such as glass or plastic. The container holds an ADC composition which is effective for treating the condition and may have a sterile access port (for example the container may be an intravenous solution bag or a vial having a stopper pierceable by a hypodermic injection needle). At least one active agent in the composition is an ADC. The label or package insert indicates that the composition is used for treating the condition of choice, such as cancer, infection, nervous system, inflammatory, or autoimmune disorders.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1. Preparation of NSCT Derivates

### 1-1. Preparation of NSCT-2 (Oxazoline-NSCT-N3)

NSCT-1 (235 mg, 0.097 mmol; purchased from Glytech, Inc. Catalog No. GT-25261; HPLC purity >90%) and triethylamine (605 μL, 0.44 mmol) were dissolved in water (10 mL) and cooled to 0° C. 2-chloro-1,3-dimethyl-1H-benzimidazol-3-ium chloride aqueous solution (1 M, 1.44 mL) was added slowly and the resulting mixture was stirred at 0 to 5° C. for 4 hours. NaOH solution was added (0.01 M, 1 mL) and the resulting mixture was concentrated under reduced pressure. After most of the triethylamine was evaporated, the residual mixture was purified by Sephadex® g-15 column. Using 0.01 M NaOH as eluent to stabilize NSCT-2. Fractions with desired product were combined and freeze-dried to afford NSCT-2 (170 mg) as a white solid. 1H NMR (D₂O): δ 6.10 (d, J=7.26 Hz, 1H, H1 of oxazoline), 5.24 (s, 1H, H1 of GluNAc), 4.97 (s, 1H, H1 of GluNAc), 4.76 (s, 1H, H1 of Man), 4.64-4.60 (m, 2H, H1 of two Gal), 4.46 (s, 1H, H1 of Neu5Ac), 4.45 (s, 1H, H1 of Neu5Ac), 4.40 (s, 1H, H3 of β-form Man), 4.18 (d, J=21 Hz, 4H, H2 of two Man and two GluNAc), 3.99-3.48 (m), 2.71 (dd, J1=12.9 Hz, J2=4.2 Hz, 2H, H3 eq of two Neu5Ac), 2.15-2.02 (m, 15H), 1.58 (dd, J1=J2=12.2 Hz, 2H, H3ax of two Neu5Ac).

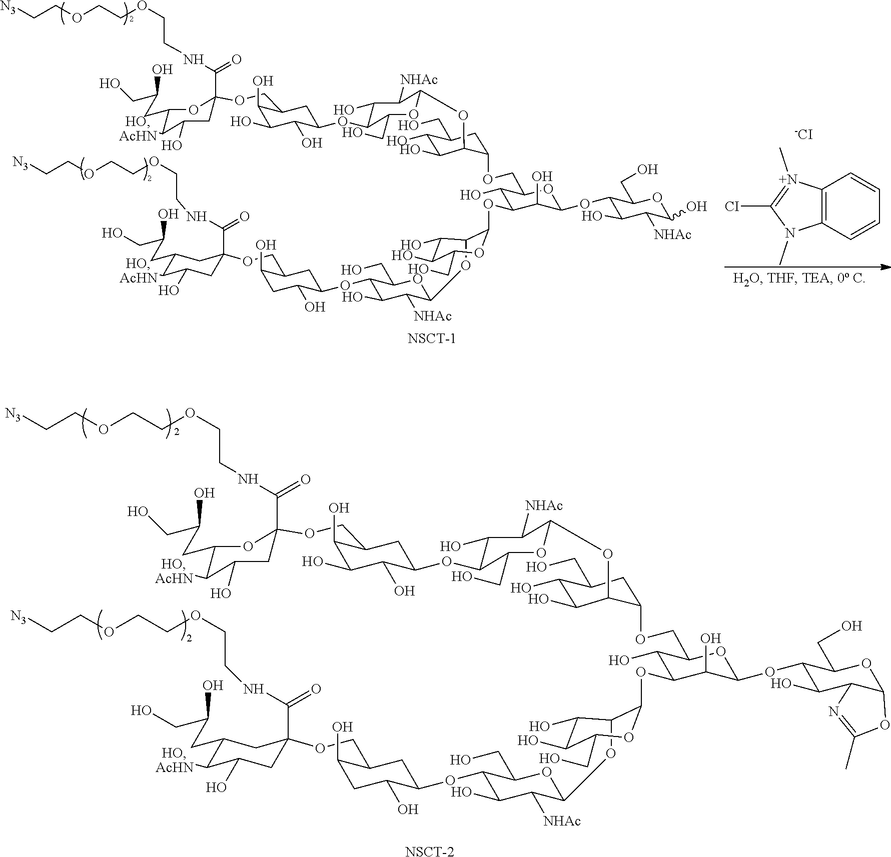

### 1-2. Preparation of NSCT-5

NSCT (50.0 mg, 0.0247 mmol; purchased from Glytech Inc.) dissolved in DMF (2.0 mL) with DBCO—NH₂ (41.0 mg, 0.148 mmol), PyBOP (257.0 mg, 0.494 mmol) and DIPEA (63.8 mg, 0.494 mmol) was added. This mixture was stirred under room temperature for 20 hours. After the reaction was completed, the reaction mixture was extracted with DCM/H₂O. The H₂O layer was collected and lyophilized to afford a crude product. The crude product was purified by reverse phase chromatography (eluent: ACN/Water). The pure fractions were combined and lyophilized to obtain NSCT-5 (41.5 mg, 66.2% yield). MS calculate for [C₁₁₂H₁₅₃N₉O₅₇+2Na⁺]/2:1290.9573, found: 1290.9553. ¹H NMR (D₂O): δ 7.69 (t, 2H), 7.52-7.59 (m, 12H), 7.48 (t, 2H), 7.43 (t, 2H), 7.37 (t, 2H), 5.12 (t, 4H), 4.59 (t, 4H), 4.43 (dd, 2H), 4.28 (s, 1H), 4.21 (s, 1H), 4.13 (s, 1H), 4.05-3.47 (m), 3.38-3.46 (m, 2H), 3.35-3.23 (m, 2H), 3.21-3.12 (m, 2H) 2.58-2.42 (m, 3H), 2.09-2.00 (m), 1.76 (td, 1H).

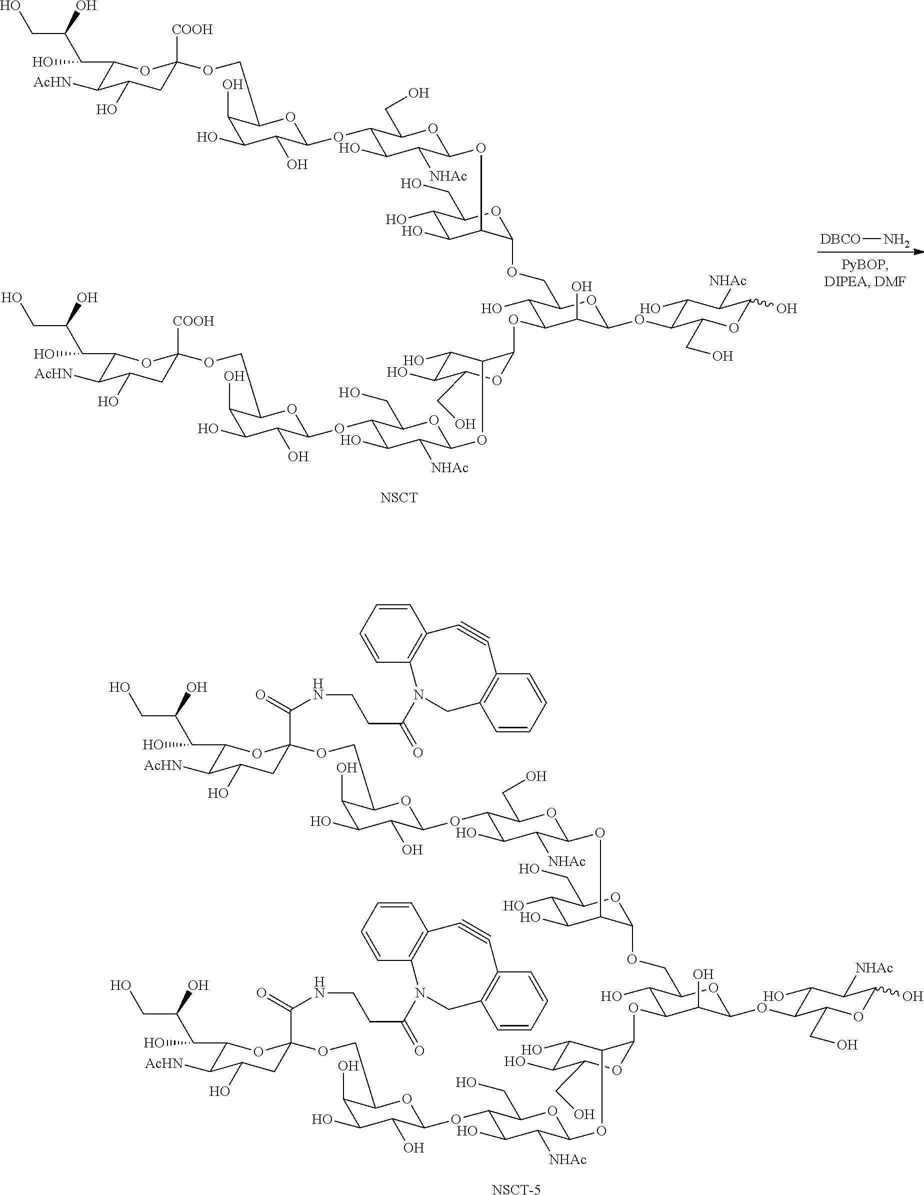

### 1-3. Preparation of NSCT-6 (Oxazoline-NSCT-DBCO)

A method similar to that described in NSCT-2 preparation was applied to obtain the oxazoline product NSCT-6 from NSCT-5.

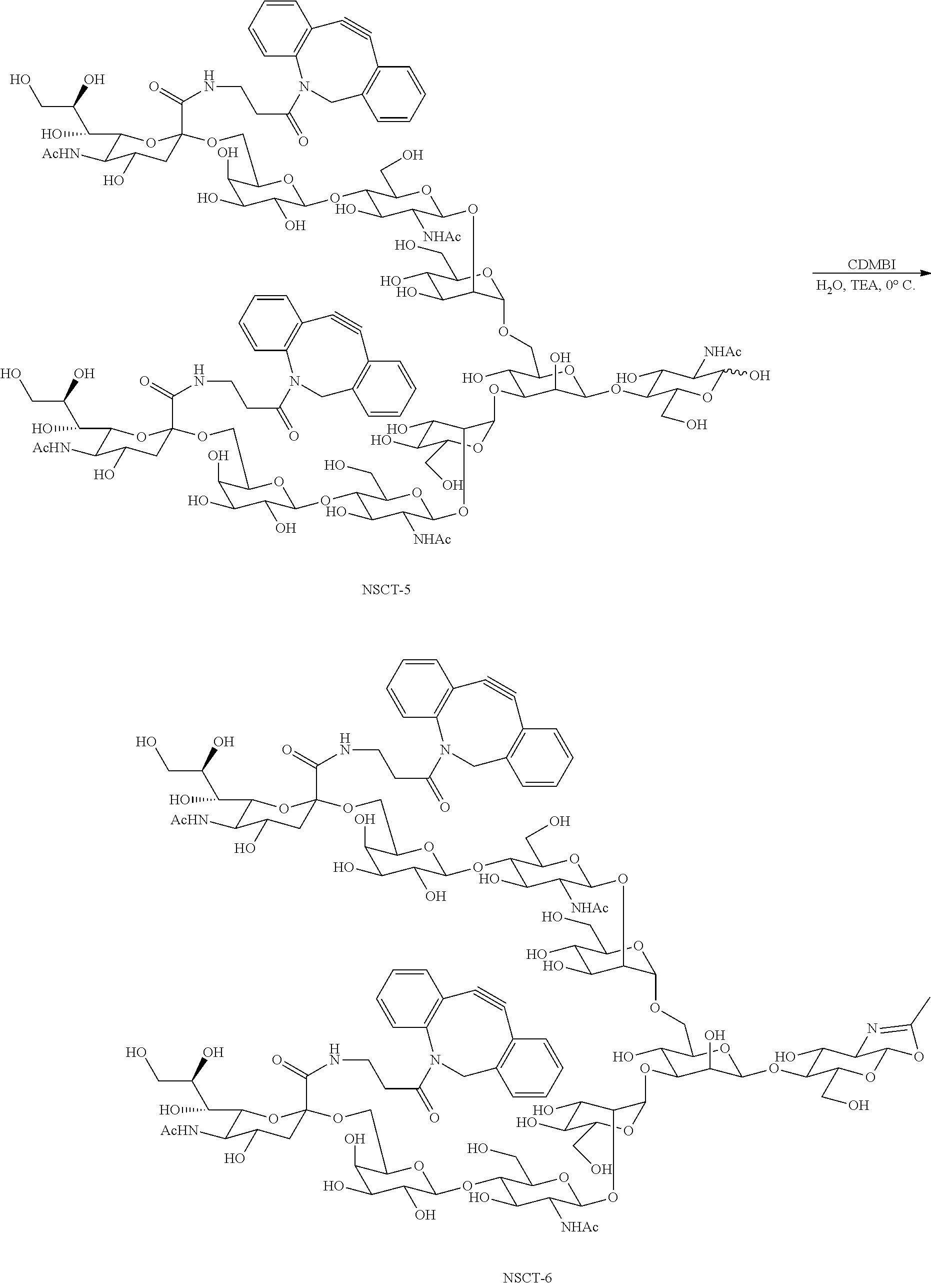

### Example 2. Deglycosylation, Transglycosylation and Purification of Monoclonal Antibodies

2-1. Deglycosylation of mAbs by EndoSz-D234M to Generate mAb-GlcNAc (Fuc)

Two monoclonal antibodies were used to illustrate the process to obtain glycan-engineered antibodies. R4702 was an anti-TROP2 monoclonal antibody and TX05 was an anti-HER2 monoclonal antibody purchased from Tanvex BioPharma Inc. The monoclonal antibodies were deglycosylated with EndoSz-D234M in 50 mM Tris pH 7.2 at 37° C. for 24-49 hours. Only the antibodies with high mannose N-glycan modification were further supplied with EndoH and incubated at 25° C. overnight to remove glycans completely and produced mAb-GlcNAc (Fuc). The complete cleavage of Fc N-glycans were analyzed by SDS-PAGE and CE-SDS.

The EndoSz-D234M was applied to other mAbs for further deglycosylation and transglycosylation investigation. For the deglycosylation studies, the mAbs were incubated with EndoSz-D234M at a weight ratio of 1:30 (EndoSz-D234M: mAbs). All the mAb-GlcNAc (Fuc) from different mAbs reached to >90% by EndoSz-D234M cleavage. For the transglycosylation investigation with NSCT-2, 20 or 38 equivalents of NSCT-2 was added to mAb-GlcNAc (Fuc) for incubation with EndoSz-D234M at 37° C. for 1.5 to 2 hours. Table 1 showed that the yields of mAb-(NSCT-di-N3)₂ were 95.61% for R4702-(NSCT-di-N3)₂ and 96.7% for TX05-(NSCT-di-N3)₂, analyzed by CE-SDS. These data indicate that EndoSz-D234M can be applied to various mAbs.

TABLE 1
Deglycosylation and transglycosylation of R4702 and TX05 mAbs

| | mAb | | | |
|---|---|---|---|---|
| | R4702 | | TX05 | |
| | mAb- | mAb- | mAb- | mAb- |
| Form | GlcNAc(Fuc) | (NSCT-di-N3)₂ | GlcNAc(Fuc) | (NSCT-di-N3)₂ |
| HC- | 96.42% | 2.54% | 94.5% | 1.3% |
| GlcNAC | | | | |
| HC | 3.58% | 1.85% | 5.5% | 2.0% |
| HC-N3 | — | 95.61% | — | 96.7% |

### 2-2. Transglycosylation of mAb-GlcNAc (Fuc) with Oxazoline-NSCT-N3 (NSCT-2) to Generate mAb-(NSCT-di-N3)₂

In general, mAb-GlcNAc (Fuc) were incubated with 20-38 equivalents of NSCT-2 at 37° C. for 1.5-2 hours to generate mAb-(NSCT-di-N3)₂. The transglycosylation efficiency was monitored by SDS-PAGE and CE-SDS.

2-3. Purification of mAb-(NSCT-di-N3)₂

Sodium chloride was added into the transglycosylation mixture to reach a final concentration of 3M and then applied to PBS and 3M NaCl pre-equilibrated HiTrap Phenyl HP (Cytiva). The non-bound contaminations were washed by 5CV of equilibration buffer (PBS and 3M NaCl). mAb-(NSCT-di-N3)₂ was eluted with a 30-100% elution buffer (Sodium phosphate 20 mM and 20% IPA pH 7.2) in 20CV linear gradient. The eluted fractions were applied to a prepacked column, HiTrap Protein A HP (Cytiva). The impurities were washed by two steps pH gradient, 100 mM Sodium citrate pH 6.0 and pH 5.5, with 5CV in each step. 50 mM Sodium citrate pH 3.5 was employed to elute bound antibody. The eluted fractions were immediately neutralized with 1 M Tris-HCl pH 9.0 to natural pH and change buffer to 20 mM Sodium Acetate pH 5.0 with Amicon centrifugation membrane (30 kDa cutoff, Millipore). The purified mAb-(NSCT-di-N3)₂ were stored at −80° C.

### Example 3. Synthesis of Drug-Linker Compound

### 3-1. Preparation of MCCA-PEG₂₄-VA-PAB-Exatecan (Also Termed DL-1/N-PM-0017)

### Step 1:

To a suspension of exatecan mesylate in DMF, N-PM-0015 and DIPEA were added at room temperature. The suspension became a clear brown solution within 5 minutes. This mixture was stirred at room temperature for 20 hours. After the reaction was completed, the reaction mixture was added to a stirring TBME to get precipitate. After stirring for 30 minutes, the solids were collected by filtration and followed with high vacuum drying to obtain crude N-PM-0016. This crude product was used in the next step without further purification.

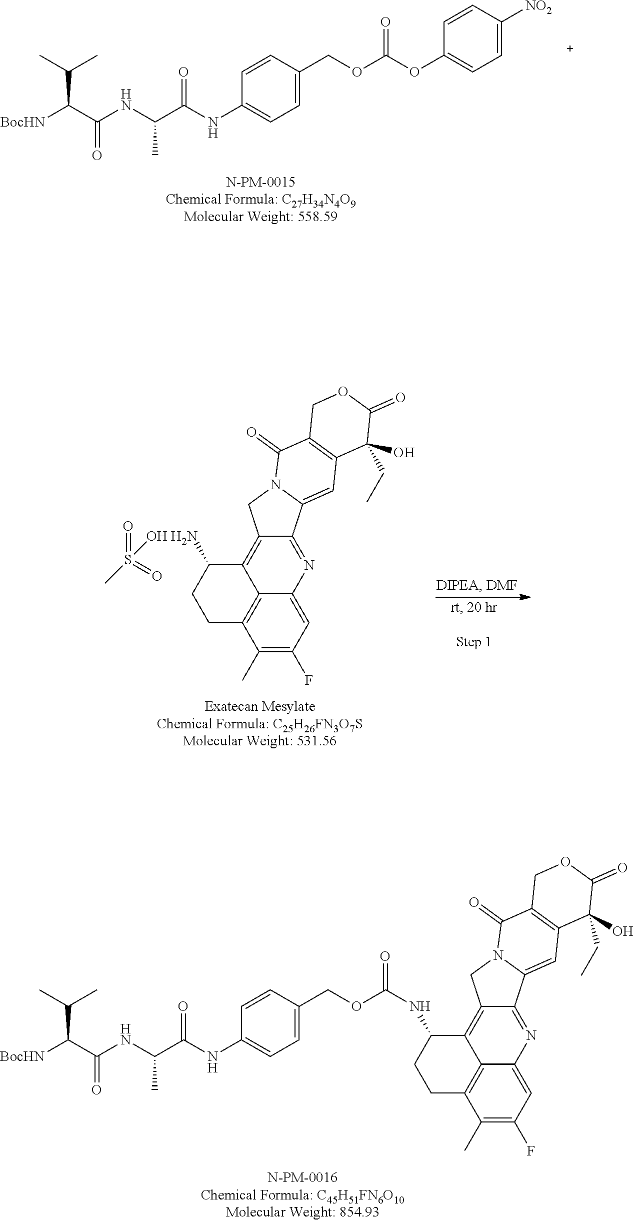

### Step 2:

A stirring suspension of N-PM-0016 in DCM was cooled to −20° C. A−10° C. pre-cooled TFA liquid was added to N-PM-0016 solution over 60 minutes. This mixture was stirred at −20° C. for 10 hours. After the reaction was completed, the reaction mixture was added to a stirring TBME to get precipitate. After stirring for 30 minutes, the solids were collected by filtration and followed with high vacuum drying to obtain crude N-PM-0018. This crude product was used in the next step without further purification.

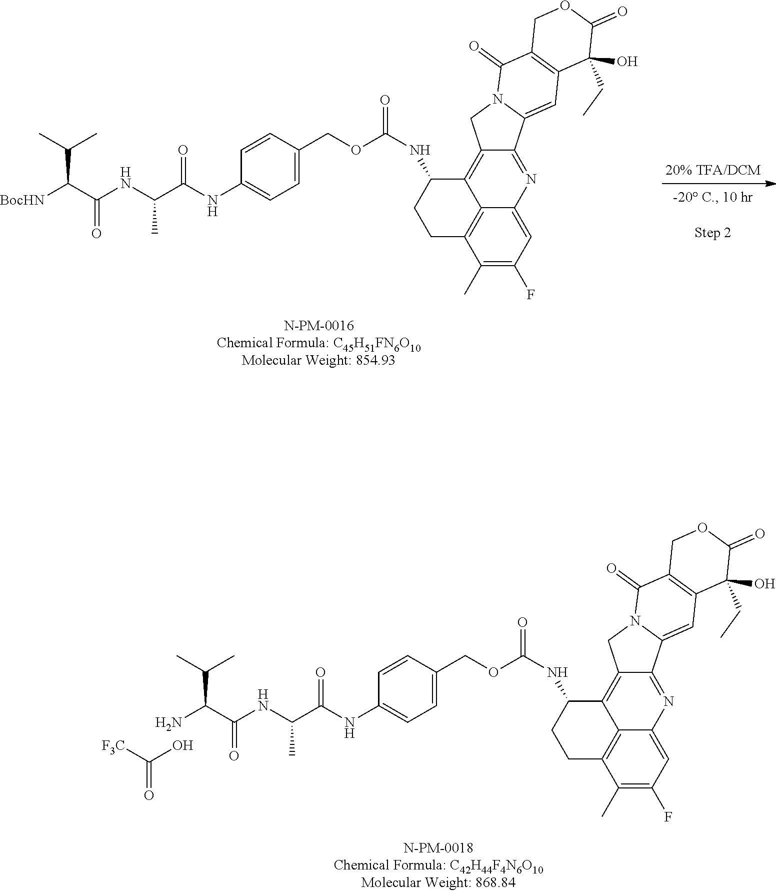

### Step 3:

A solution of N-DT-0013 in DMF, HATU and NMM was added. This mixture was stirred at room temperature for 2 hours. A solution of N-PM-0018 and NMM in DMF was added to the N-DT-0013 solution at room temperature over 30 minutes. This mixture was stirred at room temperature for further 2 hours. After the reaction was completed, the reaction mixture was added to a stirring TBME to get precipitate. After stirring for 30 minutes, the solids were collected by filtration and purified by reverse phase chromatography (eluent: ACN/Water). The pure fractions were combined and extracted with 10% MeOH/DCM to obtain N-PM-0017.

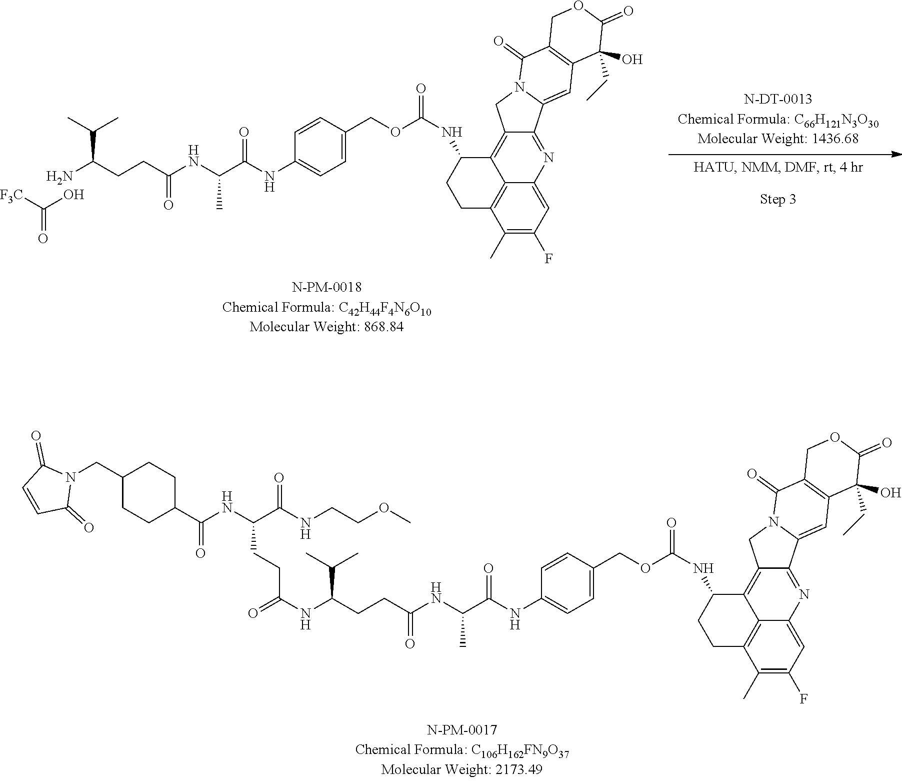

### 3-2. Preparation of DBCO-PEG₂₄-VA-PAB-Exatecan (Also Termed DL-2)

### Step 1:

N-(9-Fmoc)-L-glutamic acid g-tert-butyl ester monohydrate (152.2 mg, 0.35 mmol), m-PEG24-amine (380.9 mg, 0.35 mmol) and HATU (159.7 mg, 0.42 mmol) were dissolved in DMF/CH₂Cl₂=1/1 (3.5 mL) under room temperature. NMM (115.8 μL, 1.05 mmol) was added. After the addition, the resulting mixture was stirred at room temperature for 18 hours. The reaction solution was concentrated in vacuo at 30-35° C., and the residue was purified by a flash silica gel column (CH₂Cl₂/MeOH=15/1 to 12/1) to obtain 502.5 mg of Glu-1 with 96.0% yield.

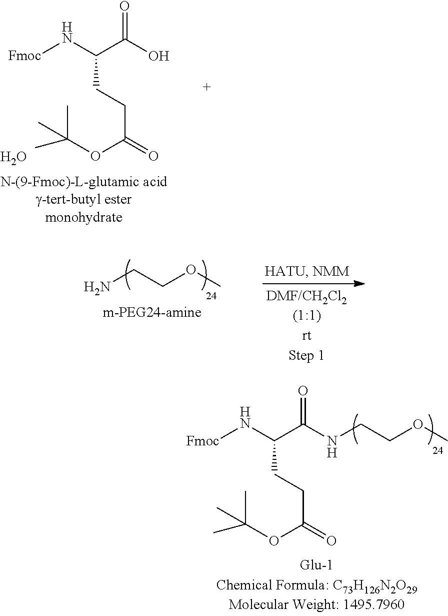

### Step 2:

To a solution of Glu-1 (413.8 mg, 0.27 mmol) in CH₂Cl₂/MeOH=1/1 (13.8 ml), Et₂NH (1.38 ml) was added. The mixture was stirred at room temperature for 24 hours. After the reaction was completed, the reaction mixture was concentrated in vacuo at 30-35° C. and then azeotroped with toluene (5 mL×3) to remove excess Et₂NH. The resulting material was dried in high vacuum to obtain crude Glu-2. The crude product was used in the next step without further purification.

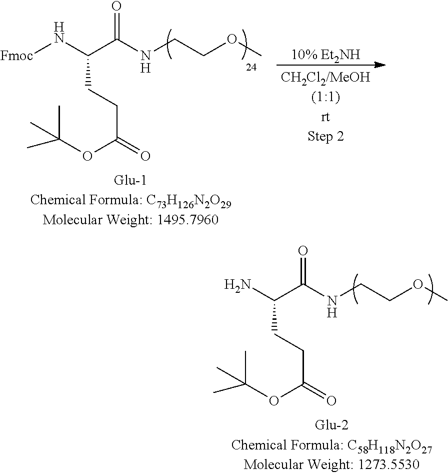

### Step 3:

To a solution of crude Glu-2 (352.3 mg, 0.27 mmol) and DBCO-acid (101.4 mg, 0.33 mmol) in DMF/CH₂Cl₂=1/1 (5.5 ml), HATU (157.8 mg, 0.45 mmol) and NMM (91.6 μL, 0.83 mmol) were added separately. The reaction mixture was stirred at room temperature for 16 hours. After the reaction was completed, the reaction solution was concentrated in vacuo at 30-35° C., and the residue was purified with a flash silica gel column (CH₂Cl₂/MeOH=15/1) to obtain 367.9 mg of DBCO-1 with 85.2% yield from Glu-1.

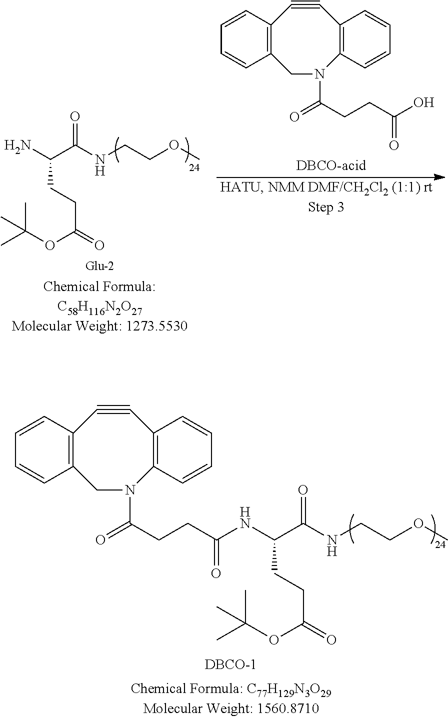

### Step 4:

A solution of DBCO-1 (350.0 mg, 0.22 mmol) in CH₂Cl₂ (6.9 mL) was cooled to 0° C. TFA (1.8 mL) was added dropwise. The reaction mixture was stirred at 0-4° C. for 4-6 hours. After the reaction was completed, the reaction mixture was concentrated in vacuo at 30-35° C., and the residue was purified with a flash silica gel column (CH₂Cl₂/MeOH=12/1) to obtain 175.4 mg of DBCO-2 with 52.0% yield.

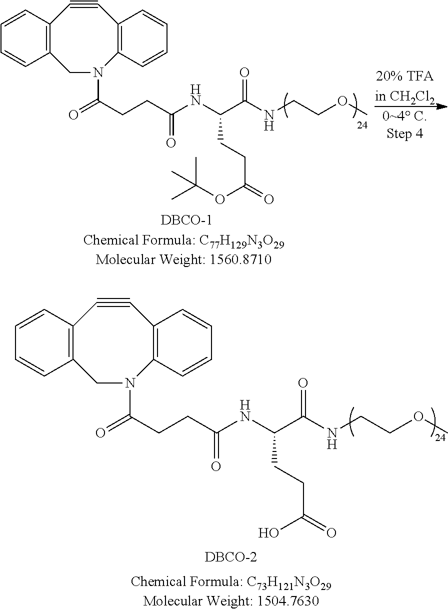

### Step 5:

N-PM-0018 (26.5 mg, 0.031 mmol), DBCO-2 (45.9 mg, 0.031 mmol) and HATU (13.9 mg, 0.037 mmol) were dissolved in DMF (0.61 mL). NMM (10.1 μL, 0.092 mmol) was added. The reaction mixture was stirred at room temperature for 20 hours. After the reaction was completed, the resulting mixture was purified with preparative HPLC to obtain 48.9 mg of DL-2 with 71.5% yield. ¹H NMR (600 MHz, d-MeOH) δ 7.70-7.50 (m, 6H), 7.46-7.36 (m, 5H), 7.33-7.10 (m, 3H), 5.56 (d, 1H, J=16.0 Hz), 5.36 (dd, 1H, J=16.0, 6.2 Hz), 5.33-5.29 (m, 1H), 5.27 (d, 1H, J=20.2 Hz), 5.21-5.13 (m, 2H), 5.13-5.05 (m, 2H), 4.47-4.41 (m, 1H), 4.23-4.12 (m, 2H), 3.73-3.52 (m, 102H), 3.50-3.46 (m, 1H), 3.43-3.38 (m, 1H), 3.35 (s, 3H), 3.27-3.17 (m, 1H), 3.15-3.07 (m, 1H), 2.81-2.70 (m, 1H), 2.37 (s, 3H), 2.36-2.24 (m, 4H), 2.45-2.40 (m, 1H), 2.24-2.00 (m, 5H), 2.00-1.78 (m, 4H), 1.43 (d, 1H, J=7.1 Hz), 1.03-0.94 (m, 9H); HRMS (ESI) m/z found [(M+2H)/2]⁺, 1121.0605 C₁₁₃H₁₆₄FN₉O₃₆²⁺, required 1121.0553.

TABLE 2
HPLC condition (Column:
YMC-Actus Trait C18 250 × 20 mm, 5 μm, 12 nm)

| Gradient: | | | |
|---|---|---|---|
| Time | Flow (mL/minute) | H₂O | ACN |
| 0 min | 10 | 90% | 10% |
| 1 min | 20 | 70% | 30% |
| 5 min | 20 | 55% | 45% |
| 20 min | 20 | 45% | 55% |
| 21 min | 20 | 10% | 90% |
| 30 min | 20 | 10% | 90% |

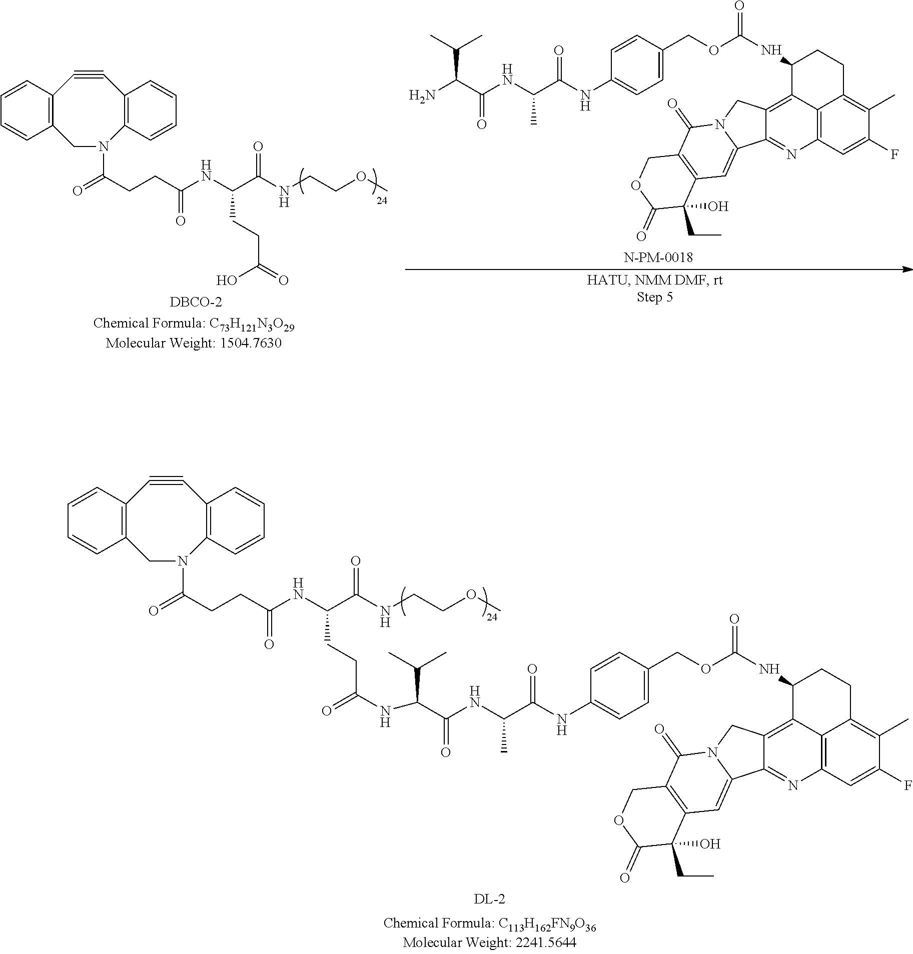

### 3-3. Preparation of MCCA-Linear PEG₁₂-VA-PAB-Exatecan (Also Termed DL-3)

Mal-2 (14.0 mg, 0.0162 mmol) in DMF (0.6 mL) was cooled to 0-4° C. N-PM-0018 (16.9 mg, 0.0194 mmol), NMM (4.1 mg, 0.0405 mmol) and HOAt (5.5 mg, 0.0405 mmol) were added sequentially under 0-4° C. This mixture was stirred at 0-4° C. for 16 hours. After the reaction was completed, the reaction mixture was purified by reverse phase chromatography (eluent: ACN/Water). The pure fractions were combined and extracted with 10% MeOH/DCM to obtain DL-3 (13.6 mg, 55.8% yield). MS calculate for [C₇₄H₁₀₁FN₈O₂₄+2H+]/2:753.3529, found: 753.3534.

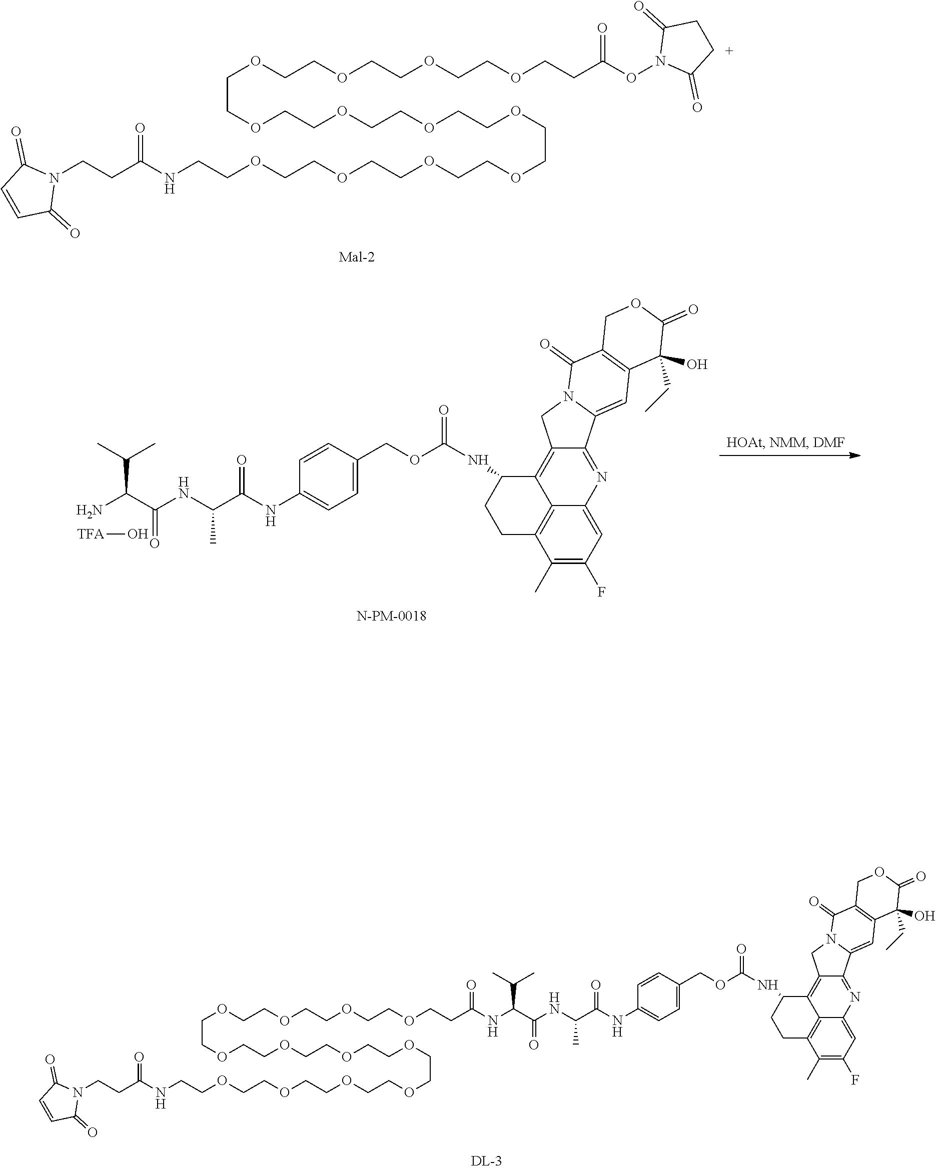

### 3-4. Preparation of DBCO-VA-Hydra-PAB-PEG₂₄-Exatecan (Also Termed DL-5)

### Step 1:

(2R)-hydroxy (4-nitrophenyl) acetic acid (150.0 mg, 0.76 mmol), m-PEG24-amine (909.4 mg, 0.84 mmol) and HATU (346.4 mg, 0.91 mmol) were dissolved in anhydrous DMF (7.0 mL) under room temperature. NMM (0.25 mL, 2.30 mmol) was added. The resulting mixture was stirred at room temperature for 6-8 hours. 7% NaHCO_{3(aq)} (7.0 mL) was then added, and the mixture solution was extracted with CH₂Cl₂ (10 mL×3). The combined organic layers were dried with MgSO₄ and then concentrated in vacuo at 30-35° C. The crude H-PAB-1 was dried in high vacuum and then used in the next step without further purification.

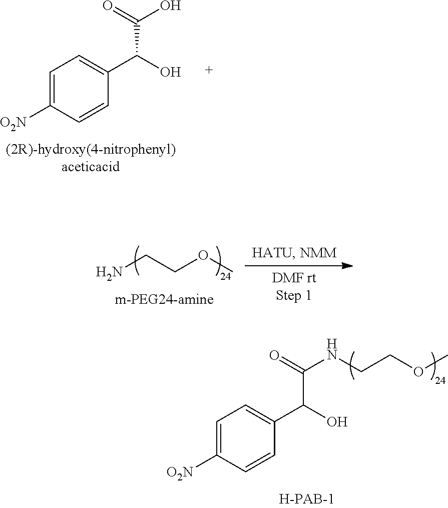

### Step 2:

To a solution of crude H-PAB-1 in EA/MeOH (25 mL, 95/5), 10% Pd/C (165.0 mg, 5 wt %) was added. The reaction was stirred at room temperature with H₂ balloon for 24 hours. After the reaction was completed, the reaction mixture was passed through celite (3.3 g) and the celite was washed with MeOH (10 mL×3). The desired fraction was concentrated in vacuo at 30-35° C. The crude H-PAB-2 was dried in high vacuum and then used in the next step without further purification. HRMS (ESI) m/z found [M+H]⁺, 1237.7278 C₅₇H₁₀₉N₂O₂₆⁺, required 1237.7269.

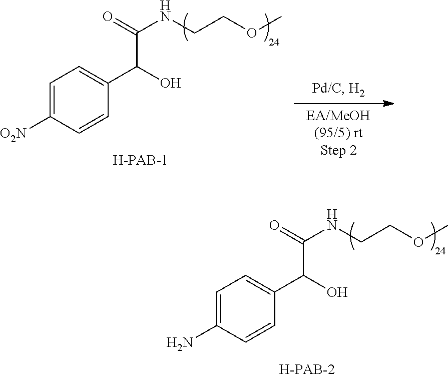

### Step 3:

To a solution of crude H-PAB-2 in anhydrous CH₂Cl₂ (25 mL), Boc-VA-OH (792.8 mg, 2.75 mmol) and EEDQ (741.8 mg, 3.00 mmol) were added. The mixture was stirred at room temperature for 18-24 hours. After the reaction was completed, 1 M HCl_{(aq)} (15 mL) was added and then the mixture solution was extracted with CH₂Cl₂ (15 mL×3). The combined organic layers were washed with H₂O (20 mL) and then concentrated in vacuo at 30-35° C. The resulting mixture was purified with preparative HPLC to obtain 440.0 mg of H-PAB-3 with 38.4% yield for three steps. HRMS (ESI) m/z found [(M+2Na)/2]⁺, 776.4278 C₇₀H₁₃₀N₄O₃₀Na₂₂⁺, required 776.4282.

TABLE 3
HPLC condition (Column:
YMC-Actus Trait C18 250 × 20 mm, 5 μm, 12 nm)

| Gradient: | | | |
|---|---|---|---|
| Time | Flow (mL/min) | H₂O | ACN |
| 0 min | 10 | 90% | 10% |
| 1 min | 20 | 75% | 25% |
| 6 min | 20 | 65% | 35% |
| 8 min | 20 | 65% | 35% |
| 10 min | 20 | 63% | 37% |
| 12 min | 20 | 63% | 37% |
| 14 min | 20 | 61% | 39% |
| 15 min | 20 | 50% | 50% |
| 15.1 min | 20 | 10% | 90% |
| 21 min | 20 | 10% | 90% |

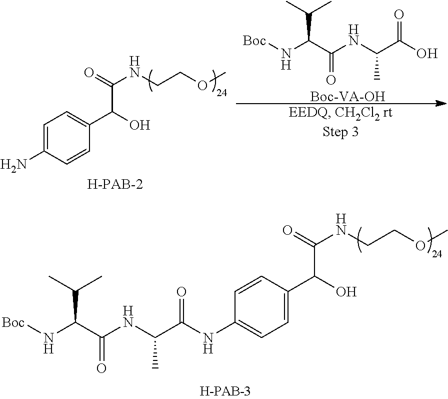

### Steps 4 and 5:

H-PAB-3 (440.0 mg, 0.29 mmol) and Bis(4-nitrophenyl) carbonate (177.5 mg, 0.58 mmol) in anhydrous CH₂Cl₂ (5.8 mL) was cooled to 0-4° C. 2,6-lutidine (51.0 μL, 0.43 mmol) and DIPEA (50.9 μL, 0.29 mmol) were added sequentially at 0-4° C. The resulting mixture was stirred at 0-4° C. and monitored by HPLC. After H-PAB-3 was consumed, Et₂NH (6.0 μL×5) was added separately at 0-4° C. to quench excess Bis(4-nitrophenyl) carbonate. Anhydrous DMF (11.6 ml), exatecan mesylate (278.4 mg, 0.52 mmol), and DIPEA (188.0 μL, 1.07 mmol) were added into the reaction solution at 0-4° C. The reaction mixture was stirred at 0-4° C. until H-PAB-4 was consumed by checking with HPLC. After the reaction was completed, the resulting mixture was purified with preparative HPLC to obtain 229.8 mg of H-PAB-5 with 40.0% yield. HRMS (ESI) m/z found [(M+2H)/2]⁺, 985.0144 C₉₅H₁₅₂FN₇O₃₅²⁺, required 985.0156.

TABLE 4
HPLC condition (Column:
YMC-Actus Trait C18 250 × 20 mm, 5 μm, 12 nm)

| Gradient: | | | |
|---|---|---|---|
| Time | Flow (mL/min) | H₂O | ACN |
| 0 min | 10 | 90% | 10% |
| 1 min | 20 | 70% | 30% |
| 3 min | 20 | 55% | 45% |
| 4 min | 20 | 54% | 46% |
| 6 min | 20 | 53% | 47% |
| 8 min | 20 | 52% | 48% |
| 10 min | 20 | 52% | 48% |
| 14 min | 20 | 50% | 50% |
| 15 min | 20 | 46% | 54% |
| 16 min | 20 | 42% | 58% |
| 17 min | 20 | 38% | 62% |
| 17.1 min | 20 | 10% | 90% |
| 22 min | 20 | 10% | 90% |

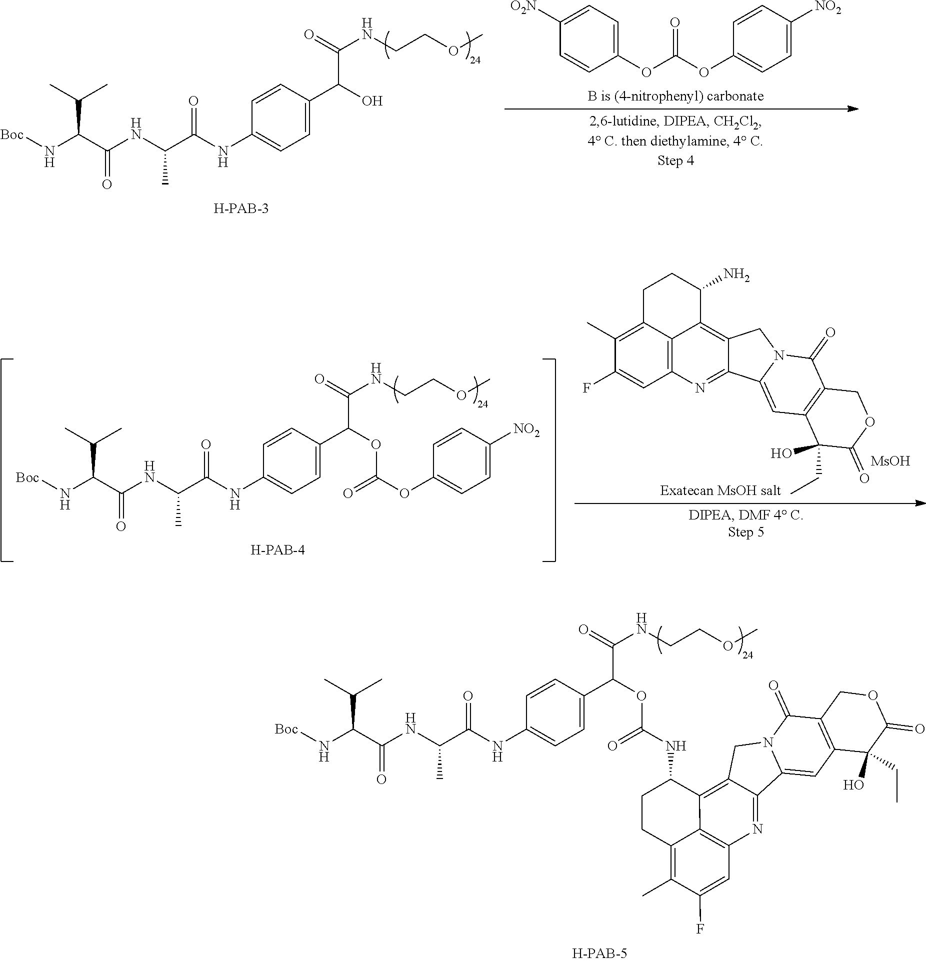

### Step 6:

A solution of H-PAB-5 (45.0 mg, 0.02 mmol) in CH₂Cl₂ (0.9 mL) was cooled to 0° C. TFA (225.0 0 μL) was added dropwise. The reaction mixture was stirred at 0° C. for 4-6 hours. After the reaction was completed, the reaction mixture was added dropwise into a stirring ether (11.3 mL) to get precipitate. The solids were collected by filtration and washed with ether (5 mL×3), and followed by dried in high vacuum to obtain crude H-PAB-6. The crude product was used in the next step without further purification.

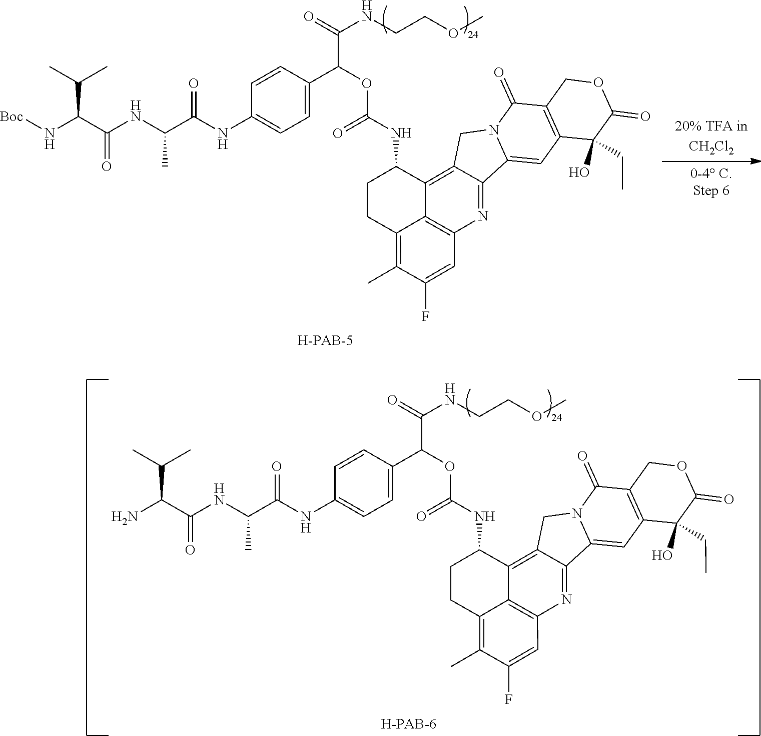

### Step 7:

To a solution of crude H-PAB-6 (0.023 mmol) in anhydrous DMF (0.45 mL), HATU (10.4 mg, 0.027 mmol) and DBCO-acid (6.9 mg, 0.023) were added and the reaction mixture was cooled to 0-4° C. After 5 minutes, Et₃N (9.62 μL, 0.069 mmol) was added slowly. The reaction mixture was stirred at 0-4° C. for 4 hours. After the reaction was completed, the resulting mixture was purified with preparative HPLC to obtain 27.6 mg of DL-5 with 56.2% yield. HRMS (ESI) m/z found [(M+2H)/2]⁺, 1078.5447 C₁₀₉H₁₅₇FN₈O₃₅²⁺, required 1078.5367.

TABLE 5
HPLC condition (Column:
YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 70% | 30% |
| 3 min | 20 | 55% | 45% |
| 4 min | 20 | 52% | 48% |
| 6 min | 20 | 50% | 50% |
| 8 min | 20 | 48% | 52% |
| 10 min | 20 | 46% | 54% |
| 14 min | 20 | 46% | 54% |
| 15 min | 20 | 45% | 55% |
| 16 min | 20 | 40% | 60% |
| 17 min | 20 | 10% | 90% |
| 21 min | 20 | 10% | 90% |

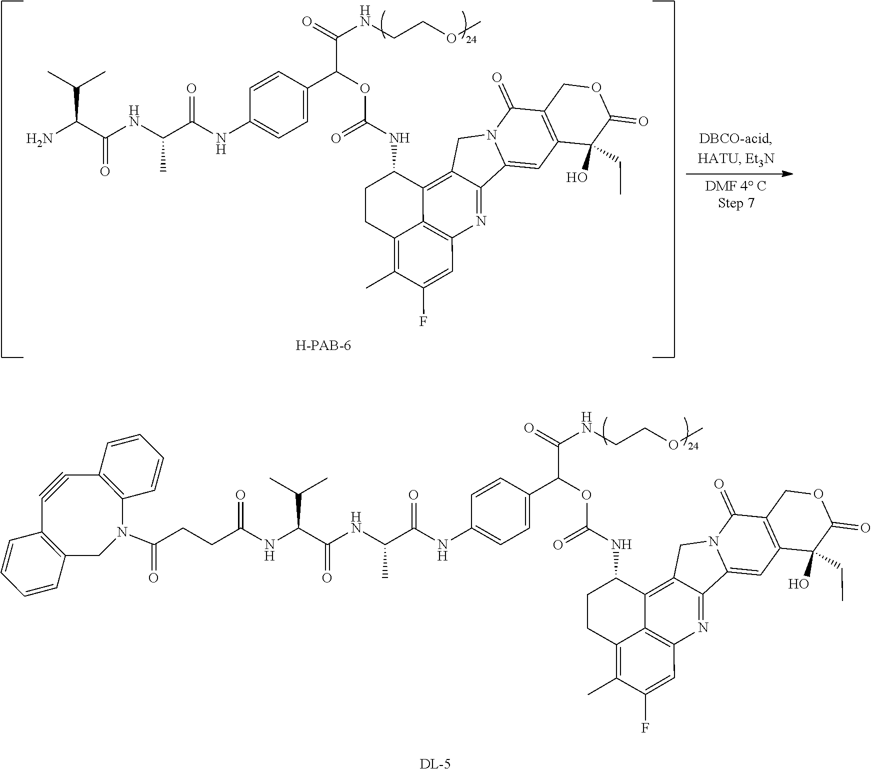

### 3-5. Preparation of BCN-VA-Hydra-PAB-PEG₂₄-Exatecan (Also Termed DL-6)

A solution of H-PAB-5 (36.8 mg, 0.0187 mmol) in CH₂Cl₂ (0.74 mL) was cooled to 0° C. TFA (184.0 μL) was added dropwise. The reaction mixture was stirred at 0° C. for 4 hours. After the reaction was completed, the reaction mixture was diluted with MeOH (5 mL) and then concentrated in vacuo at 30-35° C. and then azeotroped with toluene (5 mL×3) to remove excess TFA. The resulting material was dried in high vacuum to obtain crude deprotected intermediate. A solution of the crude intermediate (0.0187 mmol) in anhydrous DMF (0.37 mL), BCN—Osu (5.4 mg, 0.0187 mmol) and Et₃N (7.8 μL, 0.0561 mmol) were added sequentially. The reaction mixture was stirred at room temperature overnight. After the reaction was completed, the resulting mixture was purified with preparative HPLC to obtain 23.8 mg of DL-6 with 62.2% yield. HRMS (ESI) m/z found [(M+2H)/2]⁺, 1023.0380 C₁₀₁H₁₅₄FN₇O₃₅²⁺, required 1023.0235.

TABLE 6
HPLC condition (Column:
YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 70% | 30% |
| 5 min | 20 | 55% | 45% |
| 25 min | 20 | 45% | 55% |
| 26 min | 20 | 10% | 90% |
| 33 min | 20 | 10% | 90% |

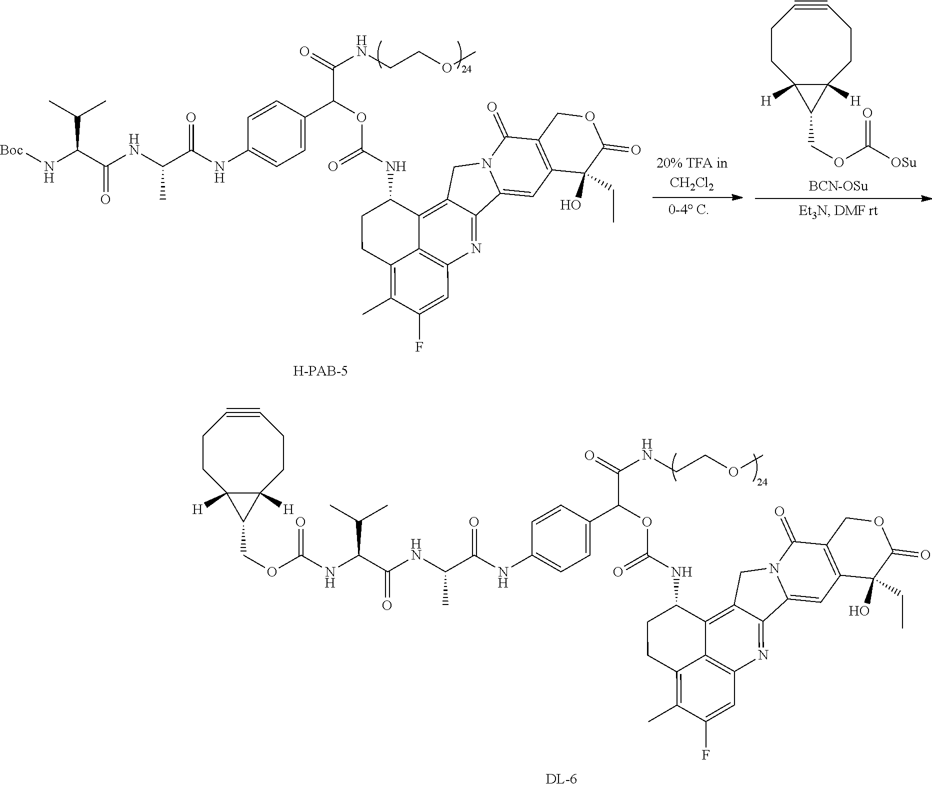

### 3-6. Preparation of DBCO-GGVA-Hydra-PAB-PEG₂₄-Exatecan (Also Termed DL-7)

### Step 1:

DBCO-acid (265.7 mg, 0.87 mmol), tert-butyl 2-(2-aminoacetamido)acetate (163.8 mg, 0.87 mmol) and HATU (363.9 mg, 0.96 mmol) were dissolved in CH₂Cl₂ (8.7 mL) under room temperature. NMM (287.0 μL, 2.61 mmol) was added. After the addition, the resulting mixture was stirred at room temperature for 16-18 hours. The reaction solution was concentrated in vacuo at 30-35° C., and the residue was purified by a flash silica gel column (CH₂Cl₂/EA=1/2 to 1/4) to obtain 342.2 mg of DBCO-4 with 82.7% yield.

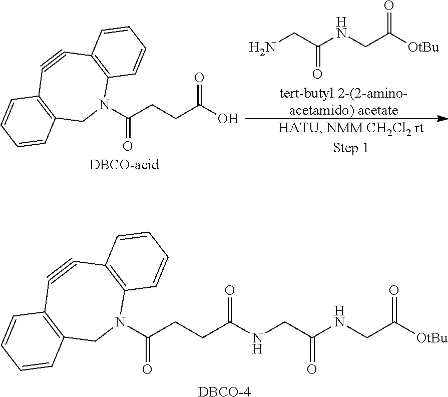

### Step 2:

A solution of DBCO-4 (340.0 mg, 0.71 mmol) in CH₂Cl₂ (6.8 mL) was cooled to 0° C. TFA (1.7 mL) was added dropwise. The reaction mixture was warmed to room temperature (rt) slowly and then stirred for 1-2 hours. After the reaction was completed, the reaction mixture was concentrated in vacuo at 30-35° C. and then azeotroped with toluene (5 mL×3) to remove excess TFA. The resulting mixture was purified with preparative HPLC to obtain 168.4 mg of DBCO-5 with 56.1% yield.

TABLE 7
HPLC condition (Column:
YMC-Actus Trait C18 250 × 20 mm, 5 μm, 12 nm)

| Gradient: | | | |
|---|---|---|---|
| Time | Flow (ml/min) | H₂O | ACN |
| 0 min | 20 | 90% | 10% |
| 2 min | 20 | 80% | 20% |
| 10 min | 20 | 60% | 40% |
| 20 min | 20 | 60% | 40% |
| 25 min | 20 | 10% | 90% |
| 27 min | 20 | 10% | 90% |

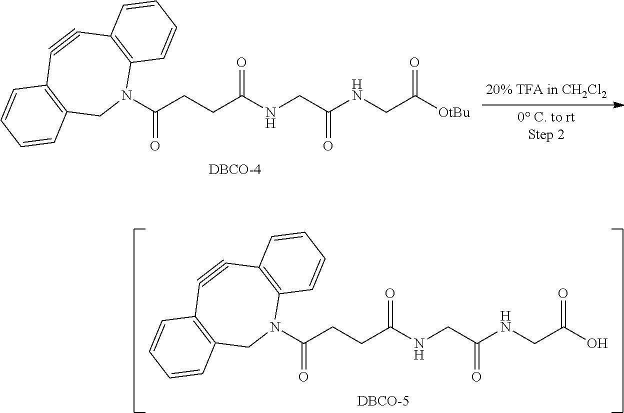

### Step 3:

DBCO-5 (3.98 mg, 0.0095 mmol), H-PAB-6 (17.8 mg, 0.0095 mmol) and HATU (3.97 mg, 0.0104 mmol) were dissolved in anhydrous DMF (0.32 mL). Et₃N (4.0 μL, 0.0285 mmol) was added. The reaction mixture was stirred at room temperature for 3-4 hours. After the reaction was completed, the resulting mixture was purified with preparative HPLC to obtain 10.7 mg of DL-7 with 49.6% yield.

TABLE 8
HPLC condition (Column:
YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 80% | 20% |
| 5 min | 20 | 60% | 40% |
| 15 min | 20 | 45% | 55% |
| 20 min | 20 | 10% | 90% |
| 25 min | 20 | 10% | 90% |

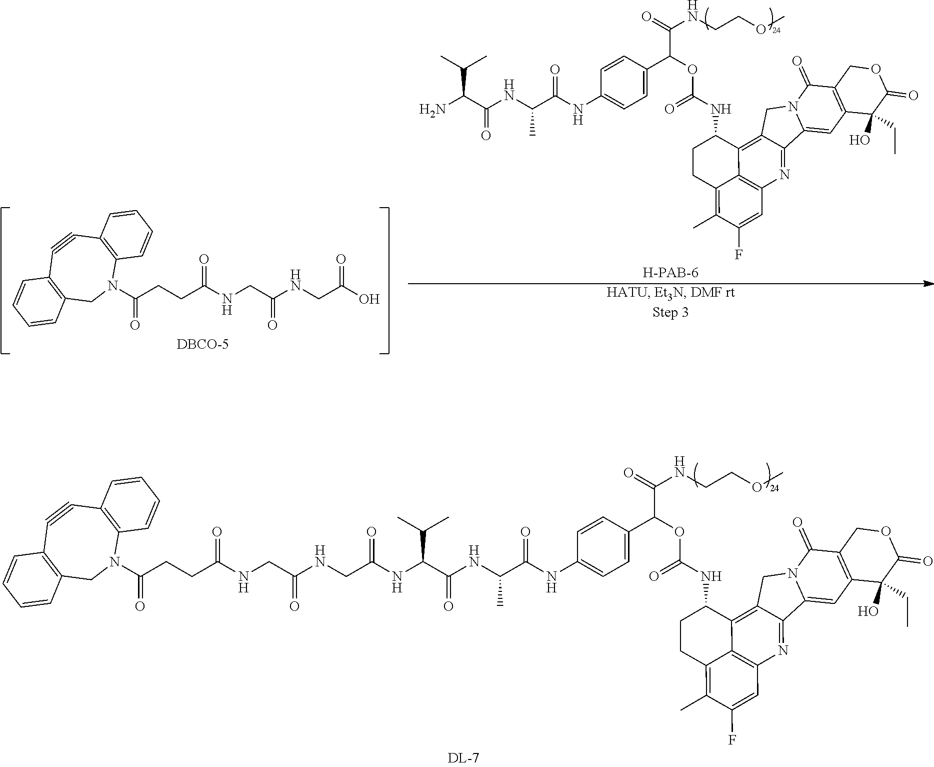

### 3-7. Preparation of BCN-GGVA-Hydra-PAB-PEG₂₄-Exatecan (Also Termed DL-8)

### Step 1:

A solution of H-PAB-5 (57.0 mg, 0.0289 mmol) in CH₂Cl₂ (1.2 mL) was cooled to 0° C. TFA (285.0 μL) was added dropwise. The reaction mixture was stirred at 0-4° C. for 3-4 hours. After the reaction was completed, the reaction mixture was diluted with MeOH (2 mL). The solution was concentrated in vacuo at 30-35° C. and then azeotroped with toluene (5 mL×3) to remove excess TFA. The residue was dried in high vacuum to generate crude deprotected intermediate. The prepared crude intermediate, Boc-Gly-OH (5.1 mg, 0.0289 mmol) and HATU (13.2 mg, 0.0347 mmol) were dissolved in anhydrous DMF (0.58 mL). Et₃N (12.1 μL, 0.0868 mmol) was added. The reaction mixture was stirred at room temperature overnight. After the reaction was completed, the resulting mixture was purified with preparative HPLC to obtain 40.4 mg of H-PAB-7 with 68.9% yield.

TABLE 9
HPLC condition (Column:
YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 80% | 20% |
| 5 min | 20 | 60% | 40% |
| 25 min | 20 | 50% | 50% |
| 28 min | 20 | 10% | 90% |
| 33 min | 20 | 10% | 90% |

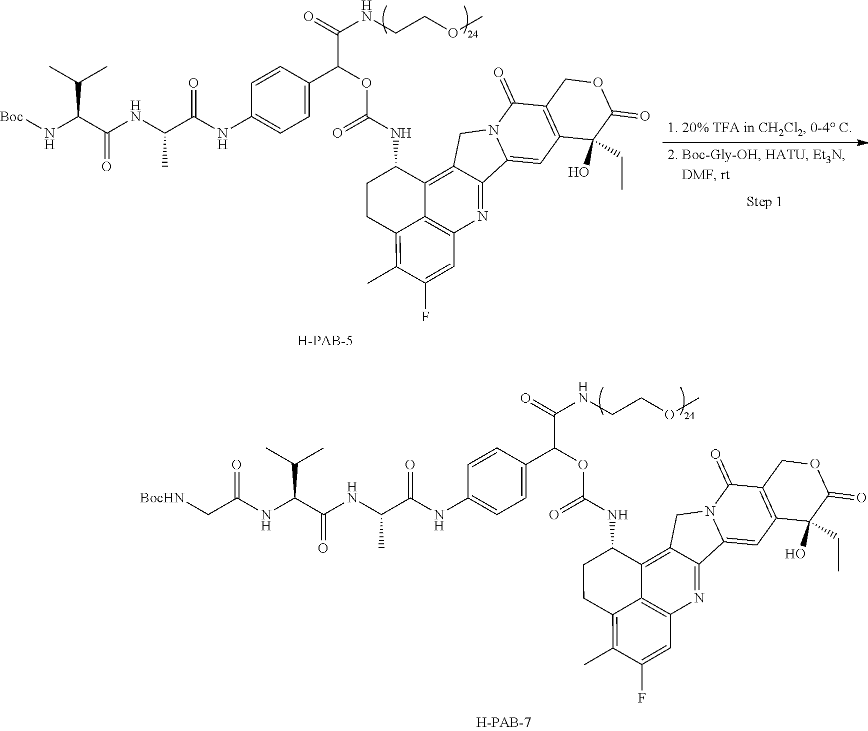

### Step 2:

A solution of H-PAB-7 (38.5 mg, 0.0190 mmol) in CH₂Cl₂ (0.77 mL) was cooled to 0° C. TFA (192.5 μL) was added dropwise. The reaction mixture was stirred at 0-4° C. for 3-4 hours. After the reaction was completed, the reaction mixture was diluted with MeOH (2 mL). The solution was concentrated in vacuo at 30-35° C. and then azeotroped with toluene (5 mL×3) to remove excess TFA. The residue was dried in high vacuum to generate crude deprotected intermediate. The prepared crude intermediate, Boc-Gly-OH (3.4 mg, 0.0190 mmol) and HATU (8.7 mg, 0.0228 mmol) were dissolved in anhydrous DMF (0.38 mL). Et₃N (7.9 μL, 0.0570 mmol) was added. The reaction mixture was stirred at room temperature overnight. After the reaction was completed, the resulting mixture was purified with preparative HPLC to obtain 28.7 mg of H-PAB-8 with 72.6% yield.

TABLE 10
HPLC condition (Column:
YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 70% | 30% |
| 3 min | 20 | 55% | 45% |
| 4 min | 20 | 54% | 46% |
| 6 min | 20 | 53% | 47% |
| 8 min | 20 | 52% | 48% |
| 10 min | 20 | 52% | 48% |
| 14 min | 20 | 50% | 50% |
| 15 min | 20 | 46% | 54% |
| 16 min | 20 | 42% | 58% |
| 17 min | 20 | 38% | 62% |
| 17.1 min | 20 | 10% | 90% |
| 22 min | 20 | 10% | 90% |

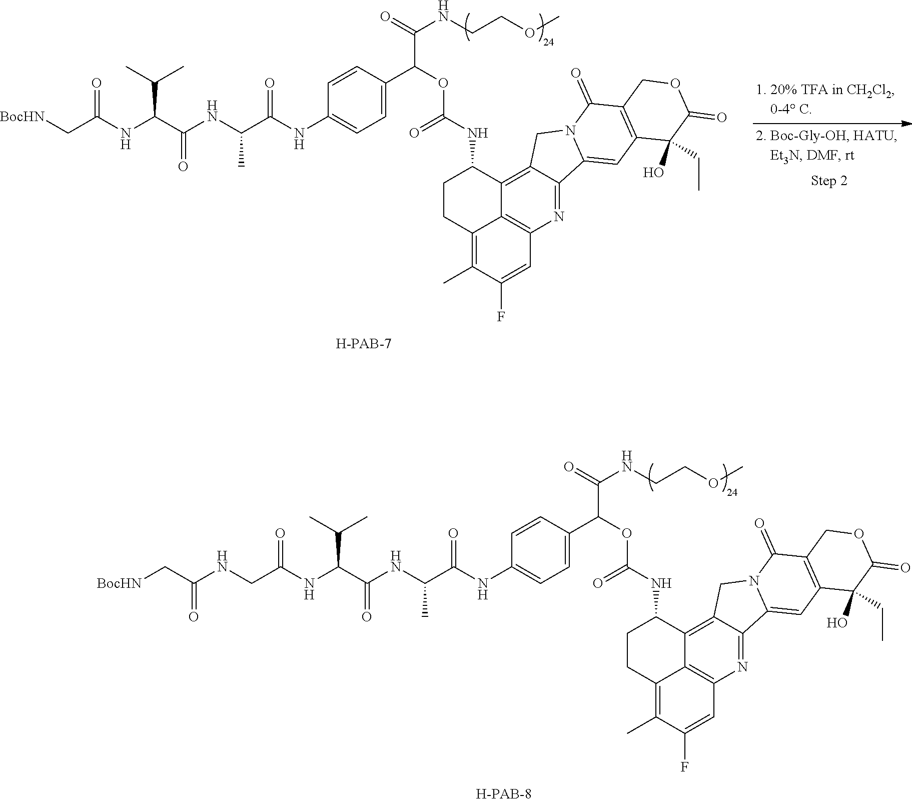

### Step 3:

A solution of H-PAB-8 (25.0 mg, 0.0120 mmol) in CH₂Cl₂ (0.50 mL) was cooled to 0° C. TFA (125.0 μL) was added dropwise. The reaction mixture was stirred at 0° C. for 4 hours. After the reaction was completed, the reaction mixture was diluted with MeOH (5 mL) and then concentrated in vacuo at 30-35° C. and then azeotroped with toluene (5 mL×3) to remove excess TFA. The resulting material was dried in high vacuum to obtain crude deprotected intermediate. To a solution of the crude intermediate (0.0120 mmol) in anhydrous DMF (0.24 mL), BCN—OSu (3.5 mg, 0.0120 mmol) and Et₃N (5.1 μL, 0.0360 mmol) were added sequentially. The reaction mixture was stirred at room temperature overnight. After the reaction was completed, the resulting mixture was purified with preparative HPLC to obtain 15.7 mg of DL-8 with 60.9% yield.

TABLE 11
HPLC condition (Column:
YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 70% | 30% |
| 3 min | 20 | 55% | 45% |
| 4 min | 20 | 54% | 46% |
| 6 min | 20 | 53% | 47% |
| 8 min | 20 | 52% | 48% |
| 10 min | 20 | 52% | 48% |
| 14 min | 20 | 50% | 50% |
| 15 min | 20 | 46% | 54% |
| 16 min | 20 | 42% | 58% |
| 17 min | 20 | 38% | 62% |
| 17.1 min | 20 | 10% | 90% |
| 22 min | 20 | 10% | 90% |

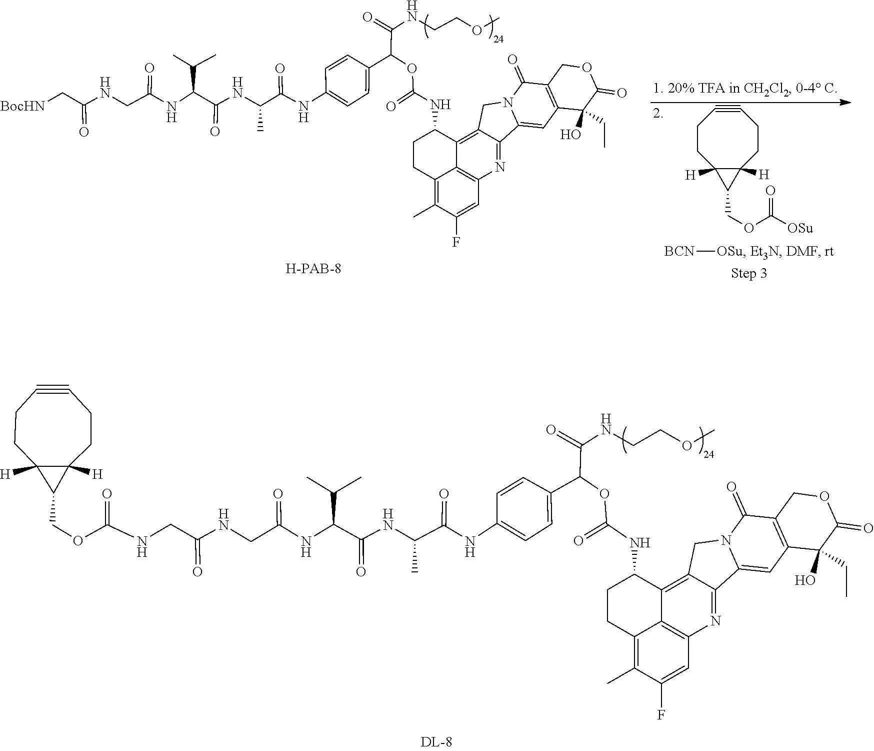

### 3-8. Preparation of BCN-GGVA-Hydra-PAB-MMAE (Also Termed DL-14) and BCN-GGVA-Hydra-PAB-T785 (Also Termed DL-15)

### Step 1:

To a solution of N-DT-0024 (50.0 mg, 0.065 mmol) and payload (MMAE or T785, 1.1 eq) in anhydrous DMF (0.65 mL), DIPEA (33.4 μL, 0.192 mmol) was added slowly. The resulting mixture was stirred at room temperature until N-DT-0024 was consumed. After the reaction was completed, the reaction mixture was added dropwise into a stirring TBME (6.5 mL) to get precipitate. The solids were collected by filtration and followed with high vacuum drying to obtain crude HL-1 (X=MMAE with 75.6% yield) or HL-2 (X=T785 with 79.1% yield). This crude product was used in the next step without further purification.

### Step 2:

A stirring suspension of HL-1 or HL-2 (83.4 mg) in DCM (1.46 mL) was cooled to 0° C. TFA (0.63 mL) was added at 0° C. slowly. The reaction mixture was stirred at 0-4° C. for 20-24 hours. After the reaction was completed, the reaction mixture was added dropwise into a stirring TBME (20.9 mL) to get precipitate. The solids were collected by filtration and followed with high vacuum drying to obtain crude intermediate. This crude product was used in the next step without further purification.

The intermediate and BCN—OSu (1 eq) were dissolved in anhydrous DMF (0.05 M). Et₃N (3 eq) was added into the reaction solution at room temperature. The reaction mixture was stirred at room temperature overnight. After the reaction was completed, the reaction mixture was added dropwise into a stirring TBME (10 times amount of intermediate) to get precipitate. The solids were collected by filtration and followed with high vacuum drying to obtain crude BCN intermediate. This crude product was used in the next step without further purification.

The BCN intermediate (1 eq), NH₂-PEG₂₄-Ome (2 eq) and HATU (1.2 eq) were dissolved in anhydrous DMF (0.05 M). NMM (5 eq) was added. The reaction mixture was stirred at room temperature overnight. After the reaction was completed, the resulting mixture was purified with preparative HPLC to obtain DL-14 or DL-15. DL-14 was obtained with 26.0% yield. MS (ESI) m/z found [(M+H+NH₄/2]⁺, 1229.810 C₁₂₀H₂₁₀N₁₂O₄₀²⁺, required 1229.738. DL-15 was obtained with 27.7% yield. HRMS (ESI) m/z found [(M+2H)/2]⁺, 1018.0743 C₉₉H₁₆₅N₁₁O₃₃²⁺, required 1018.0785.

TABLE 12
HPLC condition (Column:
YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 90% | 10% |
| 5 min | 20 | 80% | 20% |
| 20 min | 20 | 60% | 40% |
| 25 min | 20 | 10% | 90% |
| 30 min | 20 | 10% | 90% |

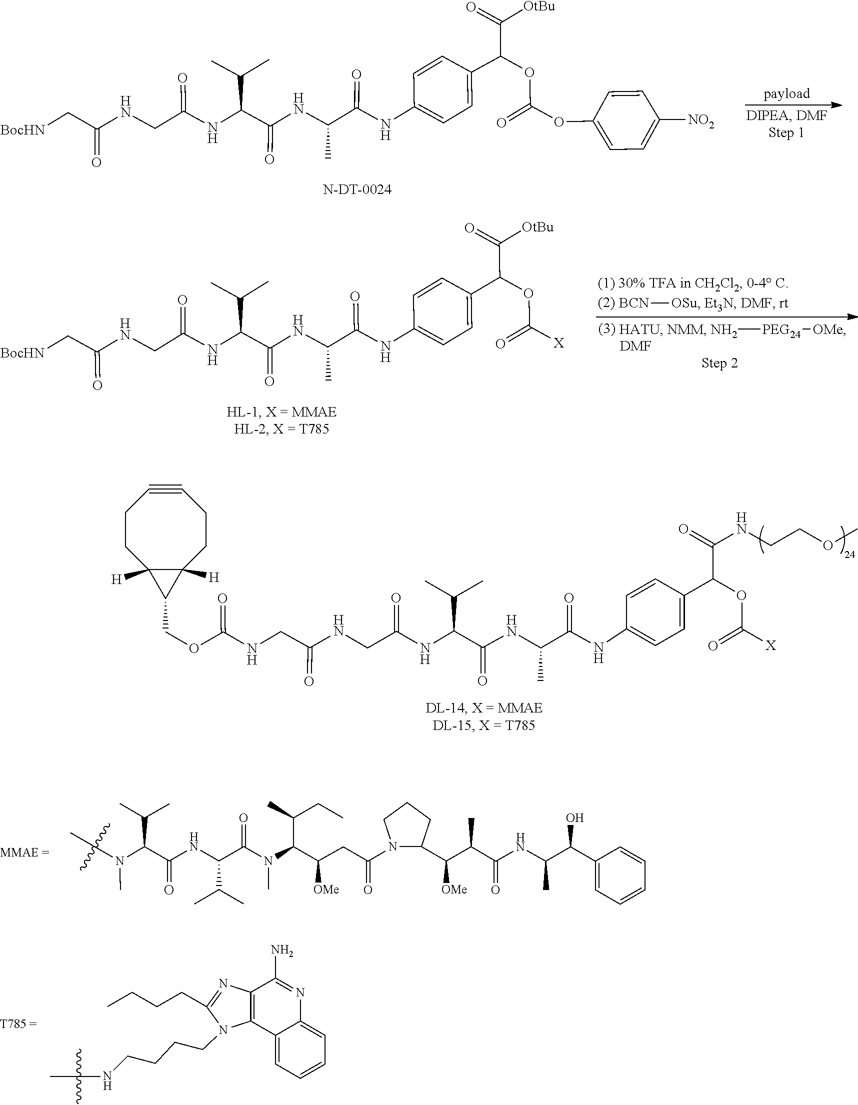

### 3-9. Preparation of BCN-GGVA-Hydra-PAB-Eribulin (Also Termed DL-16)

### Step 1:

Fmoc-VA-PAB (COOtBu)-OH (202.8 mg, 0.329 mmol) in anhydrous CH₂Cl₂ (3.8 mL) was cooled to 0° C. After five minutes, TFA (1.27 mL) was added slowly. The reaction mixture was stirred at 0-4° C. overnight. After the reaction was completed, the reaction mixture was added dropwise into a stirring TBME (50.0 mL) to get precipitate. The solids were collected by filtration and followed with high vacuum drying to obtain a crude intermediate (75.71 mg). The previous intermediate (75.71 mg, 0.135 mmol), NH₂-PEG₂₄-OMe (161.9 mg, 0.149 mmol) and HATU (61.7 mg, 0.162 mmol) were dissolved in anhydrous DMF (1.35 mL). NMM (44.8 μL, 0.406 mmol) was added and the reaction mixture was stirred at room temperature for 6-8 hours. After the reaction was completed, the reaction mixture was concentrated in vacuo at 30-35° C., and the residue was purified by a flash silica gel column (CH₂Cl₂/MeOH=12/1 to 9/1) to obtain 212.7 mg of HL-3 with 39.6% yield.

### Step 2:

HL-3 (22.57 mg, 0.014 mmol) and Bis(4-nitrophenyl) carbonate (8.42 mg, 0.028 mmol) in anhydrous CH₂Cl₂ (0.28 mL) was cooled to 0-4° C. 2,6-lutidine (2.4 μL, 0.021 mmol) and DIPEA (2.4 μL, 0.014 mmol) were added sequentially at 0-4° C. The resulting mixture was stirred at 0-4° C. for 20-24 hours. Et₂NH (1.4 μL, 0.014 mmol) was added slowly at 0-4° C. to quench excess Bis(4-nitrophenyl) carbonate. Anhydrous DMF (0.55 mL), Eribulin (15.2 mg, 0.021 mmol), DIPEA (7.2 μL, 0.042 mmol) were added into previous reaction solution at 0-4° C. The reaction mixture was stirred at 0-4° C. overnight. After the reaction was completed, the resulting mixture was purified with preparative HPLC to obtain 7.54 mg of HL-4 with 22.8% yield.

TABLE 13
HPLC condition (Column:
YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 80% | 20% |
| 15 min | 20 | 50% | 50% |
| 25 min | 20 | 10% | 90% |
| 35 min | 20 | 10% | 90% |

### Step 3:

HL-4 (7.54 mg, 0.003 mmol) was dissolved in CH₂Cl₂/MeOH (226.0 μL, 1/1) and then cooled to 0° C. Et₂NH (45.2 μL) was added and the reaction mixture was stirred at 0-4° C. overnight. After the reaction was completed, the reaction mixture was concentrated in vacuo at 30-35° C. and azeotroped with toluene (1 mL×3) to remove excess Et₂NH. The crude compound was dried in high vacuum to obtain a crude intermediate (8.2 mg). The intermediate (8.2 mg), Fmoc-GG-OH (1.61 mg, 0.004 mmol) and HATU (2.16 mg, 0.005 mmol) were dissolved in anhydrous DMF (0.2 mL). NMM (1.25 μL, 0.011 mmol) was added at room temperature. The resulting mixture was stirred at room temperature overnight. After the reaction was completed, the reaction mixture was concentrated in vacuo at 30-35° C., and the residue was purified by a flash silica gel column (CH₂Cl₂/MeOH=100/0 to 87/13) to obtain Fmoc-GG-intermediate. Fmoc-GG-intermediate was dissolved in CH₂Cl₂/MeOH (0.6 mL, 1/1) and then cooled to 0° C. Et₂NH (0.13 mL) was added and the reaction mixture was stirred at 0-4° C. for 20-24 hours. After the reaction was completed, the reaction mixture was concentrated in vacuo at 30-35° C. and azeotroped with toluene (2 mL×3) to remove excess Et₂NH. The crude compound was dried in high vacuum to obtain a crude intermediate. The intermediate (19.3 mg), BCN—OSu (2.51 mg, 0.008 mmol) were dissolved in anhydrous DMF 0.17 mL. Et₃N (3.61 μL, 0.026 mmol) was added. The resulting mixture was stirred at room temperature overnight. After the reaction was completed, the resulting mixture was purified with preparative HPLC to obtain 2.85 mg of DL-16 with 37.4% yield. HRMS (ESI) m/z found [(M+2H)/2]⁺, 1227.1713 C₁₂₁H₁₉₉N₇O₄₄²⁺, required 1227.1697.

TABLE 14
HPLC condition (Column:
YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 80% | 20% |
| 15 min | 20 | 50% | 50% |
| 25 min | 20 | 10% | 90% |
| 35 min | 20 | 10% | 90% |

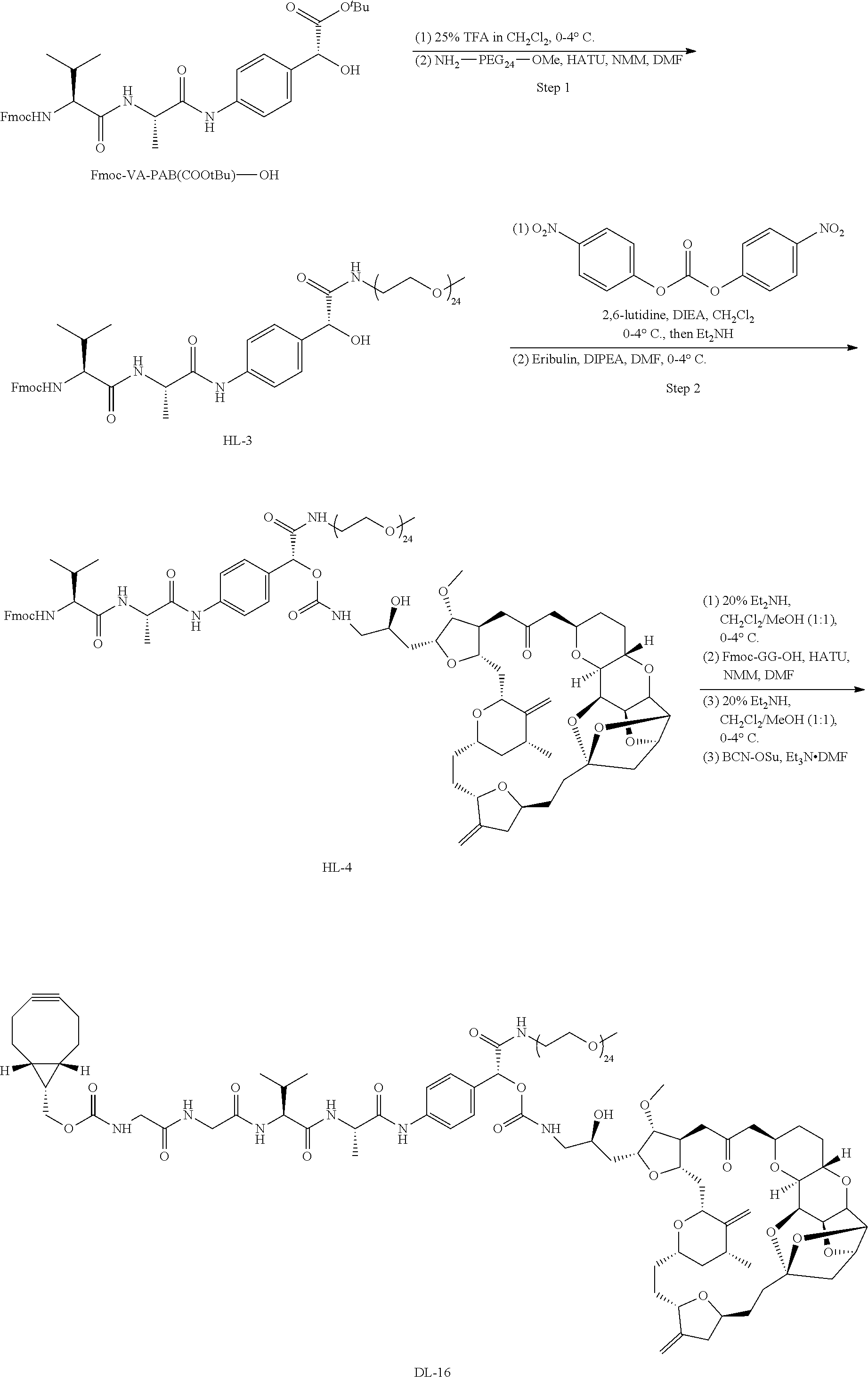

### 3-10. Preparation of BCN-Glu (PEG₂₄)-VA-Hydra-PAB-Exatecan (Also Termed DL-17)

### Step 1:

H-PAB-6 (57.3 mg, 0.029 mmol), Fmoc-Glu (PEG₂₄) (43.7 mg, 0.030 mmol) and HATU (22.7 mg, 0.060 mmol) were dissolved in anhydrous DMF (0.29 mL). NMM (9.5 μL, 0.087 mmol) was added and the reaction mixture was stirred at room temperature for 1-2 hours. After the reaction was completed, the resulting mixture was purified with preparative HPLC to obtain 72.6 mg of HL-5 with 76.1% yield. HRMS (ESI) m/z found [(M+4H)/4]⁺, 823.1910 C₁₅₉H₂₆₂FN₉O₆₁⁴⁺, required 823.1915.

TABLE 15
HPLC condition (Column:
YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 80% | 20% |
| 5 min | 20 | 70% | 30% |
| 8 min | 20 | 62% | 38% |
| 30 min | 20 | 48% | 52% |
| 30.1 min | 20 | 10% | 90% |
| 34 min | 20 | 10% | 90% |
| 35 min | 20 | 10% | 90% |

### Step 2:

HL-5 (70.4 mg, 0.021 mmol) was dissolved in CH₂Cl₂/MeOH (1.69 mL, 1/1) and then cooled to 0° C. Et₂NH (0.42 mL) was added and the reaction mixture was stirred at 0-4° C. for 8-10 hours. After the reaction was completed, the reaction mixture was concentrated in vacuo at 30-35° C. and azeotroped with toluene (5 mL×3) to remove excess Et₂NH. This crude product HL-6 was used in the next step without further purification.

### Step 3:

HL-6 (38.6 mg, 0.013 mmol) and BCN—OSu (3.7 mg, 0.013 mmol) were dissolved in anhydrous DMF 0.26 mL. Et₃N (5.3 μL, 0.038 mmol) was added. The resulting mixture was stirred at room temperature for 1-2 hours. After the reaction was completed, the resulting mixture was purified with preparative HPLC to obtain 27.8 mg of DL-17 with 65.9% yield. HRMS (ESI) m/z found [(M+3Na)/3]⁺, 1103.9109 C₁₅₅H₁₆₀FN₉O₆₁Na₃³⁺, required 1103.9065.

TABLE 16
HPLC condition (Column:
YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 80% | 20% |
| 5 min | 20 | 70% | 30% |
| 8 min | 20 | 62% | 38% |
| 30 min | 20 | 48% | 52% |
| 30.1 min | 20 | 10% | 90% |
| 34 min | 20 | 10% | 90% |
| 35 min | 20 | 10% | 90% |

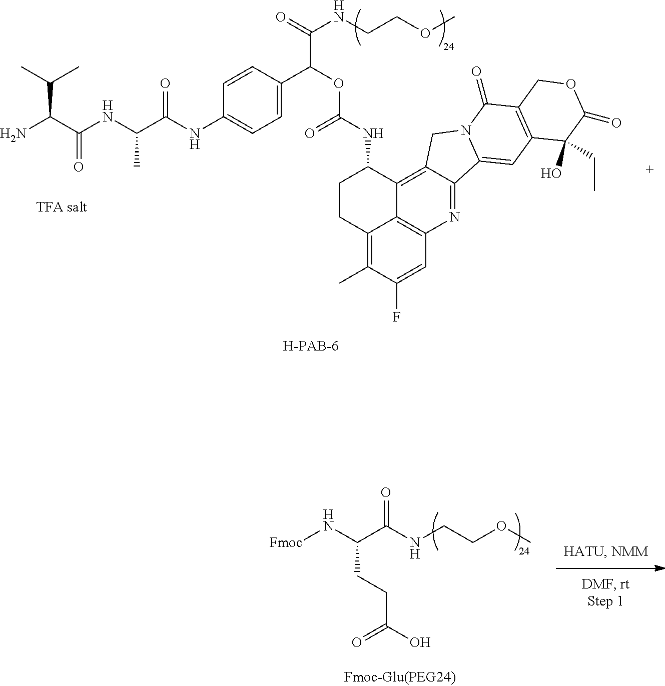

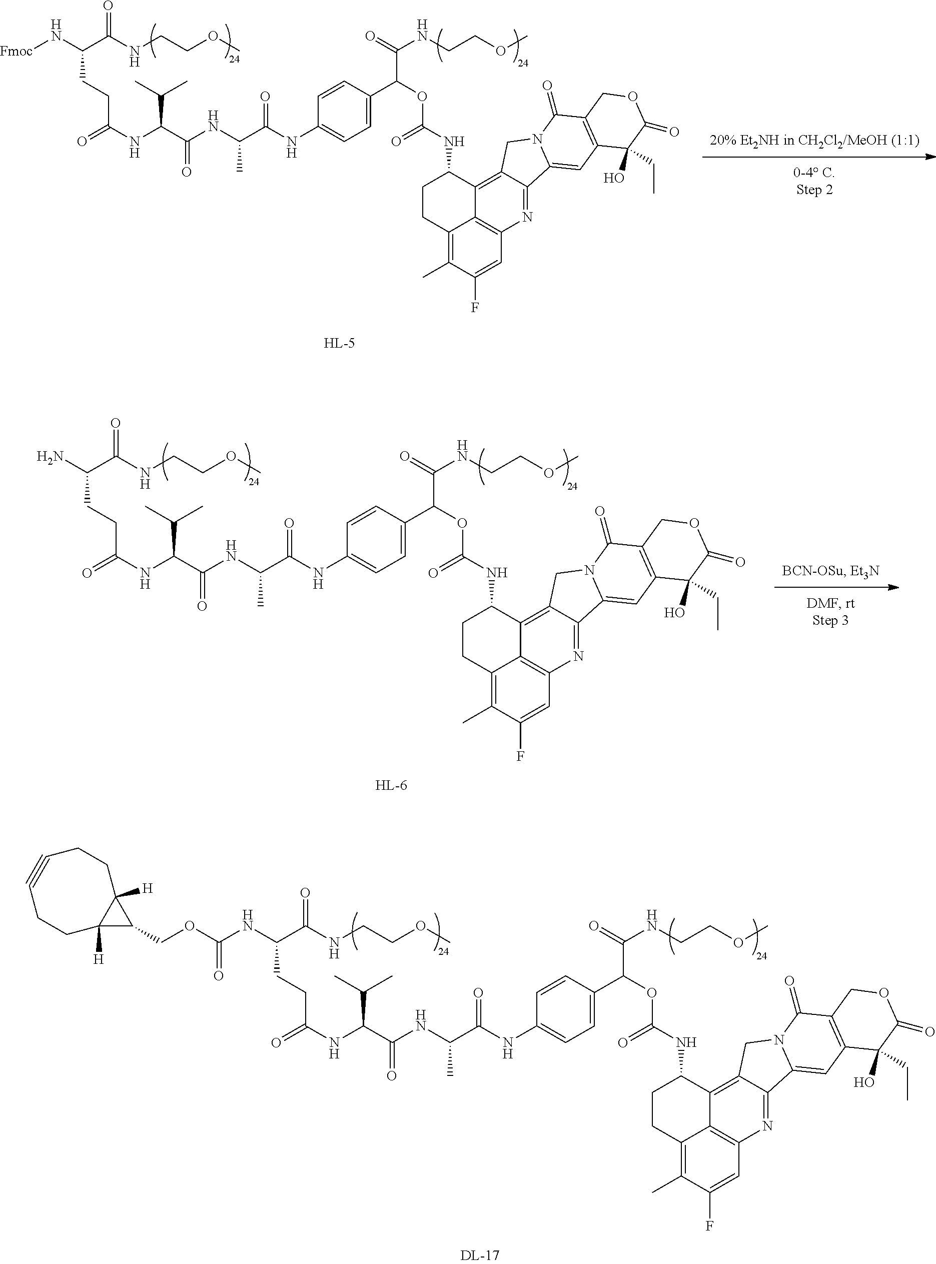

### 3-11. Preparation of DBCO-Glu (PEG₂₄)-VA-Hydra-PAB-Exatecan (Also Termed DL-18)

HL-6 (15.2 mg, 0.0050 mmol), DBCO-acid (1.5 mg, 0.0050 mmol) and HATU (2.83 mg, 0.0074 mmol) were dissolved in anhydrous DMF (0.10 mL). Et₃N (2.1 μL, 0.0149 mmol) was added. The reaction mixture was stirred at room temperature for 1-2 hours. After the reaction was completed, the resulting mixture was purified with preparative HPLC to obtain 10.2 mg of DL-18 with 61.6% yield. HRMS (ESI) m/z found [(M+3H)/3]⁺, 1118.9224 C₁₆₃H₂₆₄FN₁₀O₆₁³⁺, required 1118.9282.

TABLE 17
HPLC condition (Column:
YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 80% | 20% |
| 5 min | 20 | 70% | 30% |
| 8 min | 20 | 62% | 38% |
| 30 min | 20 | 48% | 52% |
| 30.1 min | 20 | 10% | 90% |
| 34 min | 20 | 10% | 90% |
| 35 min | 20 | 10% | 90% |

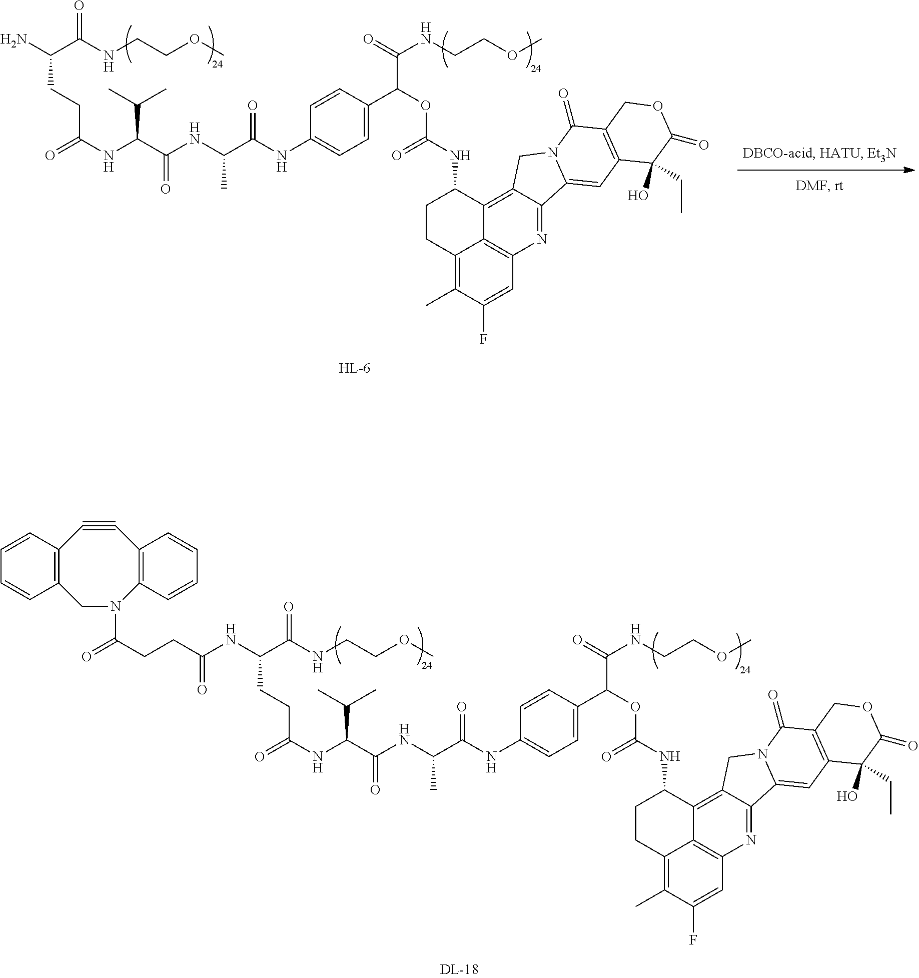

### 3-12. Preparation of 2SP-Glu (PEG₂₄)-VA-Hydra-PAB-Exatecan (Also Termed DL-19)

To a solution of HL-6 (25.7 mg, 0.0084 mmol), 6-(2-(Methylsulfonyl)pyrimidin-5-yl) hex-5-ynoic acid (2.3 mg, 0.0084 mmol) and HATU (4.8 mg, 0.0126 mmol) in anhydrous DMF (0.17 mL), Et₃N (3.5 μL, 0.0252 mmol) was added. The reaction mixture was stirred at room temperature for 1-2 hours. After the reaction was completed, the resulting mixture was purified with preparative HPLC to obtain 16.5 mg of DL-19 with 59.4% yield. HRMS (ESI) m/z found [(M+3H)/3]⁺, 1106.5778 C₁₅₅H₂₆₁FN₁₁O₆₂S³⁺, required 1106.5771

TABLE 18
HPLC condition (Column:
YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 80% | 20% |
| 5 min | 20 | 70% | 30% |
| 8 min | 20 | 64% | 36% |
| 30 min | 20 | 48% | 52% |
| 30.1 min | 20 | 10% | 90% |
| 34 min | 20 | 10% | 90% |
| 35 min | 20 | 10% | 90% |

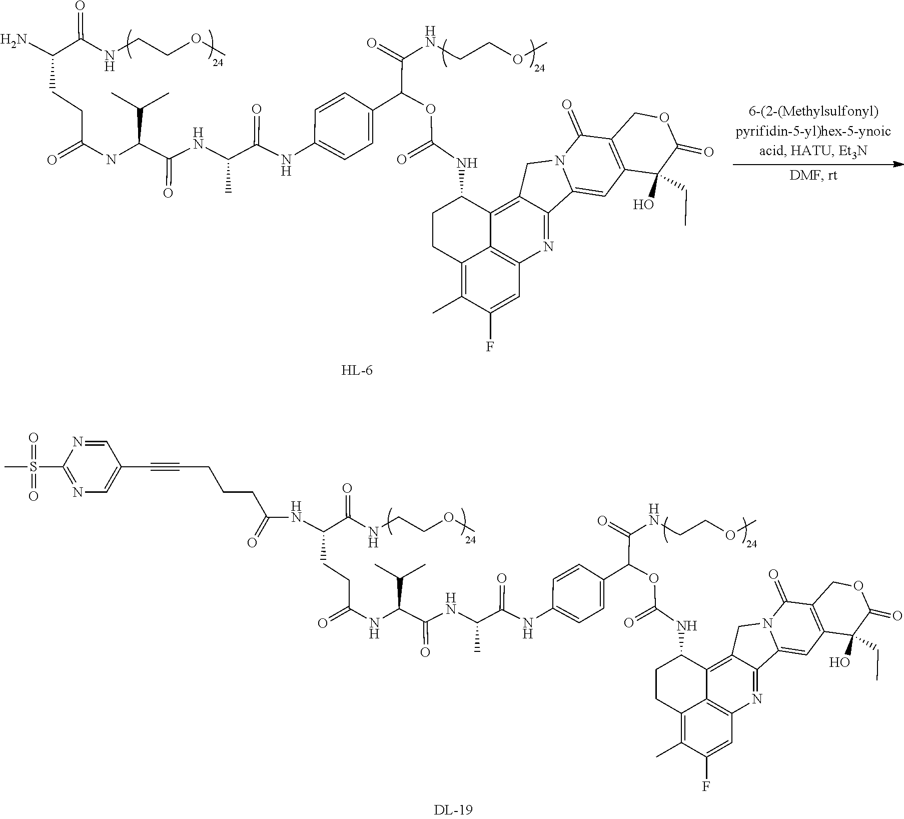

### 3-13. Preparation of MCCa-Glu (PEG₂₄)-VA-Hydra-PAB-Exatecan (Also Termed DL-20)

To a solution of HL-6 (19.9 mg, 0.0065 mmol) and SMCC (2.6 mg, 0.0078 mmol) in anhydrous DMF (0.13 mL), Et₃N (2.7 μL, 0.0195 mmol) was added. The reaction mixture was stirred at room temperature for 1-2 hours. After the reaction was completed, the resulting mixture was purified with preparative HPLC to obtain 10.9 mg of DL-20 with 51.4% yield. HRMS (ESI) m/z found [(M+3H)/3]⁺, 1096.2614 C₁₅₆H₂₆₄FN₁₀O₆₂³⁺, required 1096.2599.

TABLE 19
HPLC condition (Column:
YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 80% | 20% |
| 5 min | 20 | 70% | 30% |
| 8 min | 20 | 64% | 36% |
| 30 min | 20 | 48% | 52% |
| 30.1 min | 20 | 10% | 90% |
| 34 min | 20 | 10% | 90% |
| 35 min | 20 | 10% | 90% |

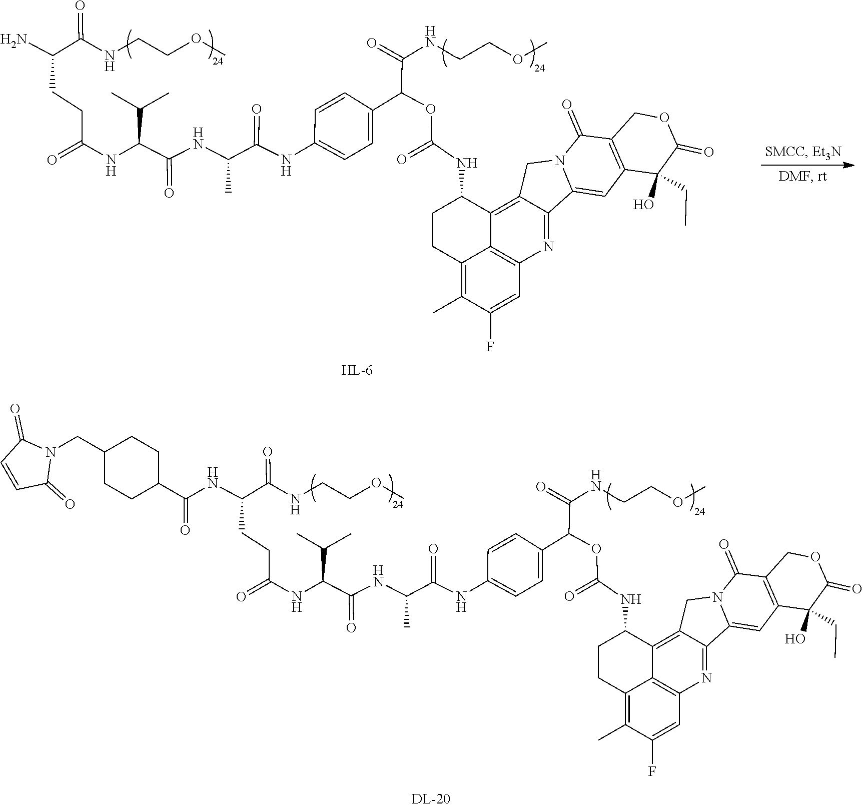

### 3-14. Preparation of BCN-GGVA-PAB (PSar20)-Exatecan (Also Termed DL-21)

N-PM-0025 (12.5 mg, 0.0115 mmol), polysarcosine (26.4 mg, 0.0172 mmol) and HATU (5.2 mg, 0.0138 mmol) were dissolved in anhydrous DMF (0.23 mL). NMM (3.8 μL, 0.0344 mmol) was added. The reaction mixture was stirred at 50° C. for 5-6 hours. After the reaction was completed, the resulting mixture was purified with preparative HPLC to obtain 10.4 mg of DL-21 with 34.8% yield. HRMS (ESI) m/z found [(M+3Na)/3]⁺, 878.0733 C₁₂₀H₁₇₀FN₂₉O₃₃Na₃³⁺, required 878.2725.

TABLE 20
HPLC condition (Column:
YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 75% | 25% |
| 6 min | 20 | 65% | 35% |
| 8 min | 20 | 65% | 35% |
| 10 min | 20 | 63% | 37% |
| 12 min | 20 | 63% | 37% |
| 14 min | 20 | 61% | 39% |
| 15 min | 20 | 50% | 50% |
| 15.1 min | 20 | 10% | 90% |
| 21 min | 20 | 10% | 90% |

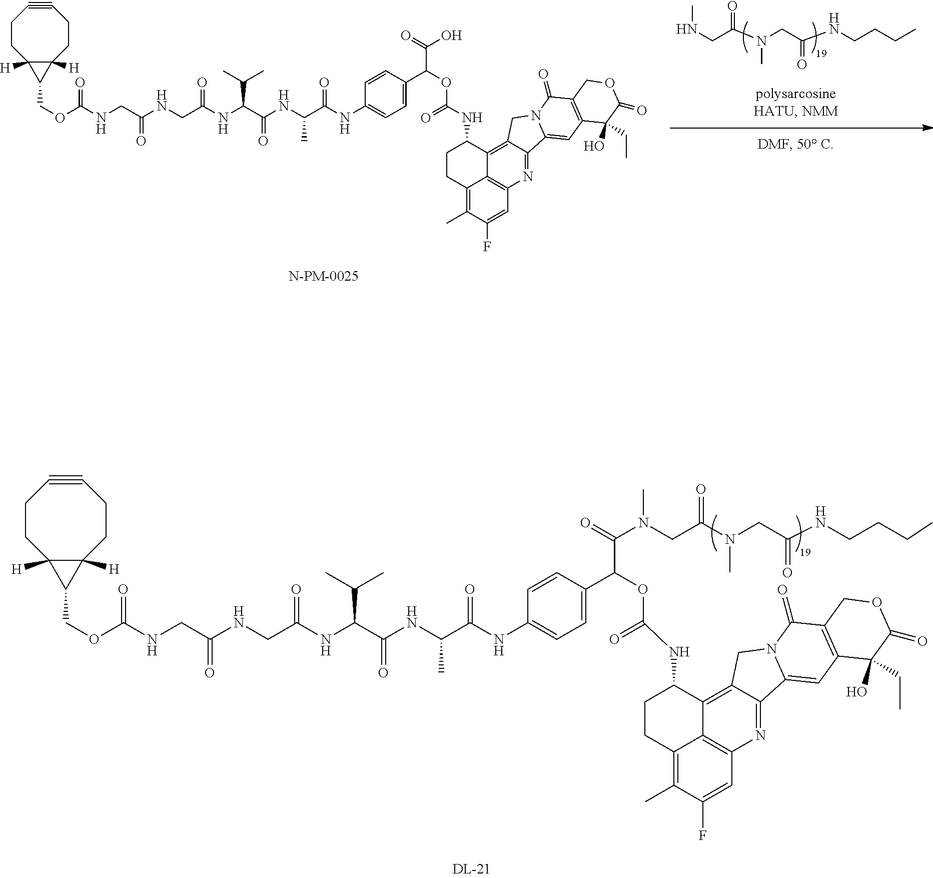

### 3-15. Preparation of BCN-Glu (PEG₂₄)-VA-PAB (PSar20)-Exatecan (Also Termed DL-22)

### Step 1:

Fmoc-VA-OH (703.5 mg, 1.713 mmol) and PAB (COOtBu)-OH (318.8 mg, 1.428 mmol) were dissolved in anhydrous CH₂Cl₂ (6.39 mL) and anhydrous MeOH (6.38 mL). EEDQ (529.7 mg, 2.142 mmol) was added. The resulting mixture was stirred at room temperature overnight. After the reaction was completed, the reaction mixture was concentrated in vacuo at 30-35° C. The crude product was purified by a flash silica gel column (EA/CH₂Cl₂=99/1 to 97/3, then CH₂Cl₂/MeOH=9/1) to obtain 687.6 mg of Fmoc-VA-PAB (COOtBu) with 78.2% yield.

Fmoc-VA-PAB (COOtBu) (674.5 mg, 1.095 mmol) and Bis(4-nitrophenyl) carbonate (866.4 mg, 2.848 mmol) were dissolved in DMF (3.3 mL). DIPEA (0.76 mL, 4.382 mmol) was added into the reaction solution. The reaction mixture was stirred at room temperature for 4-6 hours. After the reaction was completed, the reaction mixture was concentrated in vacuo at 30-35° C. The crude product was purified by a flash silica gel column (CH₂Cl₂/MeOH=15/1 to 9/1) to obtain 496.1 mg of Fmoc-VA-PAB (COOtBu)-PNP with 58.0% yield.

To a suspension of exatecan mesylate (295.0 mg, 0.555 mmol), Fmoc-VA-PAB (COOtBu)-PNP (481.5 mg, 0.617 mmol) in anhydrous DMF (6.1 mL), DIPEA (0.32 mL, 1.849 mmol) was added at room temperature. The suspension became a clear brown solution within five minutes. This mixture was stirred at room temperature overnight. After the reaction was completed, the reaction mixture was added dropwise into a stirring TBME (65.0 mL) to get precipitate. The solids were collected by filtration and followed with high vacuum drying to obtain crude Fmoc-VA-PAB (COOtBu)-Exatecan (398.5 mg, 60.0% yield). This crude product was used in the next step without further purification.

Fmoc-VA-PAB (COOtBu)-Exatecan (152.9 mg, 0.142 mmol) was dissolved in CH₂Cl₂/MeOH (3.4 mL, 1/1) and then cooled to 0° C. Et₂NH (0.85 mL) was added and the reaction mixture was stirred at 0-4° C. for 4-5 hours. After the reaction was completed, the reaction mixture was concentrated in vacuo at 30-35° C. to reduce the 2/3 solvent amount, and then added dropwise into a stirring TBME (30.0 mL) to get precipitate. The solids were collected by filtration and followed with high vacuum drying to obtain crude HL-7 (86.7 mg, 71.5% yield). This crude product was used in the next step without further purification.

### Step 2:

HL-7 (81.3 mg, 0.095 mmol), Fmoc-Glu (PEG₂₄) (145.1 mg, 0.100 mmol) and HATU (43.3 mg, 0.114 mmol) were dissolved in anhydrous DMF (0.95 mL). NMM (31.4 μL, 0.285 mmol) was added and the reaction mixture was stirred at room temperature for 1-2 hours. After the reaction was completed, the resulting mixture was purified with preparative HPLC to obtain 48.4 mg of Fmoc-Glu (PEG₂₄)-VA-PAB (COOtBu)-Exatecan with 22.4% yield. HRMS (ESI) m/z found [(M+H)]⁺, 2276.1406 C₁₁₄H₁₆₈FN₈O₃₈⁺, required 2276.1444.

TABLE 21
HPLC condition (Column:
YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 80% | 20% |
| 5 min | 20 | 70% | 30% |
| 8 min | 20 | 55% | 45% |
| 30 min | 20 | 40% | 60% |
| 30.1 min | 20 | 10% | 90% |
| 34 min | 20 | 10% | 90% |
| 35 min | 20 | 10% | 90% |

Fmoc-Glu (PEG₂₄)-VA-PAB (COOtBu)-Exatecan (42.6 mg, 0.018 mmol) was dissolved in CH₂Cl₂/MeOH (1.0 mL, 1/1) and then cooled to 0° C. Et₂NH (0.26 mL) was added and the reaction mixture was stirred at 0-4° C. for 4-5 hours. After the reaction was completed, the reaction mixture was concentrated in vacuo at 30-35° C. and azeotroped with toluene (5 mL×3) to remove excess Et₂NH. This crude product was then dissolved in anhydrous CH₂Cl₂ (0.71 mL) and cooled to 0° C. TFA (0.35 mL) was added dropwise. The reaction mixture was stirred at 0° C. overnight. After the reaction was completed, the reaction mixture was added dropwise into a stirring ether (11.0 mL) to get precipitate. The solids were collected by filtration and washed with ether (5 mL×3), and followed by dried with high vacuum to obtain crude HL-8. The crude product was purified with preparative HPLC to obtain 11.8 mg of HL-8 with 31.6% yield. HRMS (ESI) m/z found [(M+2H)/2]⁺, 999.5108 C₁₁₄H₁₆₈FN₈O₃₈⁺, required 999.5107.

TABLE 22
HPLC condition (Column:
YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 80% | 20% |
| 5 min | 20 | 70% | 30% |
| 8 min | 20 | 64% | 36% |
| 30 min | 20 | 48% | 52% |
| 30.1 min | 20 | 10% | 90% |
| 34 min | 20 | 10% | 90% |
| 35 min | 20 | 10% | 90% |

### Step 3:

HL-8 (10.1 mg, 0.0051 mmol) and BCN—OSu (2.2 mg, 0.0076 mmol) were dissolved in anhydrous DMF (0.10 mL). Et₃N (2.2 μL, 0.0153 mmol) was added. The resulting mixture was stirred at room temperature overnight. After the reaction was completed, the reaction mixture was concentrated in vacuo at 30-35° C. The crude intermediate was dissolved in anhydrous DMF 0.10 mL, followed by addition of PSar₂₀ (15.2 mg, 0.0102 mmol) and HATU (2.9 mg, 0.0076 mmol). NMM (1.7 μL, 0.0153 mmol) was added. The reaction mixture was stirred at room temperature for 4-6 hours. After the reaction was completed, the resulting mixture was purified with preparative HPLC to obtain 8.3 mg of DL-22 with 45.0% yield. HRMS (ESI) m/z found [(M+4H)/4]⁺, 913.2349 C₁₇₀H₂₇₄FN₂₉O₅₇⁴⁺, required 913.2354.

TABLE 23
HPLC condition (Column:
YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 80% | 20% |
| 5 min | 20 | 70% | 30% |
| 8 min | 20 | 64% | 36% |
| 30 min | 20 | 48% | 52% |
| 30.1 min | 20 | 10% | 90% |
| 34 min | 20 | 10% | 90% |
| 35 min | 20 | 10% | 90% |

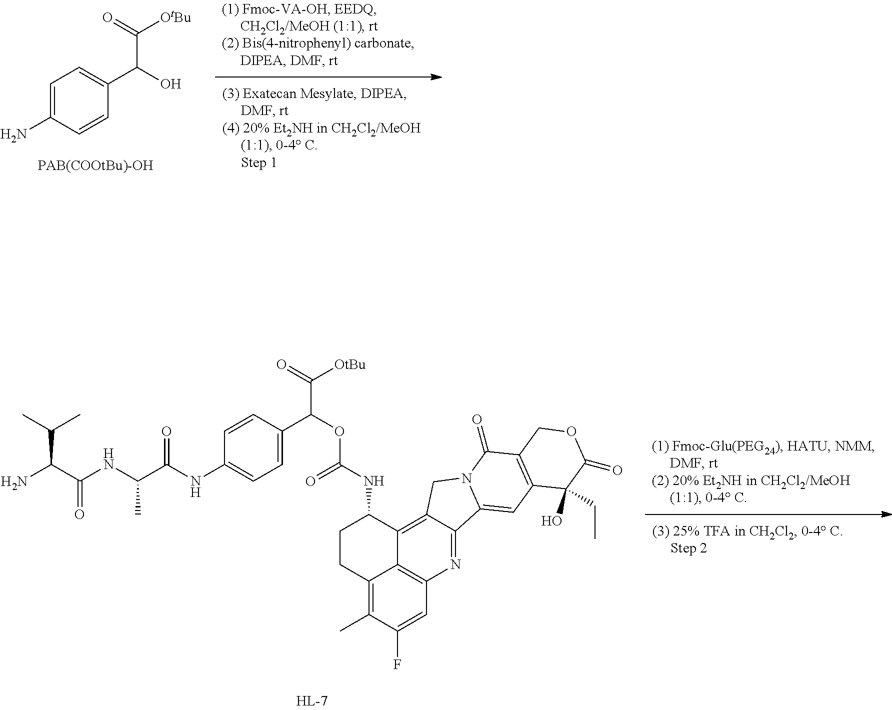 [SMILES: CC(C)(C)OC(=O)C(O)c1ccc(N)cc1 ; CC[C@@]1(O)C(=O)OCc2c1cc1n(c2=O)Cc2c-1nc1cc(F)c(C)c3c1c2[C@@H](NC(=O)OC(C(=O)OC(C)(C)C)c1ccc(NC(=O)[C@H](C)NC(=O)[C@@H](N)C(C)C)cc1)CC3]

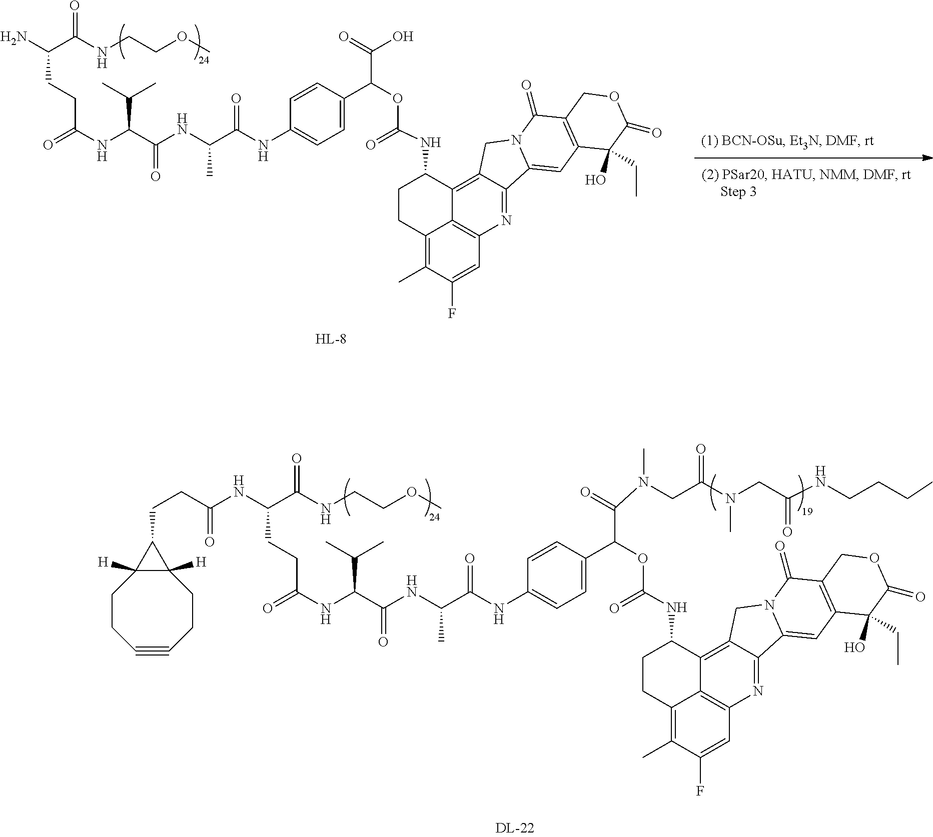

### 3-16. Preparation of BCN-Glu (PSar₂₀)-VA-PAB (PSar₂₀)-Exatecan (Also Termed DL-23)

### Step 1:

HL-7 (71.5 mg, 0.083 mmol), Fmoc-Glu (PSar₂₀) (162.1 mg, 0.088 mmol) and HATU (38.2 mg, 0.100 mmol) were dissolved in anhydrous DMF (1.6 mL). NMM (27.5 μL, 0.249 mmol) was added and the reaction mixture was stirred at room temperature for 1-2 hours. After the reaction was completed, the resulting mixture was purified with preparative HPLC to obtain 89.0 mg of Fmoc-Glu (PSar₂₀)-VA-PAB (COOtBu)-Exatecan with 39.7% yield. HRMS (ESI) m/z found [(M+2H)/2]⁺, 1341.6494 C₁₂₉H₁₇₉FN₂₈O₃₂²⁺, required 1341.6561.

TABLE 24
HPLC condition
(Column: YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 80% | 20% |
| 20 min | 20 | 40% | 60% |
| 23 min | 20 | 10% | 90% |
| 35 min | 20 | 10% | 90% |

Fmoc-Glu (PSar₂₀)-VA-PAB (COOtBu)-Exatecan (40.8 mg, 0.015 mmol) was dissolved in CH₂Cl₂/MeOH (0.99 mL, 1/1) and then cooled to 0° C. Et₂NH (0.24 mL) was added and the reaction mixture was stirred at 0-4° C. for 4-5 hours. After the reaction was completed, the reaction mixture was concentrated in vacuo at 30-35° C. and azeotroped with toluene (5 mL×3) to remove excess Et₂NH. This crude product was then dissolved in anhydrous CH₂Cl₂ (0.68 mL) and cooled to 0° C. TFA (0.33 mL) was added dropwise. The reaction mixture was stirred at 0° C. overnight. After the reaction was completed, the reaction mixture was added dropwise into a stirring ether (15.0 mL) to get precipitate. The solids were collected by filtration and washed with ether (5 mL×3), and followed by dried with high vacuum to obtain crude HL-9. The crude product was purified with preparative HPLC to obtain 10.8 mg of HL-9 with 29.6% yield. HRMS (ESI) m/z found [(M+2H)/2]⁺, 1202.5877 C₁₁₀H₁₆₁FN₂₈O₃₂²⁺, required 1202.5908.

TABLE 25
HPLC condition
(Column: YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 80% | 20% |
| 20 min | 20 | 40% | 60% |
| 23 min | 20 | 10% | 90% |
| 35 min | 20 | 10% | 90% |

### Step 2:

HL-9 (9.5 mg, 0.0040 mmol) and BCN—OSu (1.5 mg, 0.0051 mmol) were dissolved in anhydrous DMF (0.10 mL). Et₃N (1.7 μL, 0.0120 mmol) was added. The resulting mixture was stirred at room temperature overnight. After the reaction was completed, the reaction mixture was concentrated in vacuo at 30-35° C. The crude intermediate was dissolved in anhydrous DMF 0.10 mL, followed by addition of PSar₂₀ (12.0 mg, 0.0080 mmol) and HATU (1.8 mg, 0.0048 mmol). NMM (1.4 μL, 0.0120 mmol) was added. The reaction mixture was stirred at room temperature for 4-6 hours. After the reaction was completed, the resulting mixture was purified with preparative HPLC to obtain 3.9 mg of DL-23 with 24.9% yield.

TABLE 26
HPLC condition
(Column: YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 80% | 20% |
| 20 min | 20 | 40% | 60% |
| 23 min | 20 | 10% | 90% |
| 35 min | 20 | 10% | 90% |

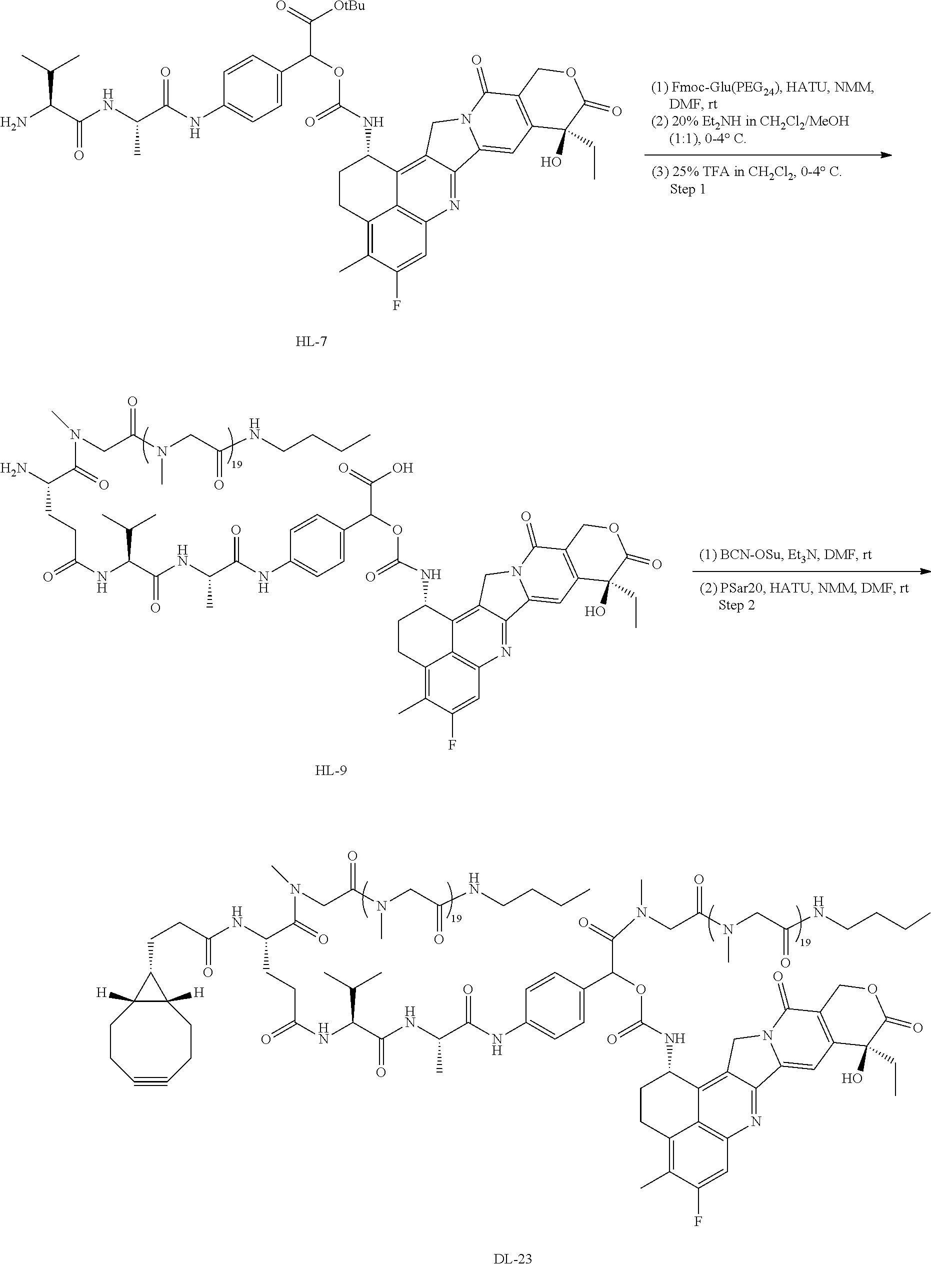

### 3-17. Preparation of BCN—VC-Hydra-PAB-Exatecan (Also Termed DL-24)

### Step 1:

Fmoc-VC—OH (489.3 mg, 0.985 mmol) and PAB (COOtBu)-OH (200.0 mg, 0.895 mmol) were dissolved in anhydrous CH₂Cl₂ (2.0 mL) and anhydrous MeOH (2.0 mL). EEDQ (310.1 mg, 1.254 mmol) was added. The resulting mixture was stirred at room temperature overnight. After the reaction was completed, the reaction mixture was added dropwise into a stirring ethyl ether (50.0 mL) to get precipitate. The solids were collected by filtration and followed with high vacuum drying to obtain crude Fmoc-VC-PAB (COOtBu)-OH (445.1 mg, 71.0% yield). This crude product was used in the next step without further purification.

Fmoc-VC-PAB (COOtBu) (445.0 mg, 0.634 mmol) and Bis(4-nitrophenyl) carbonate (578.7 mg, 1.902 mmol) were dissolved in DMF (4.5 mL). DIPEA (0.33 mL, 1.902 mmol) was added into the reaction solution. The reaction mixture was stirred at room temperature overnight. After the reaction was completed, the reaction mixture was added dropwise into a stirring ethyl ether (50.0 mL) to get precipitate. The solids were collected by filtration and followed with high vacuum drying to obtain crude Fmoc-VC-PAB (COOtBu)-PNP (300.0 mg, 81.0% yield). This crude product was used in the next step without further purification.

To a suspension of exatecan mesylate (183.9 mg, 0.346 mmol) and Fmoc-VC-PAB (COOtBu)-PNP (300.0 mg, 0.346 mmol) in anhydrous DMF (1.73 mL), DIPEA (0.18 mL, 1.038 mmol) was added at room temperature. The suspension became a clear brown solution within 5 minutes. This mixture was stirred at room temperature overnight. After the reaction was completed, the reaction mixture was added dropwise into a stirring ethyl ether (20.0 mL) to get precipitate. The solids were collected by filtration and followed with vacuum drying to obtain a crude product. The crude product was purified by a flash silica gel column (CH₂Cl₂/MeOH=12/1 to 7/1) to obtain 250.0 mg of HL-10 with 62.0% yield.

### Step 2:

A stirring suspension of HL-10 (100.0 mg, 0.115 mmol) in DCM (4.0 mL) was cooled to 0° C. TFA (1.98 mL) was added at 0° C. slowly. The reaction mixture was stirred at 0-4° C. overnight. After the reaction was completed, the reaction mixture was added dropwise into a stirring ethyl ether (60 mL) to get precipitate. The solids were collected by filtration and followed with high vacuum drying to obtain crude Fmoc-VC-PAB (COOH)-Exatecan (64.5 mg, 68% yield). This crude product was used in the next step without further purification.

Fmoc-VC-PAB (COOH)-Exatecan (64.5 mg, 0.058 mmol), NH₂-PEG₂₄-OMe (75.7 mg, 0.070 mmol) and HATU (33.1 mg, 0.087 mmol) were dissolved in anhydrous DMF (0.65 mL). NMM (12.8 μL, 0.116 mmol) was added and the reaction mixture was stirred at room temperature for 1-2 hours. After the reaction was completed, the reaction mixture was added dropwise into a stirring ethyl ether (10 mL) to get precipitate. The solids were collected by filtration and followed with vacuum drying to obtain a crude product. The crude product was purified with preparative HPLC to obtain 47.8 mg of Fmoc-VC-Hydra-PAB-Exatecan with 37.0% yield.

TABLE 27
HPLC condition
(Column: YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 90% | 10% |
| 5 min | 20 | 50% | 50% |
| 20 min | 20 | 45% | 55% |
| 21 min | 20 | 10% | 90% |
| 25 min | 20 | 10% | 90% |

Fmoc-VC-Hydra-PAB-Exatecan (47.8 mg, 0.022 mmol) was dissolved in CH₂Cl₂/MeOH (1.4 mL, 1/1). Et₂NH (0.16 mL) was added and the reaction mixture was stirred at room temperature overnight. After the reaction was completed, the reaction mixture was added dropwise into a stirring ethyl ether (15 mL) to get precipitate. The solids were collected by filtration and followed with high vacuum drying to obtain crude NH₂—VC-Hydra-PAB-Exatecan. This crude product was used in the next step without further purification. The crude product and BCN—OSu (6.4 mg, 0.022 mmol) were dissolved in anhydrous DMF (0.4 mL). DIPEA (11.5 μL, 0.066 mmol) was added. The resulting mixture was stirred at room temperature for 0.5-1 hour. After the reaction was completed, the resulting mixture was purified with preparative HPLC to obtain 21.3 mg of DL-24 with 40.0% yield. HRMS (ESI) m/z found [(M+2H)/2]⁺, 1066.0572 C₁₀₄H₁₆₂FN₉O₃₆²⁺, required 1066.0553.

TABLE 28
HPLC condition
(Column: YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 90% | 10% |
| 5 min | 20 | 55% | 45% |
| 20 min | 20 | 50% | 50% |
| 21 min | 20 | 10% | 90% |
| 25 min | 20 | 10% | 90% |

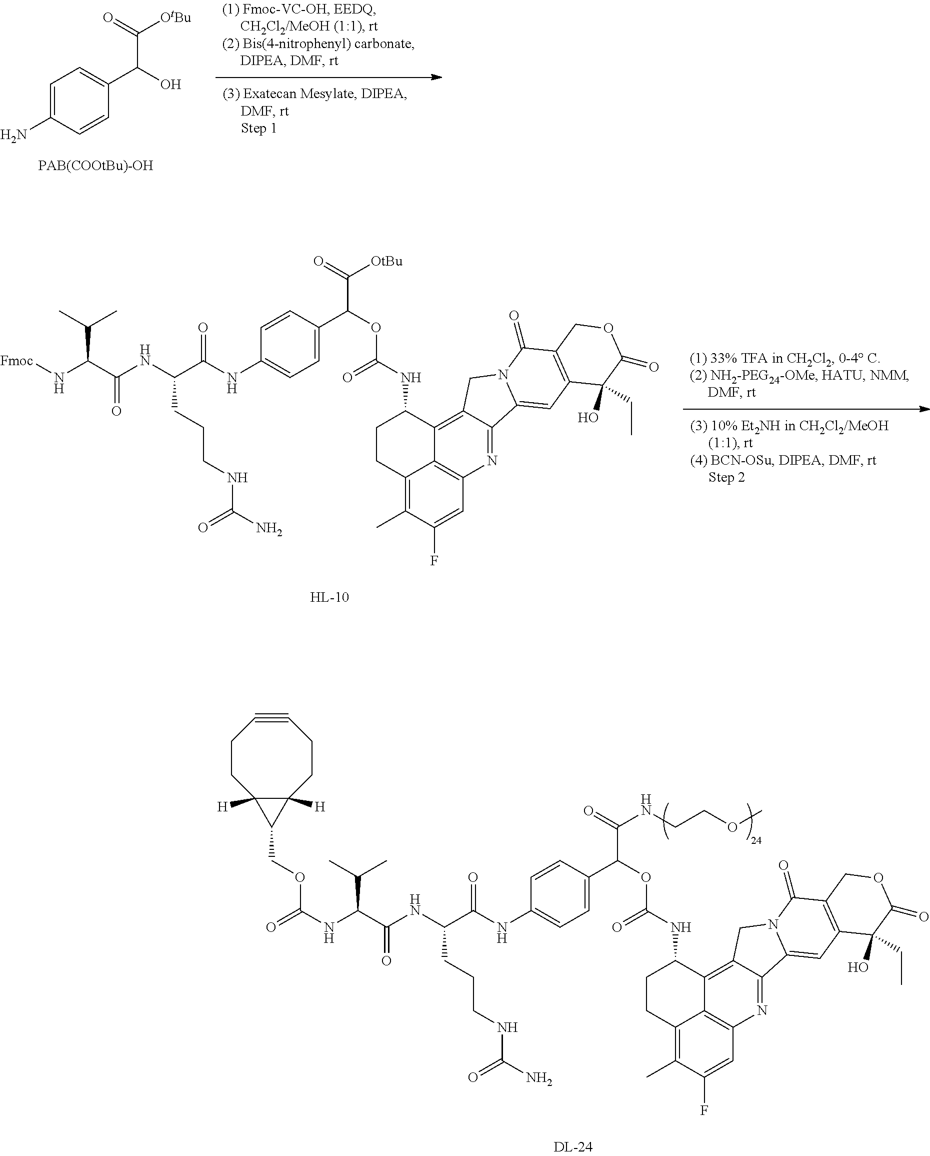

### 3-18. Preparation of BCN-GGVC-Hydra-PAB-Exatecan (Also Termed DL-25)

HL-10 (150.0 mg, 0.129 mmol) was dissolved in CH₂Cl₂/MeOH (4.0 mL, 1/1) and the reaction solution was cooled to 0-4° C. Et₂NH (0.45 mL) was added and the reaction mixture was stirred at 0-4° C. for three days. After the reaction was completed, the reaction mixture was added dropwise into a stirring ethyl ether (45 mL) to get precipitate. The solids were collected by filtration and followed with high vacuum drying to obtain crude NH₂—VC-PAB (COOtBu)-Exatecan (82.5 mg) without purification. The crude NH₂—VC-PAB (COOtBu)-Exatecan (82.5 mg, 0.088 mmol), Boc-GG-OH (20.4 mg, 0.088 mmol) and HATU (40.1 mg, 0.106 mmol) were dissolved in anhydrous DMF (1.76 mL). NMM (29.1 μL, 0.264 mmol) was added. The resulting mixture was stirred at room temperature overnight. After the reaction was completed, the resulting mixture was purified with preparative HPLC to obtain 66.7 mg of Boc-GGVC-PAB (COOtBu)-Exatecan with 65.9% yield.

TABLE 29
HPLC condition
(Column: YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 90% | 10% |
| 5 min | 20 | 50% | 50% |
| 20 min | 20 | 45% | 55% |
| 21 min | 20 | 10% | 90% |
| 25 min | 20 | 10% | 90% |

A stirring suspension of Boc-GGVC-PAB (COOtBu)-Exatecan (65.0 mg, 0.056 mmol) in CH₂Cl₂ (1.1 mL) was cooled to 0° C. TFA (0.54 mL) was added at 0° C. slowly. The reaction mixture was stirred at 0-4° C. overnight. After the reaction was completed, the reaction mixture was added dropwise into a stirring ethyl ether (20 mL) to get precipitate. The solids were collected by filtration and followed with high vacuum drying to obtain crude NH₂-GGVC-PAB (COOH)-Exatecan without further purification. The crude NH₂-GGVC-PAB (COOH)-Exatecan and BCN—OSu (16.3 mg, 0.056 mmol) were dissolved in anhydrous DMF (1.1 mL). DIPEA (29.3 μL, 0.168 mmol) was added. The resulting mixture was stirred at room temperature for 0.5-1 hour. After the reaction was completed, the reaction mixture was added dropwise into a stirring ethyl ether (45 mL) to get precipitate. The solids were collected by filtration and followed with high vacuum drying to obtain crude BCN-GGVC-PAB (COOH)-Exatecan (18.4 mg) without purification. Crude BCN-GGVC-PAB (COOH)-Exatecan (18.4 mg, 0.016 mmol), NH₂-PEG₂₄-OMe (34.8 mg, 0.032 mmol) and HATU (7.3 mg, 0.019 mmol) were dissolved in anhydrous DMF (0.32 mL). NMM (5.3 μL, 0.048 mmol) was added and the reaction mixture was stirred at room temperature overnight. After the reaction was completed, the reaction mixture was purified with preparative HPLC to obtain 17.8 mg of DL-25 with 50.6% yield. HRMS (ESI) m/z found [(M+3H)/3]⁺, 749.0508 C₁₀₈H₁₆₉FN₁₁O₃₈³⁺, required 749.0459.

TABLE 30
HPLC condition
(Column: YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 90% | 10% |
| 5 min | 20 | 60% | 40% |
| 20 min | 20 | 56% | 44% |
| 21 min | 20 | 10% | 90% |
| 25 min | 20 | 10% | 90% |

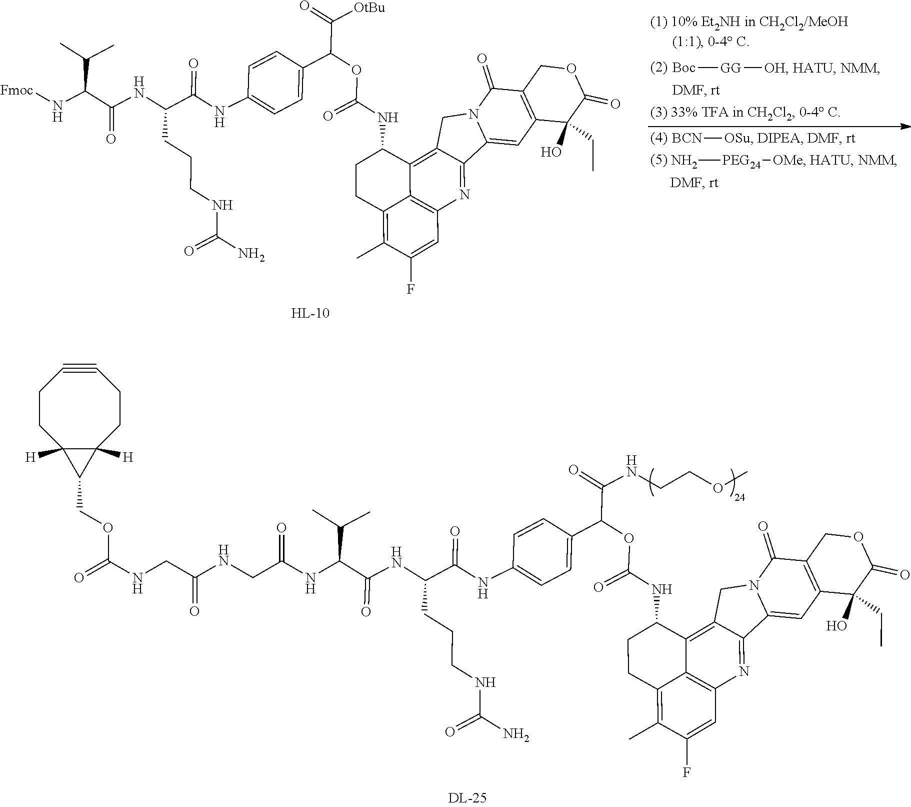

### 3-19. Preparation of BCN-GGFG-Hydra-PAB-Exatecan (Also Termed DL-26)

### Step 1:

Boc-GGFG-OH (498.0 mg, 1.141 mmol) and PAB (COOtBu)-OH (212.3 mg, 0.951 mmol) were dissolved in anhydrous CH₂Cl₂ (4.2 mL) and anhydrous MeOH (4.2 mL). EEDQ (352.7 mg, 1.426 mmol) was added and the resulting mixture was stirred at room temperature overnight. After the reaction was completed, the reaction mixture was concentrated in vacuo at 30-35° C. The crude product was purified by a flash silica gel column (CH₂Cl₂/MeOH=12/1 to 9/1) to obtain 603.5 mg of Boc-GGFG-PAB (COOtBu) with 98.9% yield.

Boc-GGFG-PAB (COOtBu) (603.5 mg, 0.940 mmol) and Bis(4-nitrophenyl) carbonate (1.43 g, 4.703 mmol) were dissolved in anhydrous CH₂Cl₂/DMF (9.4 mL, 4/1). DIPEA (0.82 mL, 4.703 mmol) was added into the reaction solution. The reaction mixture was stirred at room temperature for 5-6 hours. After the reaction was completed, the reaction mixture was concentrated in vacuo at 30-35° C. The crude product was purified by a flash silica gel column (CH₂Cl₂/EA=3/1 to CH₂Cl₂/MeOH=15/1) to obtain 350.5 mg of Boc-GGFG-PAB (COOtBu)-PNP with 46.2% yield.

To a suspension of exatecan mesylate (254.0 mg, 0.478 mmol) and Boc-GGFG-PAB (COOtBu)-PNP (350.5 mg, 0.434 mmol) in anhydrous CH₂Cl₂/DMF (4.3 mL, 1/1), DIPEA (227.0 μL, 1.303 mmol) was added. The reaction mixture was stirred at room temperature overnight. After the reaction was completed, the reaction mixture was concentrated in vacuo at 30-35° C. The crude product was purified by a flash silica gel column (CH₂Cl₂/EA=1/1 to CH₂Cl₂/MeOH=15/1) to obtain 221.7 mg of HL-11 with 46.3% yield.

### Step 2:

A stirring suspension of HL-11 (100.0 mg, 0.091 mmol) in CH₂Cl₂ (1.7 mL) was cooled to 0° C. TFA (0.83 mL) was added at 0° C. slowly. The reaction mixture was stirred at 0-4° C. overnight. After the reaction was completed, the reaction mixture was added dropwise into a stirring ethyl ether (25 mL) to get precipitate. The solids were collected by filtration and followed with high vacuum drying to obtain crude NH₂-GGFG-PAB (COOH)-Exatecan without further purification. The crude NH₂-GGFG-PAB (COOH)-Exatecan and BCN—OSu (26.5 mg, 0.091 mmol) were dissolved in anhydrous DMF (1.8 mL). DIPEA (47.6 μL, 0.273 mmol) was added. The resulting mixture was stirred at room temperature for 0.5-1 hour. After the reaction was completed, the reaction mixture was added dropwise into a stirring ethyl ether (30 mL) to get precipitate. The solids were collected by filtration and followed with high vacuum drying to obtain crude BCN-GGFG-PAB (COOH)-Exatecan (65.1 mg) without purification. Crude BCN-GGFG-PAB (COOH)-Exatecan (65.1 mg, 0.058 mmol), NH₂-PEG₂₄-OMe (126.1 mg, 0.116 mmol) and HATU (26.4 mg, 0.069 mmol) were dissolved in anhydrous DMF (1.2 mL). NMM (19.2 μL, 0.174 mmol) was added and the reaction mixture was stirred at room temperature overnight. After the reaction was completed, the reaction mixture was purified with preparative HPLC to obtain 52.8 mg of DL-26 with 41.6% yield. HRMS (ESI) m/z found [(M+2H)/2]⁺, 1097.0405 C₁₀₈H₁₈₀FN₉O₃₇²⁺, required 1097.0449.

TABLE 31
HPLC condition
(Column: YMC-Actus Trait C18 250 × 20 mm, 5 μm, 12 nm)

| Gradient: | | | |
|---|---|---|---|
| Time | Flow (ml/min) | H₂O | ACN |
| 0 min | 10 | 90% | 10% |
| 1 min | 20 | 70% | 30% |
| 6 min | 20 | 45% | 55% |
| 12 min | 20 | 37% | 63% |
| 13 min | 20 | 35% | 65% |
| 14 min | 20 | 32% | 68% |
| 16 min | 20 | 30% | 70% |
| 17 min | 20 | 27% | 73% |
| 19 min | 20 | 27% | 73% |
| 20 min | 20 | 10% | 90% |
| 24 min | 20 | 10% | 90% |

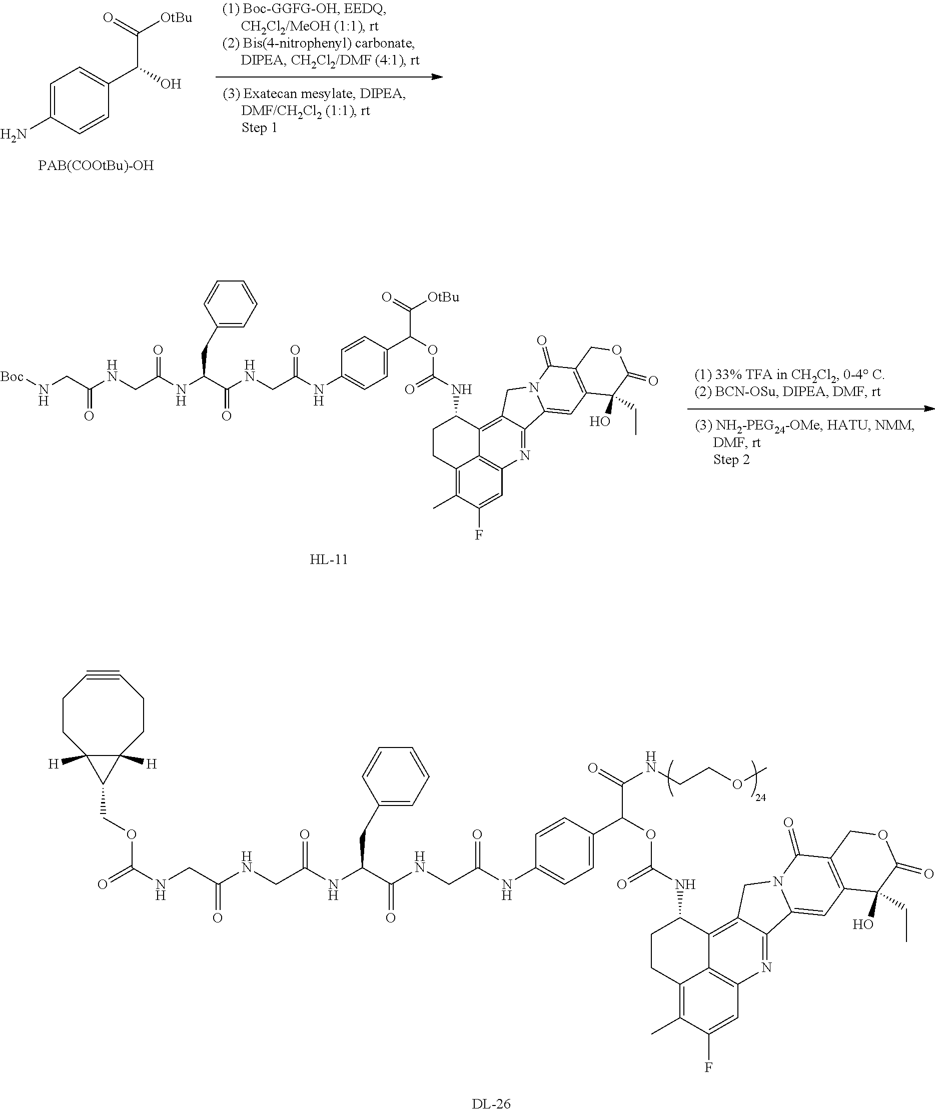

### Example 4. ADC Preparation (ADC-1 to ADC-4)

### 4-1. ADC Preparation by Using MCCA-PEG₂₄-VA-PAB-Exatecan (DL-1)

R4702 and TX05 mAbs (10 mg/mL, total 50 mL) dissolved in reaction buffer (50 mM Histidine, 20 mM EDTA, pH 7.0) was cooled to 12-16° C. R4702 and TX05 were treated with TCEP-HCl (2.29 mg; 0.00799 mmol) in the reaction buffer (0.46 mL) for 2-6 hours at 12-16° C. In order to reduce the antibody solution, the linker-payload DL-1/N-PM-0017 (39.94 mg; 0.0183 mmol) dissolved in DMSO was added to allow antibody conjugation for one hour at 12-16° C. After the conjugation was completed, the buffer was changed to a storage buffer (20 mM sodium acetate, pH 5.0 with 0.1% (w/w) polysorbate 80) via UF/DF dialysis membrane to achieve a final concentration of 10.14 mg/mL and a total volume of 41.9 mL. The average drug-to-antibody ratio (DAR) values for R4702-MCCA-PEG₂₄-ADC (ADC-1) and TX05-MCCA-PEG₂₄-ADC (ADC-3) were 4.5 and 4.7, respectively, determined by hydrophilic interaction chromatography (HIC).

### 4-2. ADC Preparation by Using DBCO-PEG₂₄-VA-PAB-Exatecan (DL-2)

DL-2 (16.35 mg) was dissolved in 1635 μL DMSO to form a DL-2 solution. The DL-2 solution (1614 μL) was added slowly to R4702-(NSCT-diN3)₂ solution (18 mL, antibody at a concentration of 5 mg/mL in 20 mM NaOAc, pH 5.0) and shaken at 25° C. for 6.5 hours. After the conjugation was completed, the residual DL-2 was partially removed by buffer exchange (20 mM NaOAc, pH 5.0) using PES Amicons. The crude ADC was further purified by HIC column to afford ADC-2. After exchanging the buffer to a storage buffer (20 mM NaOAc, pH 5.0), R4702-DBCO-PEG₂₄-ADC (ADC-2) was adjusted to 5.02 mg/mL and sterilized by passing through ProMax™ Syringe Filter (PVDF, 0.22 μm). Finally, 9.9 mL of ADC-2 was produced with a DAR value of 3.8 (determined by HIC).

Furthermore, DL-2 (34.58 mg) was dissolved in 1729 μL DMSO to form a DL-2 solution. The DL-2 solution (1108 μL) was added slowly to TX05-(NSCT-di-N3)₂ solution (25.33 mL, antibody at a concentration of 4.88 mg/mL in 20 mM NaOAc, pH 5.0) and stirred at 25° C. for 6 hours. After the conjugation was completed, the crude ADC was further purified by using Spectrum® Hollow Fiber Filter Modules (buffer: 20 mM NaOAc, pH 5.0) to afford TX05-DBCO-PEG₂₄-ADC (ADC-4). ADC-4 was adjusted to around 5 mg/mL and sterilized by passing through ProMax™ Syringe Filter (PVDF, 0.22 μm). Finally, 17.7 mL of ADC-4 (concentration: 4.53 mg/mL) was produced with a DAR value of 3.9 (determined by HIC).

### 4-3. ADC Preparation by Using MCCA-Linear PEG₁₂-VA-PAB-Exatecan (DL-3)

Anti-TROP2 antibody R4702 (10.0 mg/mL, 7.0 mL) in a reaction buffer (50 mM Histidine, 20 mM EDTA, pH 7.0) was cooled to 12-16° C. This R4702 solution was treated with TCEP-HCl (0.32 mg, 0.00112 mmol) in the reaction buffer (0.032 mL) for 3 hours at 12-16° C. To reduce the antibody solution, DL-3 (4.9 mg, 0.00327 mmol) in DMSO (0.25 mL) was added to allow conjugation for 1 hour at 12-16° C. After the conjugation was completed, the buffer of ADC was changed to a storage buffer (20 mM sodium acetate, pH 5.0) via centrifugation with Amicon to afford the ADC (10.10 mg/ml, 6.0 mL). The DAR value of the ADC was 3.7, determined by reverse-phase liquid chromatography (RPLC).

### Example 5. ADC Analysis (ADC-1 to ADC-4)

### 5-1. CE-SDS Analysis of Antibody Deglycosylation and Transglycosylation

The CE-SDS analysis was conducted under reducing conditions. Beckman Coulter PA800Plus system equipped with a UV photodiode array detector (220 nm wavelength employed) was used in this test. A bare fused-silica capillary (50 m ID×30 cm total length) with the 20 cm effective capillary separation length was rinsed with 0.1 M NaOH, 0.1 M HCl and SDS gel buffer prior to injection. Electrokinetic injection mode was applied at −5 kV for 20 seconds in reverse polarity and followed by applying a −15 kV voltage for capillary separation. The total separation time was 35 minutes. 60 μg of test article was sampled and diluted in 120 μL sample buffer, which was 1% SDS in diluted PBS, pH 7.0. The diluted sample was then mixed with 5 μL of 2-ME and 2 μL of 10 kDa internal standard, followed by incubation at 65° C. for 10 minutes. Finally, the sample was cooled down at room temperature for CE-SDS analysis.

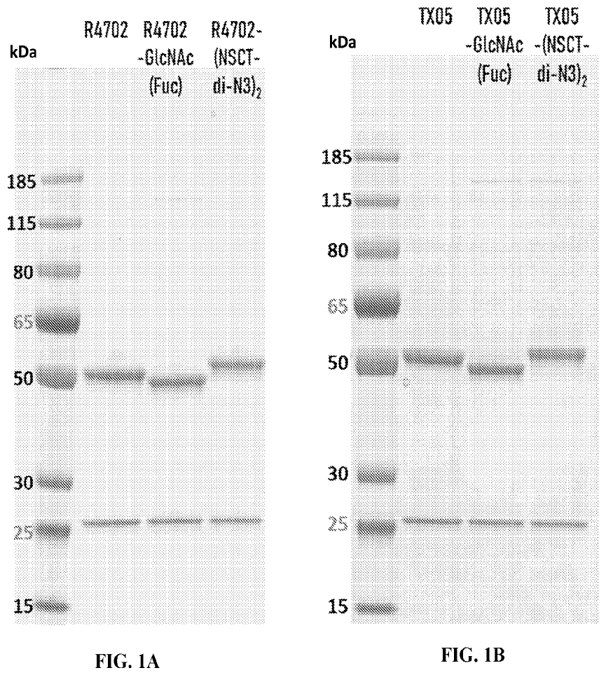

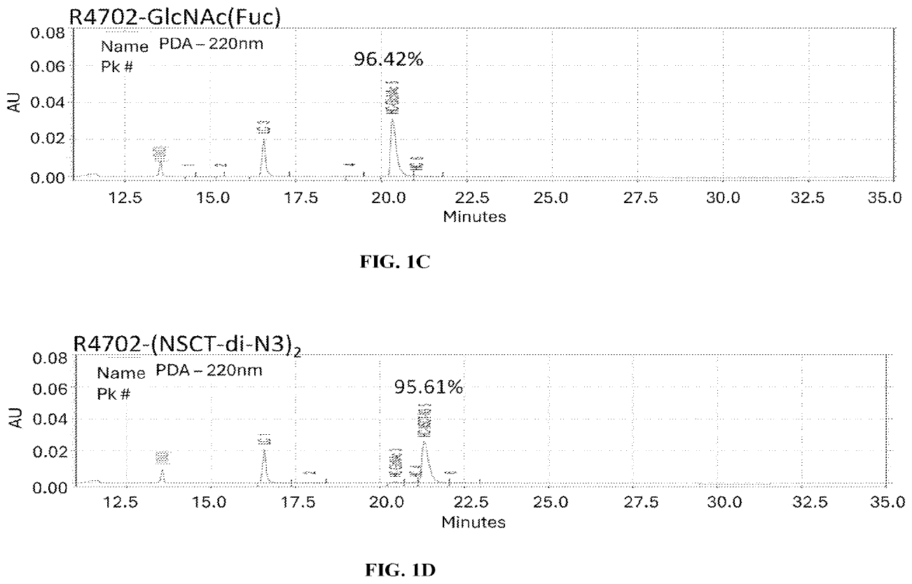

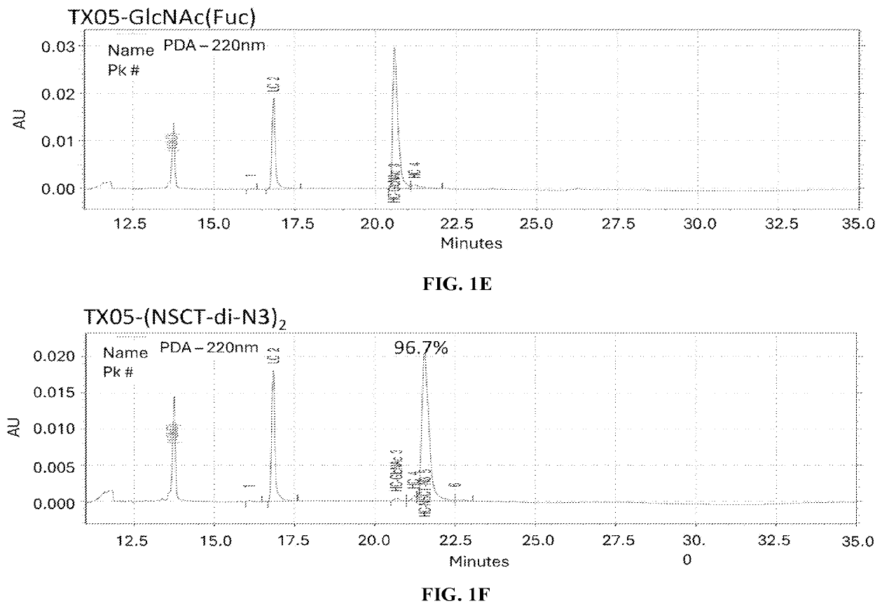

EndoSz-D234M showed high deglycosylation and transglycosylation activity. R4702 and TX05 mAbs pretreated with EndoSz-D234M were used to produce R4702-DBCO-PEG₂₄-ADC (ADC-2) and TX05-DBCO-PEG₂₄-ADC (ADC-4) as an exemplary glycan-engineered ADC. For ADC-2, R4702 mAb were deglycosylated by EndoSz-D234M together with additional enzyme EndoH to cleave high mannose glycans. The yield of deglycosylated R4702 with one GlcNAc or potential fucose (termed R4702-GlcNAc (Fuc)) was about 96.42%. Next, R4702-GlcNAc (Fuc) was mixed with 20 equivalents of a modified complex type N-glycan (NSCT-2) at 37° C. for 1.5 hour to generate R4702-(NSCT-di-N3)₂. The CE-SDS results indicated that 96.51% R4702-(NSCT-di-N3)₂ was produced by EndoSz-D234M. For ADC-4, EndoSz-D234M was used to hydrolyze the biantennary hybrid glycan on TX05 mAb to generate 94.5% TX05-GlcNAc (Fuc). In the transglycosylation step, NSCT-2 was added to a mixture of TX05-GlcNAc (Fuc) and EndoSz-D234M. With 15 equivalents of NSCT-2 to TX05-GlcNAc (Fuc), 96.7% of TX05-(NSCT-di-N3)₂ was obtained by EndoSz-D234M catalysis at 15° C. for 4.5 hours of incubation (FIGS. 1C-1F). These data demonstrate that EndoSz-D234M facilitates the generation of mAb-(NSCT-di-N3)₂, which is an exemplary starting material to produce glycan-engineered ADCs.

### 5-2. Intact Molecular Weight (MW) Analysis by LC-MS

Intact MW analysis was conducted by Q-Exactive mass spectrometer (Thermo Scientific) coupled with vanquish HPLC system (Thermo). The LC separation was performed using Agilent PLRP-S column with the gradient program. Test sample was diluted to 0.5 mg/mL with H₂O and no further deglycosylation or reduction occurred before LC-MS analysis. Full MS scans were performed within the range of m/z 1500-5000 for molecular weight analyses. Protein Deconvolution 4.0 was used to process the raw data and obtain the molecular weight.

The results of intact MW analysis and reduced MS analysis for each species are shown in Table 32. It demonstrates the deglycosylation and transglycosylation by EnddoSz-D234M. The mass difference between R4702-GlcNAc (Fuc) and R4702 indicated that the N-Glycan structure on R4702-GlcNAc (Fuc) was GlcNAc (Fuc). The N-glycan of R4702 could be cleaved to form R4702-GlcNAc (Fuc), which was evaluated by the intact MW analysis. The MW of R4702-(NSCT-di-N3)₂ illustrated NSCT-2 was successfully transferred to R4702-GlcNAc (Fuc) by EndoSz-D234M. The MW of R4702-DBCO-PEG₂₄-ADC (ADC-2) demonstrated there were four payloads conjugated with R4702. Furthermore, the results of reduced-MW analysis also demonstrated that the modification was located on heavy chain.

TABLE 32
The result of intact MW analysis and reduced MS analysis

| | | ΔMW to Mab/ | Measured | Measured |
|---|---|---|---|---|
| | Measured | Possible Structure of | Heavy | Light Chain |
| Sample | Intact MW | Modifier | Chain MW | MW |
| De-glycosylated | 144242.0 | — | 48972.5 | 23342.0 |
| R4702 by PNGase | | | | |
| (Mab) | | | | |
| R4702-GlcNAc(Fuc) | 144935.6 | Δ693.6/ | 49140.6 | 23341.1 |
| | | Two of GlcNAc-Fuc | | |
| R4702-(NSCT-di-N3)₂ | 149745.3 | Δ5503.3/ | 51517.7 | 23341.1 |
| | | Two of NSCT-di-N3 | | |
| ADC-2 | 158708.4 | Δ14466.4/ | 56027.0 | 23341.1 |
| | | Two of (NSCT-di- | | |
| | | (linker payload)) | | |

### 5-3. DAR Analysis in the Presence of Human Serum Albumin

Maleimide linkers have been used extensively for conjugation of antibody and payloads. Nevertheless, the thioether connection undergoes deconjugation via a retro-Michael reaction, resulting in payload loss and lower the therapeutic efficacy of ADCs. Moreover, the maleimide-linked payload can attach to plasma thiols (such as human serum albumin, HSA), causing off target toxicity. To examine the DAR value change in the presence of HSA, R4702-MCCA-PEG₂₄-ADC (ADC-1) and R4702-DBCO-PEG₂₄-ADC (ADC-2) were added to 3% HSA in PBS to a final concentration of 500 g/mL and incubated at 37° C. for 0, 24, or 144 hours in a shaking water bath. The incubated samples were purified with anti-idiotype antibody-coated streptavidin magnetic beads. The mixture was gently shaken for 1.5 hour at room temperature then washed three times with HEPES buffered salt solution, two times with deionized water and eluted with 2% formic acid. For DAR change calculation, the eluted ADCs were reduced with TCEP (final concentration, 20 mM) at room temperature for 30 minutes to form light chain (LC) and heavy chain (HC) fragments and followed by LC-HRMS analysis. The following formula was used for average DAR calculation:

$$( LC 1 / ( LC 0 + LC 1 ) ) \times 2 + ( HC 1 / ( HC 0 + HC 1 + HC 2 + HC 3 ) ) \times 2 + ( HC 2 / ( HC 0 + HC 1 + HC 2 + HC 3 ) ) \times 4 + HC 3 / ( HC 0 + HC 1 + HC 2 + HC 3 ) ) \times 6 .$$

where LC0 and LC1 refer to the reconstruction area of the signal for the light chain with zero or one payload, respectively, and HC0, HC1, HC2, and HC3 refer to the reconstruction area of the signal for the heavy chain with 0, 1, 2, or 3 payloads, respectively.

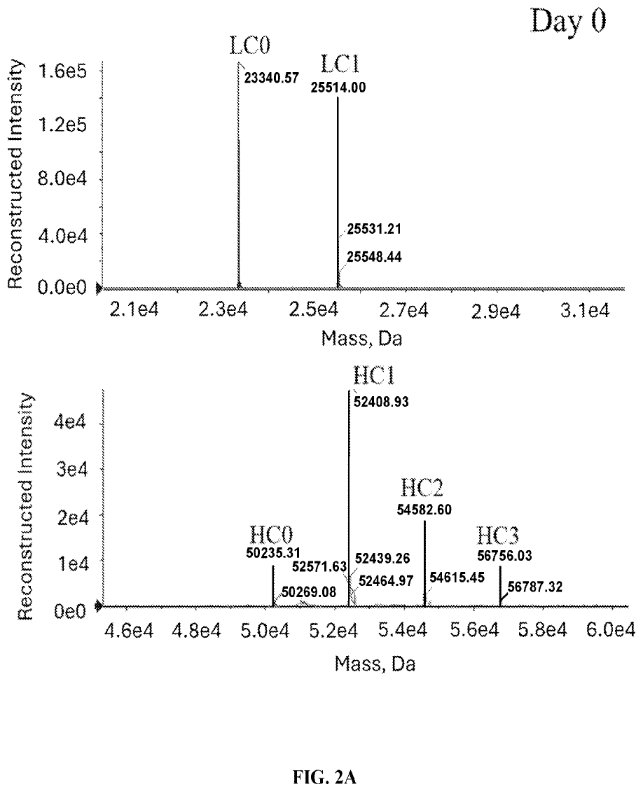

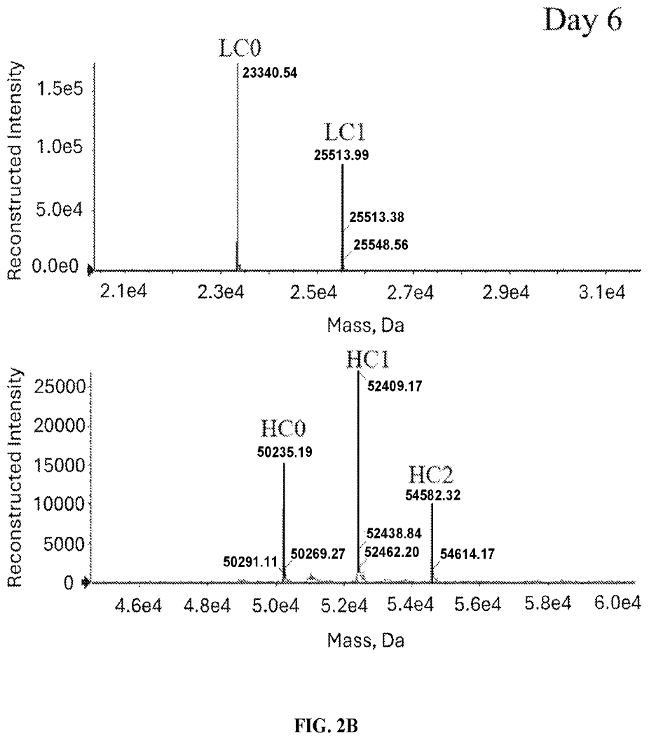

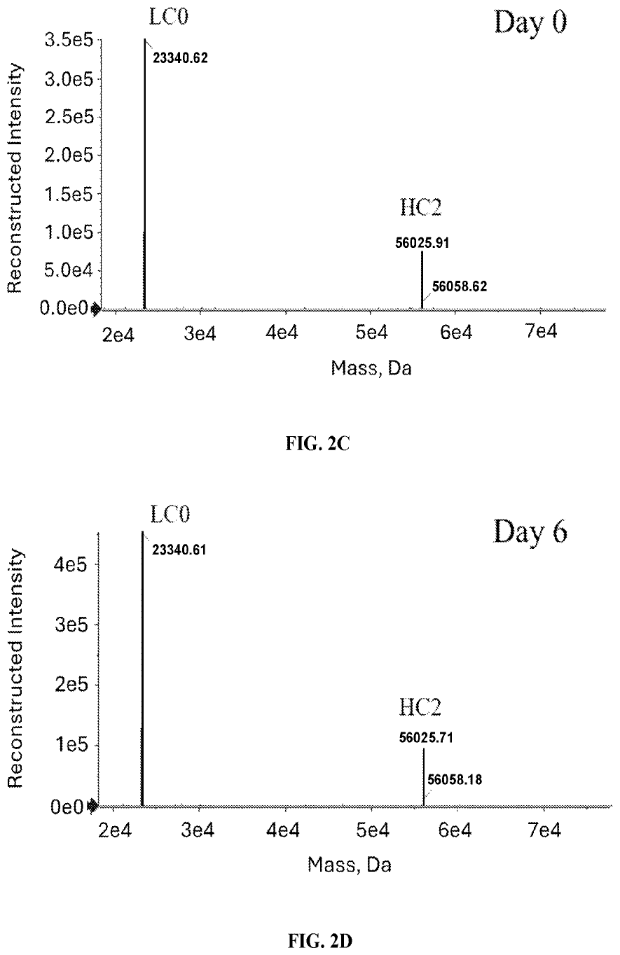

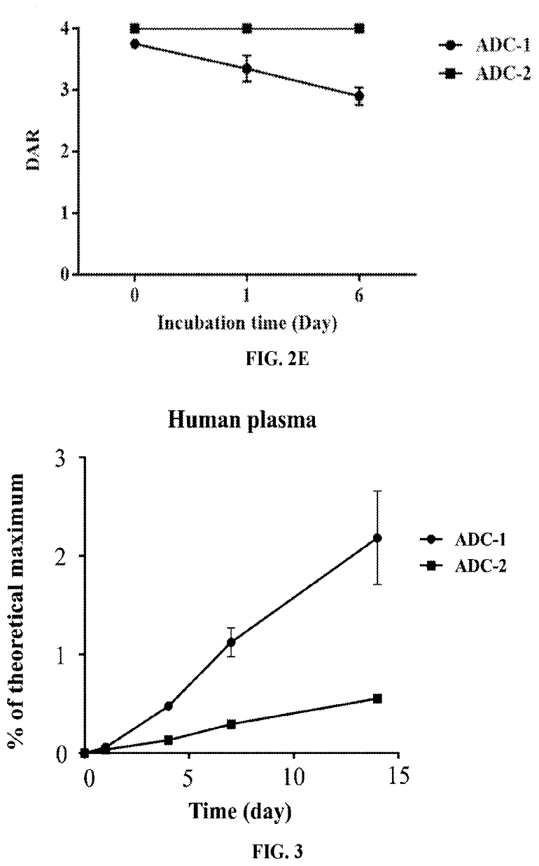

The deconvoluted mass spectrum of ADC-1 and ADC-2 incubated in HSA for 0 or 144 hours are shown in FIGS. 2A-2D. After 144 hours (six days) of incubation, the maleimide linker-payload loss was observed in ADC-1 (FIGS. 2A-2B). However, no extra peaks corresponding to HC2 degradation products were detected in ADC-2 (FIGS. 2C-2D). According to the DAR formula, a time-dependent decrease in the average DAR was observed in ADC-1 (FIG. 2E). The results indicated ADC-2 didn't undergo deconjugation via a retro-Michael reaction.

### 5-4. Measurement of In Vitro Human Plasma Stability

R4702-MCCA-PEG₂₄-ADC (ADC-1) and R4702-DBCO-PEG₂₄-ADC (ADC-2) were added to the pooled human plasma (prepared by heparin) to a final concentration of 200 μg/mL and incubated at 37° C. for 0, 24, 96, 168, or 336 hours in a shaking water bath. For determination of payload release, the incubated samples, along with internal standard (IS), were deproteinized using acetonitrile (ACN) and analyzed by LC-MS/MS. The percentage of theoretical maximum was calculated by the amount of released payload/theoretical amount of payload on DAR4 ADC×100%.

The stability of ADC-1 and ADC-2 in human plasma was monitored over 14 days at 37° C. Based on the proportion of the released payload in the theoretical amount of payload (DAR 4), the released payload percentage in ADC-1 and ADC-2 increased over time, as shown in FIG. 3. After 14 days of incubation in human plasma, the released payload percentage of ADC-1 and ADC-2 were around 2.2% and 0.6%. This result indicated that the ADC-2 released less payload than ADC-1 in human plasma.

### Example 6. In Vitro Cytotoxicity Assay of the ADCs (ADC-1 to ADC-4)

Tumor cells (2×10³ cells/well) were seeded in 96 well plates and treated with ADCs for 6 days. CellTiter-Glo® Reagent (Cat. G7572, Promega) was prepared by adding CellTiter-Glo® Buffer into lyophilized CellTiter-Glo® Substrate. Reconstituted CellTiter-Glo® Reagent was added into the culture medium with cells at 1:1 ratio after treatment for 6 days. The plate was placed on an orbital shaker for 2 minutes to induce cell lysis and then incubated at room temperature for 10 minutes before recording the luminescent signals by Luminometer. The viability of each treated sample was compared to the non-treated control. The IC₅₀ of each ADC was calculated by Prism.

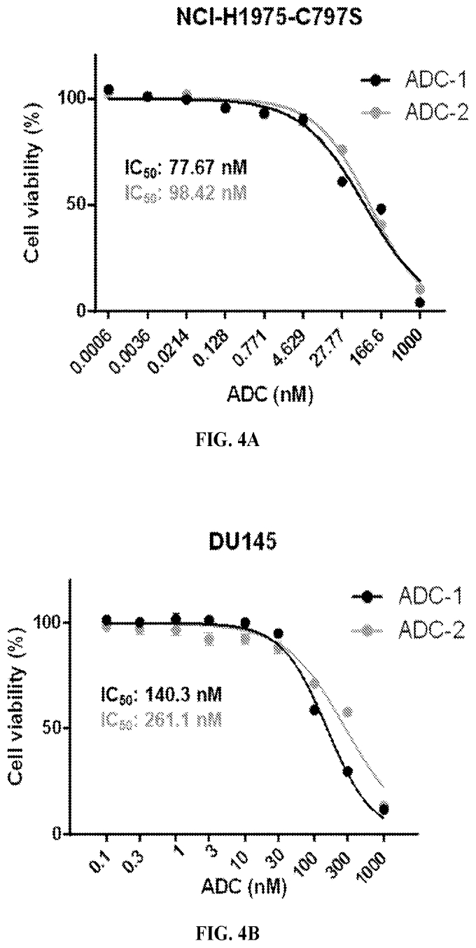

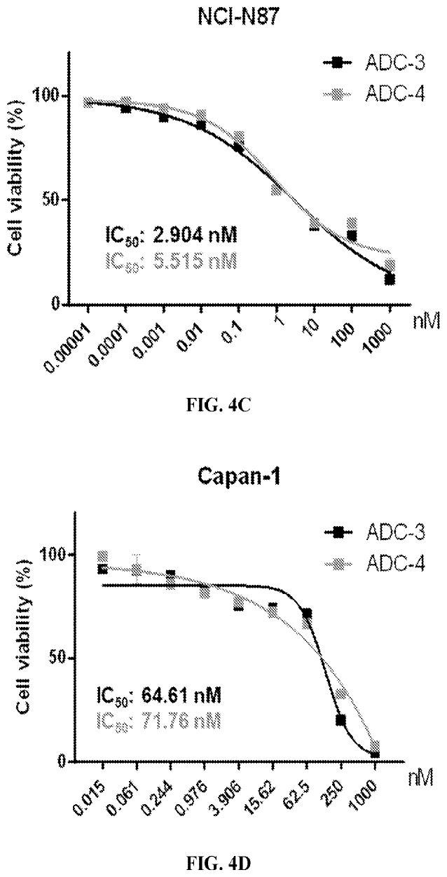

The in vitro antitumor efficacy of ADCs in several tumor cell lines were evaluated by cytotoxicity assay (FIGS. 4A-4D). The IC₅₀ of R4702-MCCA-PEG₂₄-ADC (ADC-1) and R4702-DBCO-PEG₂₄-ADC (ADC-2) in NCI-H1975-C797S lung cancer cells (ATCC, CRL-5908) were similar (77.64 and 98.42 nM). In DU145 prostate cancer cells (ATCC, HTB-81), the IC₅₀ of ADC-1 was slightly lower than ADC-2 (140.3 and 261.1 nM). Similar results were observed in TX05-MCCA-PEG₂₄-ADC (ADC-3) and TX05-DBCO-PEG₂₄-ADC (ADC-4). The IC₅₀ of ADC-3 was slightly lower than ADC-4 in NCI-N87 gastric cells (ATCC, CRL-5822) (2.904 and 5.515 nM), while ADC-3 and ADC-4 showed similar IC₅₀ in Capan-1 pancreatic cancer cells (ATCC, HTB-79) (64.61 and 71.76 nM). These data suggest that site-specific glycan-conjugated ADCs (ADC-2 and ADC-4) exhibited similar or slightly reduced cytotoxicity than cysteine conjugated ADCs (ADC-1 and ADC-3).

### Example 7. In Vivo Therapeutic Efficacy Study of the ADCs (ADC-1 to ADC-2) in NCI-H1975-C797S Lung Carcinoma Cell-Derived Xenograft

NCI-H1975-C797 human lung cancer cells (ATCC, CRL-5908) were used to evaluate the in vivo therapeutic efficacy of R4702-MCCA-PEG₂₄-ADC (ADC-1) and R4702-DBCO-PEG₂₄-ADC (ADC-2). Female BALB/c nude mice were housed in specific pathogen-free condition. Mice were acclimated for at least 3 days before the study initiation. The food (LabDiet 5010, PMI, USA) and water (sterile RO water) were provided ad libitum throughout the whole study period. All animal studies were approved by the Institutional Animal Care and Use Committee at National Laboratory Animal Center in Taiwan.

Tumor cells were washed and re-suspend in PBS. Viable cells (1×10⁷ cells/mouse) were mixed with the same volume of Matrigel (Cat. 356234, BD) and subcutaneously injected into right flank of female BALB/c nude mice (200 μL/mouse). Tumor-bearing mice were divided into different groups when the average tumor volume reached 150-200 mm³. ADCs or vehicle control were treated as a single dose thorough tail vein injection. The day of administration was denoted as Day 1. Tumor growth and mouse body weight were monitored twice weekly until Day 22. The therapeutic efficacy of ADCs was evaluated as Tumor Growth Inhibition (TGI). The TGI was calculated by the following formula: TGI (%)=[1−(Ti−T1)/(Ci−C1)]×100%. Ti and Ci indicate the mean tumor volume in the treatment groups and the vehicle group, respectively, at the end of the study (Day 22), while T1 and C1 indicate the mean tumor volumes in the treatment group and the vehicle group, respectively, at the beginning of test item administration. The experimental design, test articles, dose concentrations, dosing frequencies, route of administration and animal numbers are listed in Table 33.

TABLE 33
Dosing regimen and sampling

| | Route and | Dose of | Volume of | |
|---|---|---|---|---|
| | administration | injection | injection | Animal |
| Groups | of test item | (mg/kg) | (mL/kg) | Number |
| Vehicle | IV | — | 5 | 5 |
| ADC-1 (10 mg/kg) | IV | 10 | 5 | 5 |
| ADC-2 (10 mg/kg) | IV | 10 | 5 | 5 |
| ADC-1 (3 mg/kg) | IV | 3 | 5 | 5 |
| ADC-2 (3 mg/kg) | IV | 3 | 5 | 5 |

IV means intravenous injection.

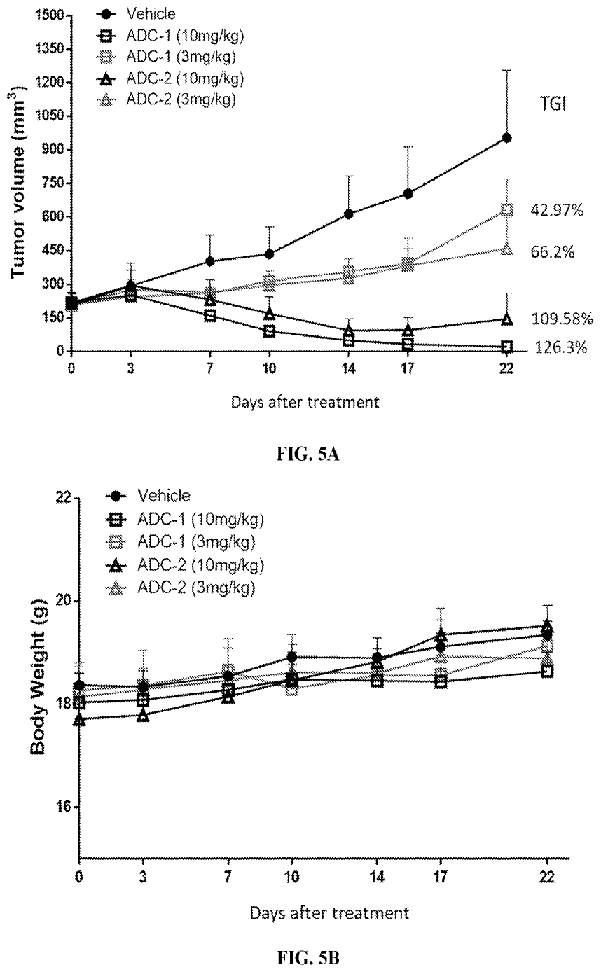

The in vivo efficacy of ADC-1 and ADC-2 were evaluated by NCI-H1975-C797S lung cancer xenograft mouse model. NCI-H1975-C797S cancer cells were implanted into BALB/c nude mice. The tumor growth (FIG. 5A) and mouse body weight (FIG. 5B) were recorded throughout the study. The tumor growth inhibition (TGI) was used to evaluate antitumor efficacy. TGI was calculated by comparing treated groups to vehicle control based on the tumor size on Day 1 and 22. Both ADC-1 and ADC-2 treated at 10 mg/kg exhibited similar excellent antitumor efficacies (TGI>100%, P=0.24). At 3 mg/kg, ADC-1 and ADC-2 showed similar partial inhibition of tumor growth (TGI: 42.7% and 66.2%, respectively, P=0.37). The results suggest that site-specific glycan-conjugated ADC-2 exhibited similar antitumor efficacy as cysteine-conjugated ADC-1.

### Example 8. Synthesis of Drug-Linker Compounds with Different PEG Lengths

### 8-1. Preparation of BCN-GGVA-Hydra-PAB-PEG₂₄-Exatecan (Also Termed DL-8), BCN-GGVA-Hydra-PAB-PEG₆-Exatecan (Also Termed DL-10), BCN-GGVA-Hydra-PAB-PEG₁₂-Exatecan (Also Termed DL-11), and BCN-GGVA-Hydra-PAB-PEG₄₈-Exatecan (Also Termed DL-12)

### Step 1:

A solution of (2R)-hydroxy (4-nitrophenyl) acetic acid (1.0 g, 5.07 mmol) in anhydrous CH₂Cl₂ (25 mL), AcCl (0.54 mL, 7.61 mmol) and pyridine (1.23 mL, 15.22 mmol) were added dropwise sequentially. The resulting mixture was stirred at room temperature for 2-3 hours. 0.1N HCl_{(aq)} (50 mL) was added, and the mixture solution was extracted with CH₂Cl₂ (50 ml×3). The combined organic layers were washed with saturated brine and then dried with MgSO₄. The organic solvent was filtered and concentrated in vacuo at 30-35° C. The crude PNB(COOH)—OAc was dried in high vacuum and then used in the next step without further purification.

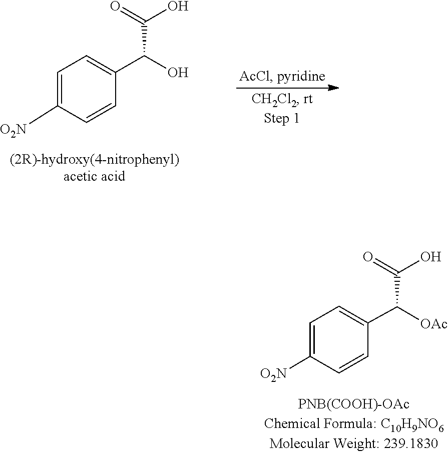 [SMILES: O=C(O)[C@H](O)c1ccc(C[N+](=O)[O-])cc1 ; CC(=O)O[C@@H](C(=O)O)c1ccc([N+](=O)[O-])cc1]

### Step 2:

Anhydrous DCM (11 mL) and anhydrous MgSO₄ (1.9 g, 15.74 mmol) were mixed in a reactor and stirred, then H₂SO₄ (0.21 mL, 3.78 mmol) was added. The reaction mixture was stirred at room temperature for 15 minutes. PNB(COOH)—OAc (753.0 mg, 3.14 mmol) and BuOH (1.49 mL, 17.741 mmol) in anhydrous CH₂Cl₂ (3 mL) was added into the previous solution at room temperature and then the mixture solution was stirred overnight. After the reaction was completed, it was quenched by adding saturated NaHCO_{3(aq)} (15 mL) slowly. The mixture solution was extracted with CH₂Cl₂ (15 mL×3) and the combined organic layers were washed with saturated brine and then dried with MgSO₄. The organic solvent was filtered and concentrated in vacuo at 30-35° C. The crude product was purified by a flash silica gel column (Hexanes/EA=5/1) to obtain 790.2 mg of PNB(COOtBu)-OAc with 85% yield. ¹H NMR (600 MHZ, CDCl₃) δ 8.23 (dd, 2H, J=6.6, 1.8 Hz), 7.65 (q, 2H, J=6.6, 1.8 Hz), 5.89 (s, 1H), 2.21 (s, 3H), 1.38 (s, 9H).

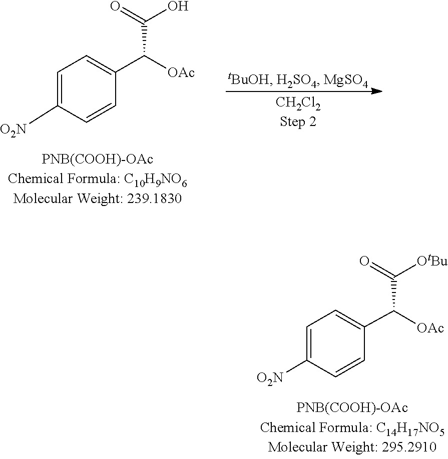 [SMILES: CC(=O)O[C@@H](C(=O)O)c1ccc(C[N+](=O)[O-])cc1 ; CC(=O)O[C@@H](C(=O)OC(C)(C)C)c1ccc([N+](=O)[O-])cc1]

### Step 3:

A solution of PNB(COOtBu)-OAc (366.6 mg, 1.24 mmol) in MeOH (24.8 mL) was cooled to 0-4° C. After 5 min, 5.4 M NaOMe in MeOH (22.9 L, 0.12 mmol) was added slowly. The reaction mixture was stirred at 0-4° C. for 30-40 minutes. After the reaction was completed, saturated NH₄Cl_{(aq)} (30 mL) was added to quench the reaction, and then the mixture solution was extracted with CH₂Cl₂ (20 ml×3). The combined organic layers were dried with MgSO₄, filtered, and then concentrated in vacuo at 30-35° C. The crude product was purified by a flash silica gel column (Hexanes/EA/CH₂Cl₂=7/1/1 to 5/1/1) to obtain 273.1 mg of PNB(COOtBu)-OH with 87% yield.

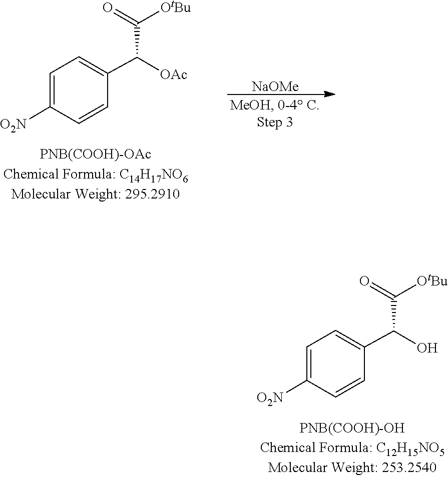 [SMILES: CC(=O)O[C@@H](C(=O)OC(C)(C)C)c1ccc([N+](=O)[O-])cc1 ; CC(C)(C)OC(=O)[C@H](O)c1ccc([N+](=O)[O-])cc1 ; "NaOMe"]

### Step 4:

To a solution of PNB(COOtBu)-OH (273.1 mg, 1.08 mmol) in MeOH (10.8 mL), 10% Pd/C (41.0 mg, 15 wt %) was added. The reaction was stirred at room temperature with H₂ balloon overnight. After the reaction was completed, the reaction mixture was passed through celite (3.0 g) and the celite was washed with MeOH (10 mL×3). The desired fraction was concentrated in vacuo at 30-35° C. The crude PAB (COOtBu)-OH was dried in high vacuum and then used in the next step without further purification.

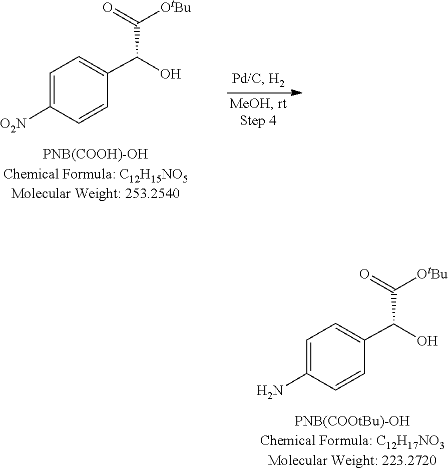 [SMILES: CC(C)(C)OC(=O)[C@H](O)c1ccc([N+](=O)[O-])cc1 ; CC(C)(C)OC(=O)[C@H](O)c1ccc(N)cc1]

### Step 5:

PAB (COOtBu)-OH (99.9 mg, 0.45 mmol) and Boc-GGVA-OH (216.2 mg, 0.54 mmol) were dissolved in anhydrous CH₂Cl₂ (2.0 ml) and IPA (2.0 mL). EEDQ (166.1 mg, 0.67 mmol) was added. The resulting mixture was stirred at room temperature overnight. After the reaction was completed, the reaction mixture was concentrated in vacuo at 30-35° C. The crude product was purified by a flash silica gel column (EA/CH₂Cl₂=99/1 to 97/3, then CH₂Cl₂/MeOH=9/1) to obtain 198.5 mg of N-DT-0025 with 73% yield.

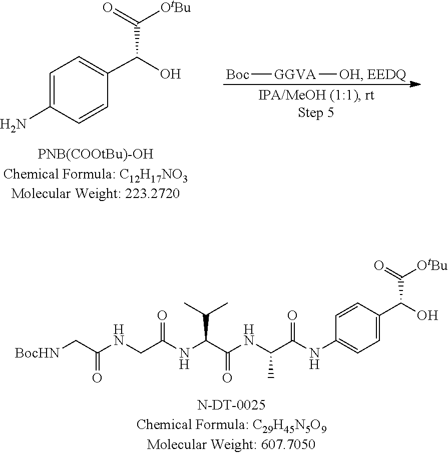

### Step 6:

N-DT-0025 (316.9 mg, 0.52 mmol) and Bis(4-nitrophenyl) carbonate (396.6 mg, 1.30 mmol) were dissolved in anhydrous DMF/CH₂Cl₂ (6.3 mL, v/v=1/9). DIPEA (0.36 mL, 2.09 mmol) was added into the reaction solution. The reaction was stirred at room temperature overnight. After the reaction was completed, the reaction mixture was concentrated in vacuo at 30-35° C. The crude product was purified by a flash silica gel column (EA/CHCl₃=99/1, then CH₂Cl₂/MeOH=15/1) to obtain 275.3 mg of N-DT-0024 with 68.3% yield. ¹H NMR (600 MHZ, CDCl₃) δ 8.34 (d, 2H, J=9.0 Hz), 7.71 (d, 2H, J=8.5 Hz), 7.51 (d, 2H, J=9.0 Hz), 7.45 (d, 2H, J=8.5 Hz), 5.83 (s, 1H), 4.47 (q, 1H, J=7.1 Hz), 4.19 (d, 1H, J=6.6 Hz), 3.97 (d, 1H, J=16.5 Hz), 3.89 (d, 1H, J=16.5 Hz), 3.72 (d, 2H, J=5.3 Hz), 2.19-2.13 (m, 1H), 1.47 (d, 3H, J=7.2 Hz), 1.44 (s, 9H), 1.42 (s, 9H), 1.00 (dd, 6H, J=14.3, 6.8 Hz).

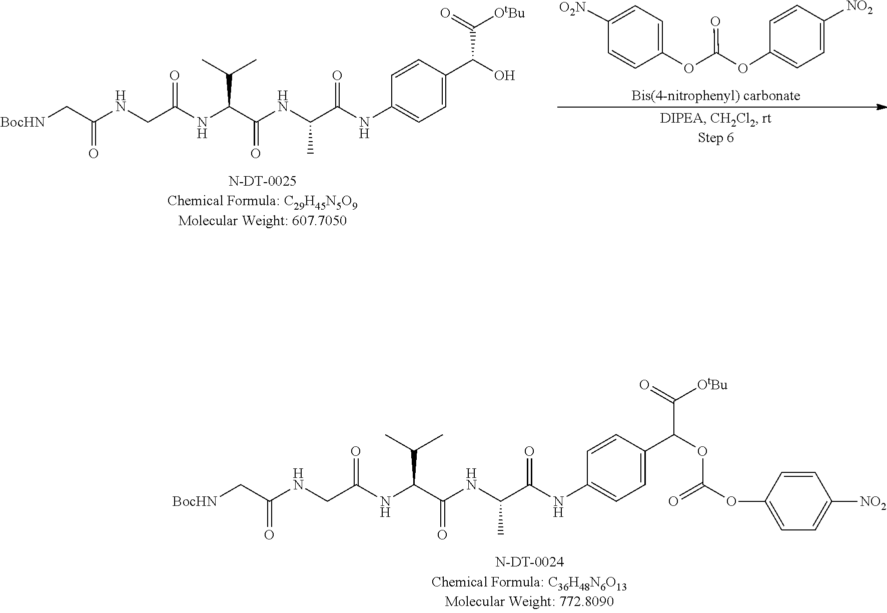 [SMILES: CC(C)[C@H](NC(=O)CNC(=O)CNC(=O)OC(C)(C)C)C(=O)N[C@@H](C)C(=O)Nc1ccc([C@@H](O)C(=O)OC(C)(C)C)cc1 ; O=C(Oc1ccc([N+](=O)[O-])cc1)Oc1ccc([N+](=O)[O-])cc1 ; CC(C)[C@H](NC(=O)CNC(=O)CNC(=O)OC(C)(C)C)C(=O)N[C@@H](C)C(=O)Nc1ccc(C(OC(=O)Oc2ccc([N+](=O)[O-])cc2)C(=O)OC(C)(C)C)cc1]

### Step 7:

To a suspension of exatecan mesylate (34.0 mg, 0.06 mmol) and N-DT-0024 (49.4 mg, 0.06 mmol) in anhydrous DMF (0.64 mL), DIPEA (33.4 μL, 0.19 mmol) was added at room temperature. The suspension became a clear brown solution within 5 minutes. This mixture was stirred at room temperature overnight. After the reaction was completed, the reaction mixture was added dropwise into a stirring TBME (6.4 mL) to get precipitate. The solids were collected by filtration and followed by high-vacuum drying to obtain crude N-PM-0023 (65.4 mg, 95.7% yield). This crude product was used in the next step without further purification.

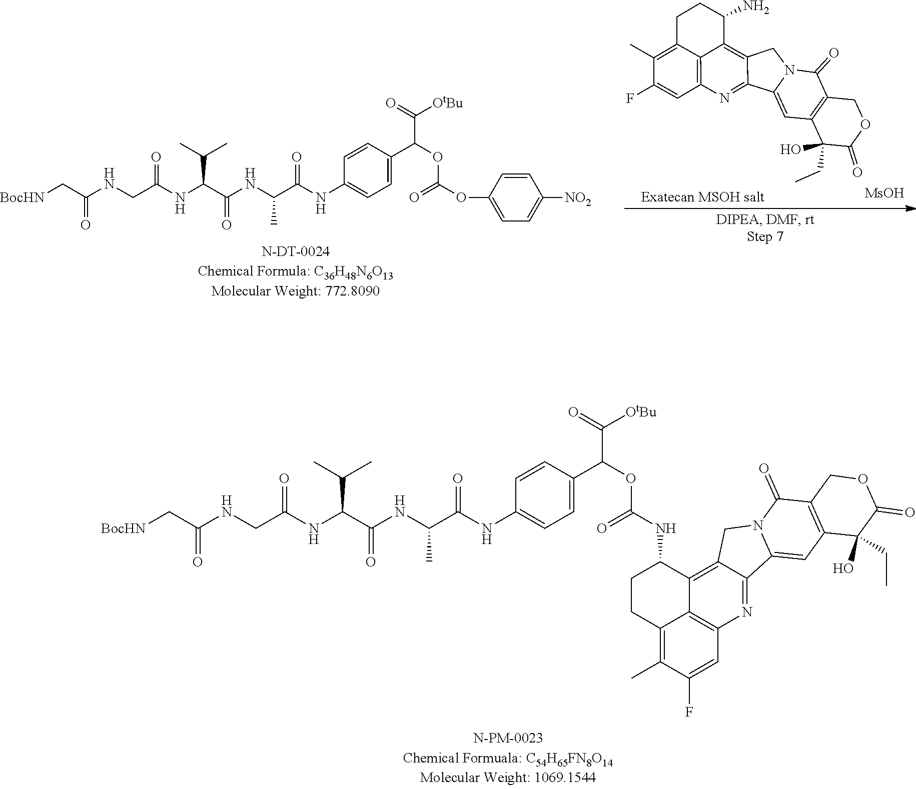 [SMILES: "MsOH" ; CC(C)[C@H](NC(=O)CNC(=O)CNC(=O)OC(C)(C)C)C(=O)N[C@@H](C)C(=O)Nc1ccc(C(OC(=O)Oc2ccc([N+](=O)[O-])cc2)C(=O)OC(C)(C)C)cc1 ; CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)OC(C(=O)OC(C)(C)C)c1ccc(NC(=O)[C@H](C)NC(=O)[C@@H](NC(=O)CNC(=O)CNC(=O)OC(C)(C)C)C(C)C)cc1)CC3]

### Step 8:

A stirring suspension of N-PM-0023 (83.4 mg, 0.08 mmol) in DCM (1.40 mL) was cooled to 0° C. TFA (6.88 mL) was added at 0° C. slowly. The reaction mixture was stirred at 0-4° C. for 20-24 hours. After the reaction was completed, the reaction mixture was added dropwise into a stirring TBME (20.9 mL) to get precipitate. The solids were collected by filtration and followed with high vacuum drying to obtain crude N-PM-0024 (73.4 mg, 91.7% yield). This crude product was used in the next step without further purification.

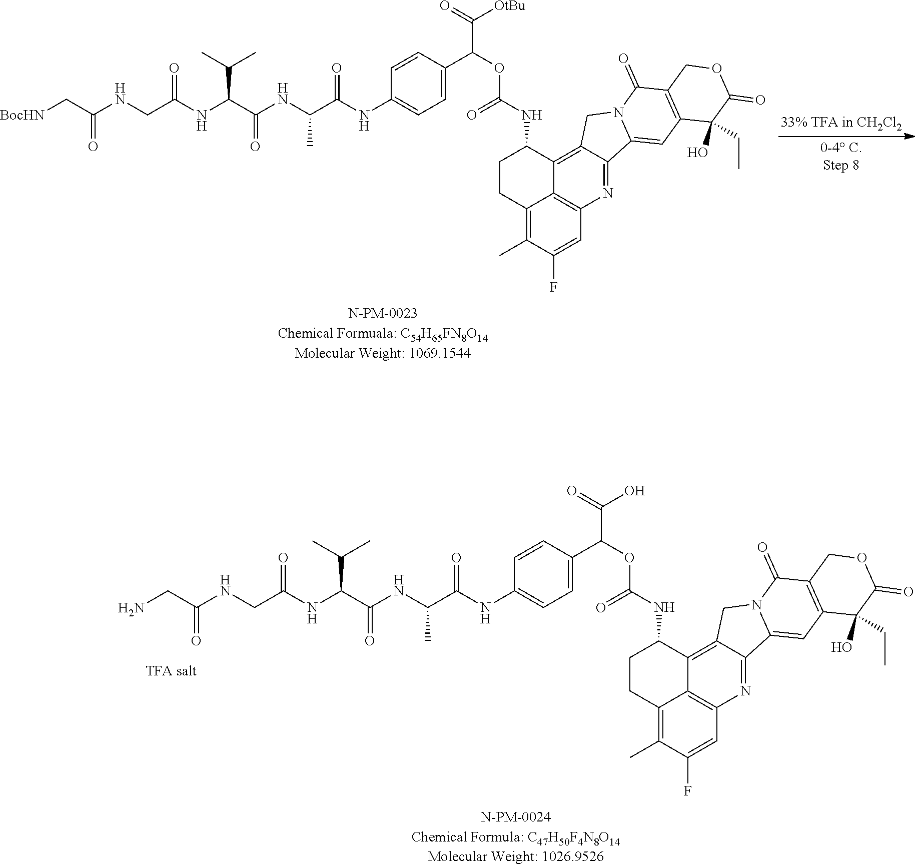 [SMILES: CC[C@@]1(O)C(=O)OCc2c1cc1n(c2=O)Cc2c-1nc1cc(F)c(C)c3c1c2[C@@H](NC(=O)OC(C(=O)OC(C)(C)C)c1ccc(NC(=O)[C@H](C)NC(=O)[C@@H](NC(=O)CNC(=O)CNC(=O)OC(C)(C)C)C(C)C)cc1)CC3 ; CC[C@@]1(O)C(=O)OCc2c1cc1n(c2=O)Cc2c-1nc1cc(F)c(C)c3c1c2[C@@H](NC(=O)OC(C(=O)O)c1ccc(NC(=O)[C@H](C)NC(=O)[C@@H](NC(=O)CNC(=O)CN)C(C)C)cc1)CC3]

### Step 9:

N-PM-0024 (56.9 mg, 0.05 mmol) and BCN—OSu (16.2 mg, 0.05 mmol) were dissolved in anhydrous DMF (1.1 mL). Et₃N (23.2 μL, 0.16 mmol) was added into the reaction solution at room temperature. The reaction mixture was stirred at room temperature overnight. After the reaction was completed, the reaction mixture was added dropwise into a stirring TBME (11 mL) to get precipitate. The solids were collected by filtration and followed by high-vacuum drying to obtain crude N-PM-0025 (60.2 mg, 99.7% yield). This crude product was used in the next step without further purification.

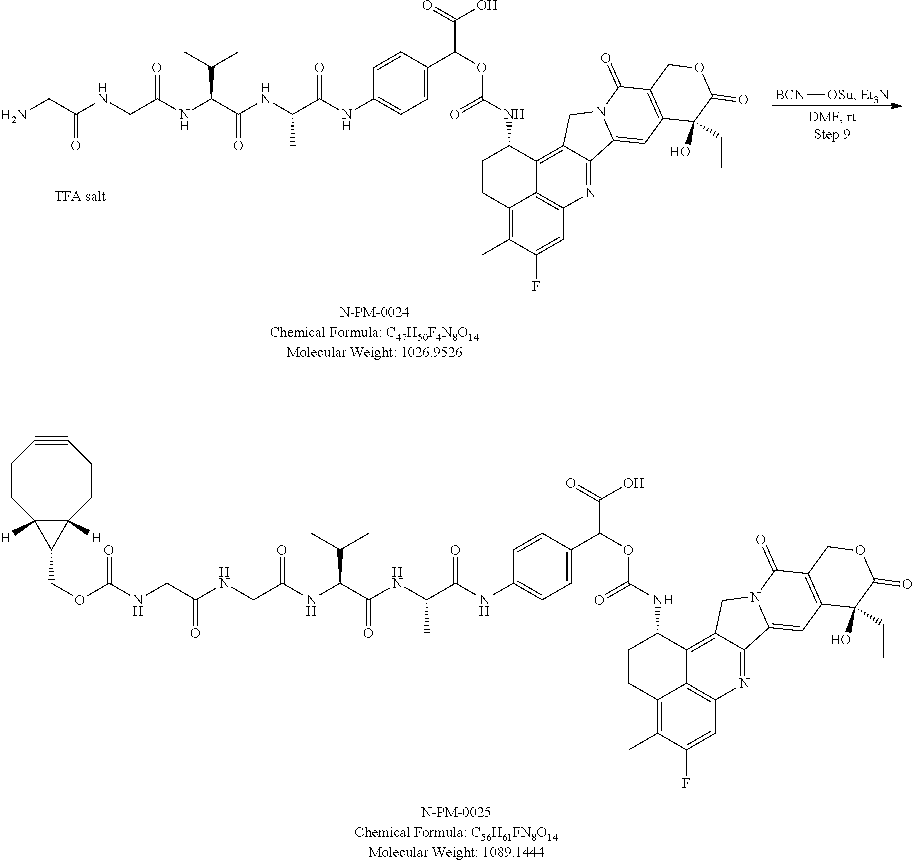

### Step 10:

N-PM-0025 (1 eq) and m-PEG-amine with various PEG length (2 eq) and HATU (1.2 eq) were dissolved in anhydrous DMF (0.05 M). NMM (5 eq) was added. The reaction mixture was stirred at room temperature overnight. After the reaction was completed, the resulting mixture was purified with preparative HPLC to obtain DL-8 (with PEG₂₄), DL-10 (with PEG₆), DL-11 (with PEG₁₂), and DL-12 (with PEG₄₈). DL-8 (BCN-GGVA-Hydra-PAB-PEG₂₄-Exatecan) was obtained with 31.5% yield. HRMS (ESI) m/z found [(M+2H)/2]⁺, 1080.0534 C₁₀₅H₁₆₁FN₉O₃₇²⁺, required 1079.5488. DL-10 (BCN-GGVA-Hydra-PAB-PEG₆-Exatecan) was obtained with 35.2% yield. HRMS (ESI) m/z found [(M+2Na)/2]⁺, 705.8000 C₆₉H₈₈FN₉O₁₉Na₂²⁺, required 705.7988. DL-11 (BCN-GGVA-Hydra-PAB-PEG₁₂-Exatecan) was obtained with 42.7% yield. HRMS (ESI) m/z found [(M+2Na)/2]⁺, 837.8782 C₈₁H₁₁₂FN₉O₂₅Na₂²⁺, required 837.8774. DL-12 (BCN-GGVA-Hydra-PAB-PEG₄₈-Exatecan) was obtained with 47.1% yield. HRMS (ESI) m/z found [(M+4H)/4]⁺, 804.6855 C₁₅₃H₂₅₇FN₉O₆₁⁴⁺, required 804.6870.

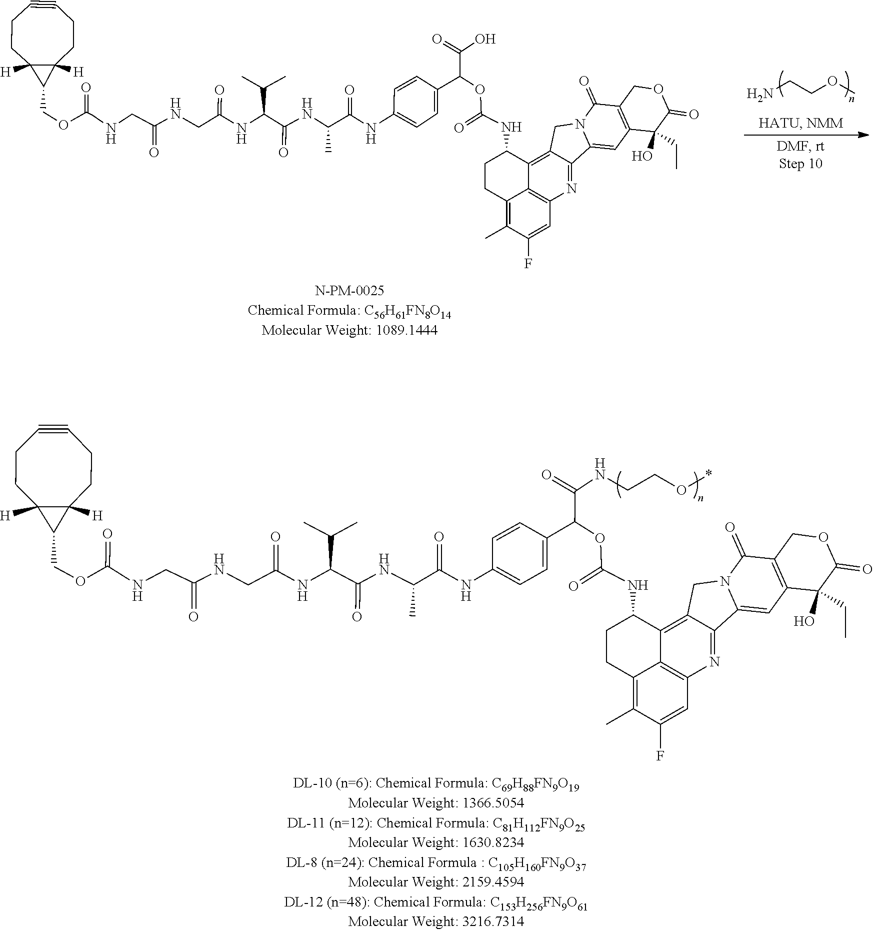

TABLE 34
HPLC condition
(Column: YMC-Actus Trait C18 250 × 20 mm, 5 μL, 12 nm)

| Gradient: | | | |
|---|---|---|---|
| Time | Flow (ml/min) | H₂O | ACN |
| 0 min | 10 | 90% | 10% |
| 1 min | 20 | 70% | 30% |
| 3 min | 20 | 55% | 45% |
| 4 min | 20 | 54% | 46% |
| 6 min | 20 | 53% | 47% |
| 8 min | 20 | 52% | 48% |
| 10 min | 20 | 52% | 48% |
| 14 min | 20 | 50% | 50% |
| 15 min | 20 | 46% | 54% |
| 16 min | 20 | 42% | 58% |
| 17 min | 20 | 38% | 62% |
| 17.1 min | 20 | 10% | 90% |
| 22 min | 20 | 10% | 90% |

### 8-2. Preparation of BCN-GGVA-Hydra-PAB-Exatecan (Also Termed DL-13)

### Steps 1 and 2:

N-PM-0018 (41.2 mg, 0.047 mmol), Fmoc-GG-OH (16.8 mg, 0.047 mmol) and HATU (21.6 mg, 0.057 mmol) were dissolved in anhydrous DMF (0.95 mL). NMM (15.7 μL, 0.142 mmol) was added. The reaction was stirred at room temperature for 4-5 hours. After the reaction was completed, CH₂Cl₂/MeOH (1.56 mL, v/v=1/1) was added. Et₂NH (155.1 μL) was added at room temperature. Then the reaction mixture was stirred at room temperature overnight. After the reaction was completed, the resulting mixture was purified with preparative HPLC to obtain 21.7 mg of GGVA_PAR_Exa with 52.6% yield

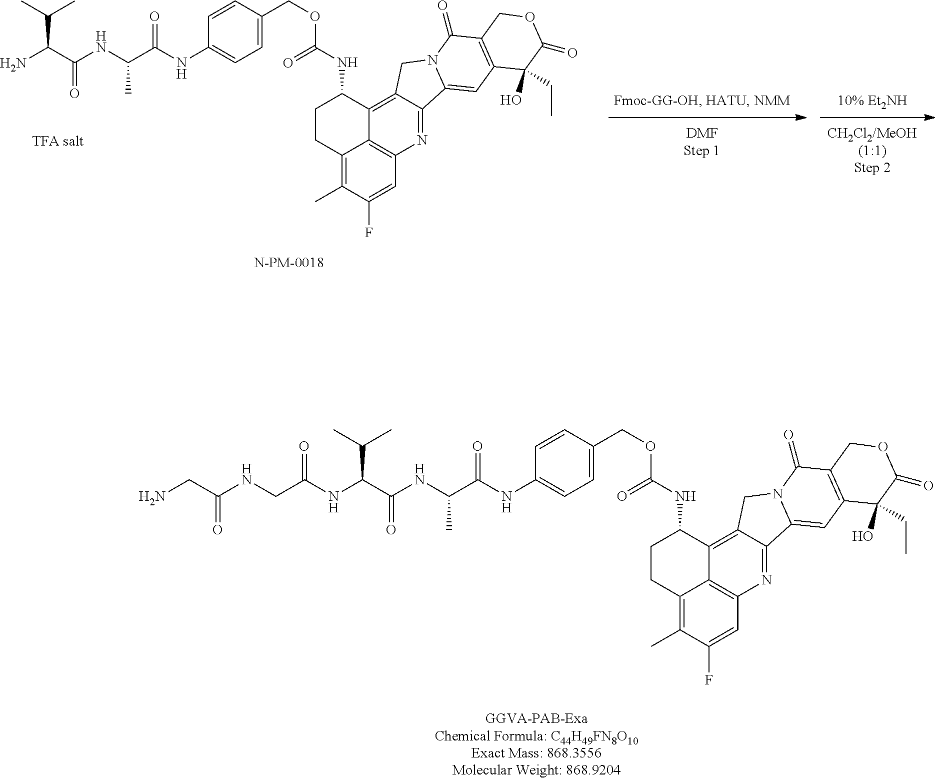 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](N)C(C)C)cc1)CC3 ; CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)CNC(=O)CN)C(C)C)cc1)CC3]

TABLE 35
HPLC condition
(Column: YMC-Actus Trait C18 250 × 20 mm, 5 μL, 12 nm)

| Gradient: | | | |
|---|---|---|---|
| Time | Flow (ml/min) | H₂O | ACN |
| 0 min | 10 | 90% | 10% |
| 1 min | 20 | 80% | 20% |
| 5 min | 20 | 70% | 30% |
| 20 min | 20 | 50% | 50% |
| 25 min | 20 | 10% | 90% |
| 30 min | 20 | 10% | 90% |

### Step 3:

GGVA-PAB-Exa (21.6 mg, 0.025 mmol) and BCN—Osu (7.3 mg, 0.025 mmol) were dissolved in anhydrous DMF (0.5 mL). Et₃N (10.4 μL, 0.075 mmol) was added. The reaction mixture was stirred at room temperature overnight. After the reaction was completed, the resulting mixture was purified with preparative HPLC to obtain 11.7 mg of DL-13 (BCN-GGVA-Hydra-PAB-Exatecan) with 45.0% yield.

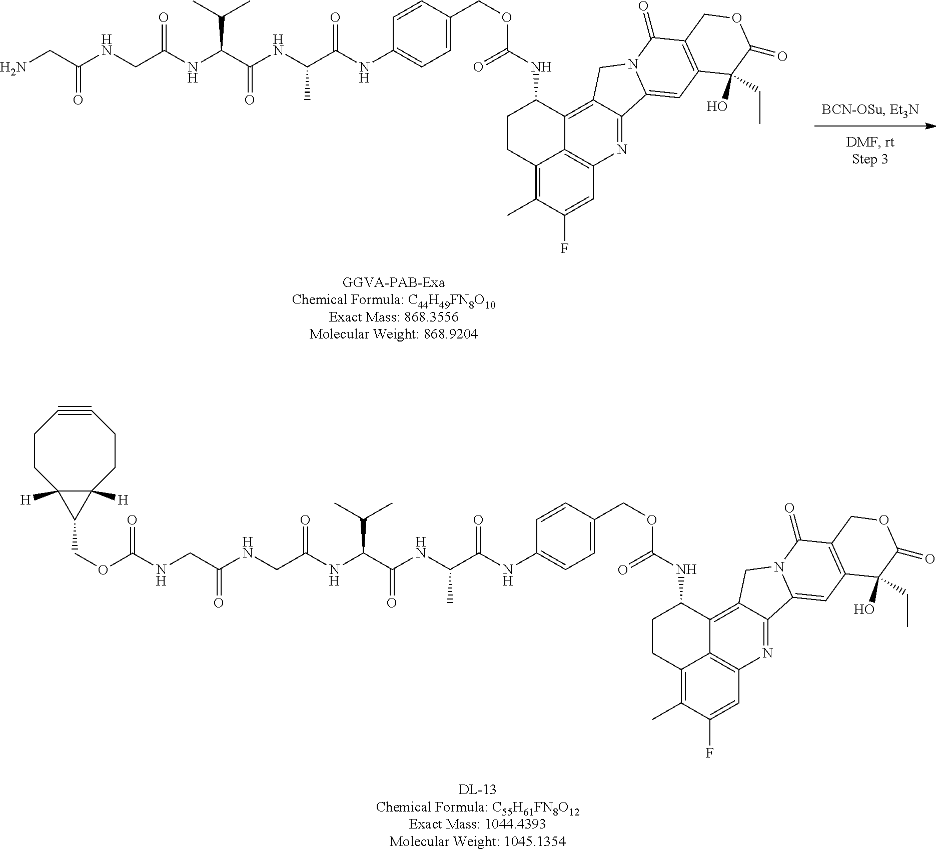 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)CNC(=O)CN)C(C)C)cc1)CC3 ; CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)CNC(=O)CNC(=O)OC[C@@H]2[C@@H]4CCC#CCC[C@@H]42)C(C)C)cc1)CC3]

TABLE 36
HPLC condition
(Column: YMC-Actus Trait C18 250 × 20 mm, 5 μL, 12 nm)

| Gradient: | | | |
|---|---|---|---|
| Time | Flow (ml/min) | H₂O | ACN |
| 0 min | 10 | 90% | 10% |
| 1 min | 20 | 80% | 20% |
| 20 min | 20 | 50% | 50% |
| 25 min | 20 | 10% | 90% |
| 35 min | 20 | 10% | 90% |

### Example 9. ADC Preparation and In Vitro 3D Cytotoxicity Assay of ADCs with Different PEG Lengths (ADC-5 to ADC-9)

### 9-1. Preparation of ADCs with Different PEG Lengths (ADC-5 to ADC-9)

BCN linker-payload (8-16 eq) was dissolved in propylene glycol/DMSO (1 mg linker-payload/50 μL propylene glycol/DMSO; 0-50% (v/v) propylene glycol in DMSO) and then added slowly to R4702-(NSCT-di-N3)₂ solution (antibody concentration 5 mg/mL in 20 mM NaOAc, pH 5.0), and the mixture was shaken at 25° C. for 6-24 hours. After the conjugation was completed, the crude ADC was further purified by using Spectrum® Hollow Fiber Filter Modules (buffer: 20 mM NaOAc, pH 5.0) to afford R4702-BCN-ADC. ADC was adjusted to around 10 mg/mL and sterilized by passing through ProMax™ Syringe Filter (PVDF, 0.22 μm). The DAR value of the ADC was determined by hydrophilic interaction chromatography (HIC).

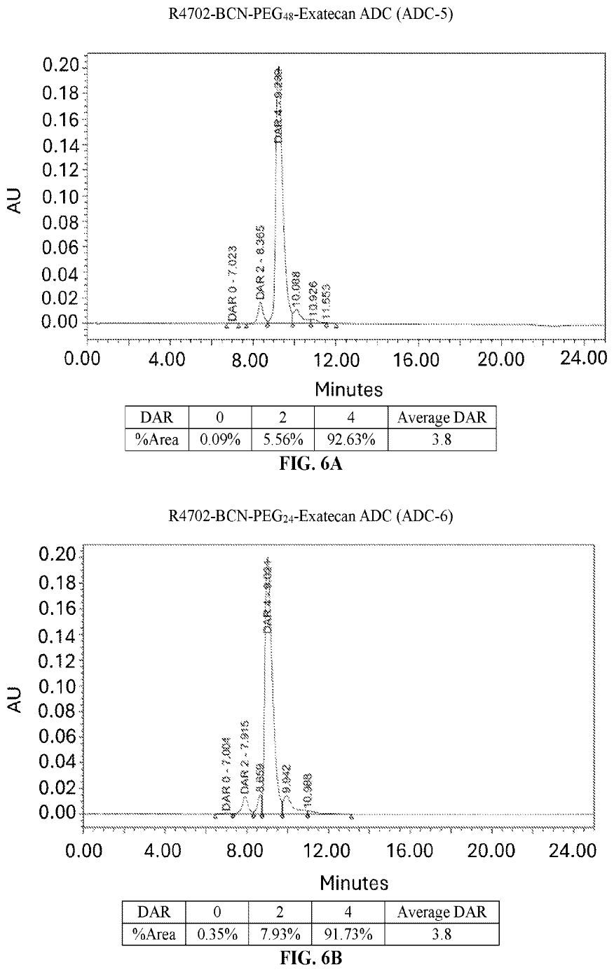

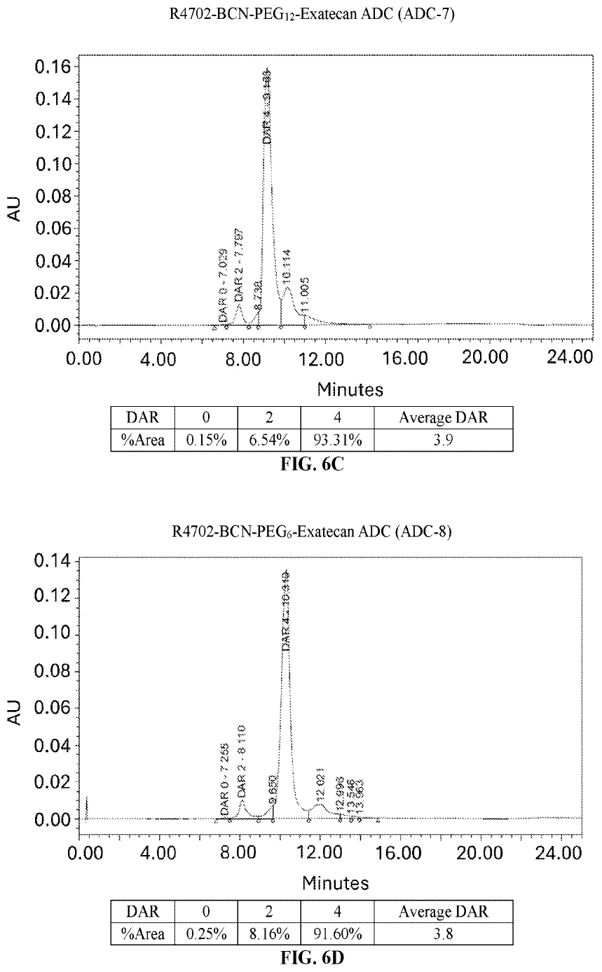

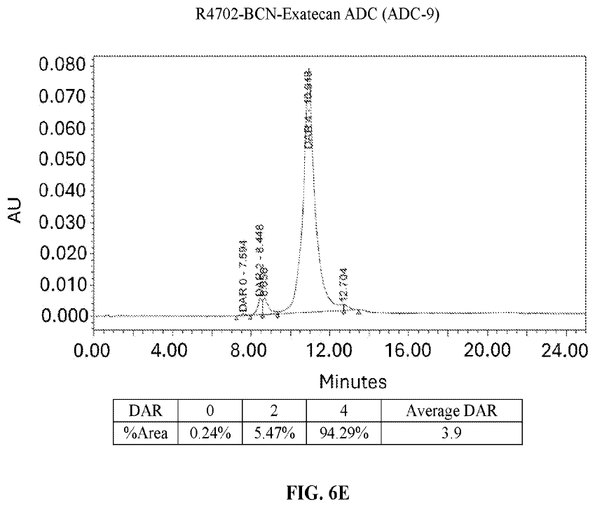

FIGS. 6A-6E indicated the HIC results of five R4702 (anti-TROP2) ADCs with different PEG lengths. FIG. 6A indicated the DAR distribution of R4702-BCN-PEG₄₈-Exatecan ADC (ADC-5) was 0.09% for DAR0, 5.56% for DAR2, and 92.63% for DAR4. The average DAR was 3.8. FIG. 6B indicated the DAR distribution of R4702-BCN-PEG₂₄-Exatecan ADC (ADC-6) was 0.35% for DAR0, 7.93% for DAR2, and 91.73% for DAR4. The average DAR was 3.8. FIG. 6C indicated the DAR distribution of R4702-BCN-PEG₁₂-Exatecan ADC (ADC-7) was 0.15% for DAR0, 6.54% for DAR2, and 93.31% for DAR4. The average DAR was 3.9. FIG. 6D indicated the DAR distribution of R4702-BCN-PEG₆-Exatecan ADC (ADC-8) was 0.25% for DAR0, 8.16% for DAR2, and 91.60% for DAR4. The average DAR was 3.8. FIG. 6E indicated the DAR distribution of R4702-BCN-Exatecan ADC (ADC-9) was 0.24% for DAR0, 5.47% for DAR2, and 94.29% for DAR4. The average DAR was 3.9.

### 9-2. In Vitro 3D Cytotoxicity Assay of ADCs with Different PEG Lengths (ADC-5 to ADC-9)

Tumor cells (NCI-N87 shVOID: high TROP2 expressing gastric carcinoma; NCI-N87 shTROP2: low TROP2 expressing gastric carcinoma; NCI-H1975: medium TROP2 expressing non-small cell lung carcinoma, NSCLC) were purchased from ATCC and further infected with adenovirus. The cells were seeded at a density of 1-2×10² cells per well in 50 μL of complete medium supplemented with 1% Matrigel (Corning, New York, USA) in ultralow attachment 96-well plates (Thermo Scientific, Waltham, Massachusetts, USA). The cells were allowed to form compact 3D aggregates. After six days, the spheroid size was assessed by measuring the perimeter using ImageJ software (NIH, Bethesda, Maryland, USA), with sizes ranging between 200-300 μm².

Spheroids were then treated with various ADCs at the indicated concentrations. Following an additional six days of incubation, cell viability was measured using the CellTiter-Glo® 3D cell viability assay (Promega Corporation, Madison, Wisconsin, USA), and the luminescent signal was recorded according to the manufacturer's instructions.

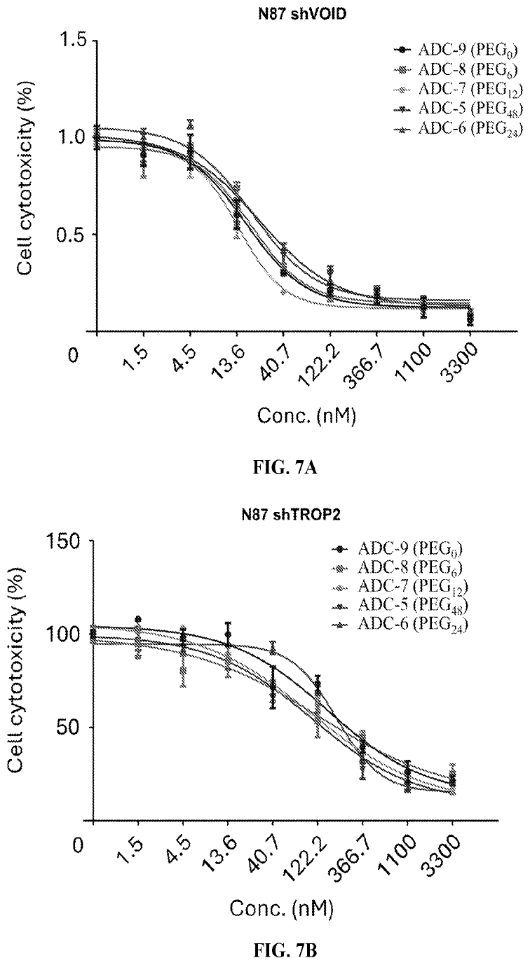

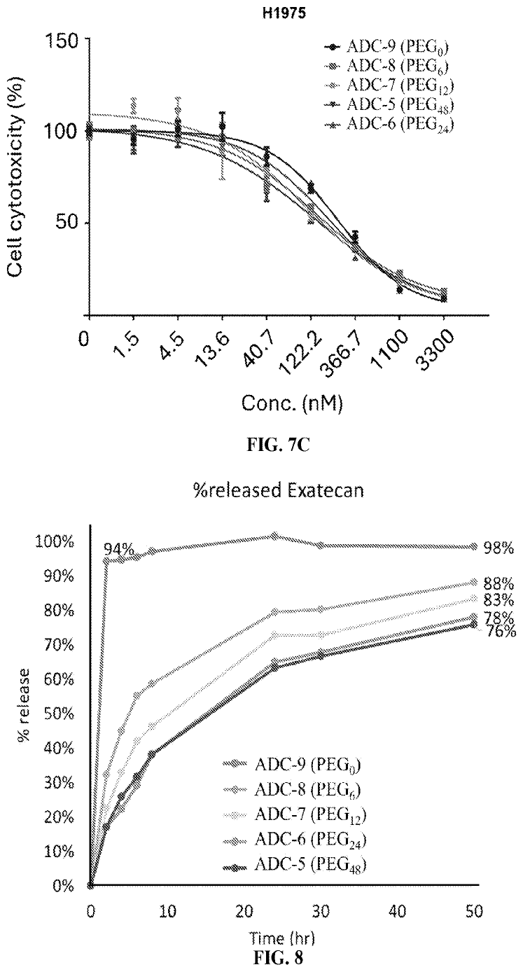

The in vitro therapeutic efficacy of R4702 (anti-TROP2) ADCs were evaluated by 3D cytotoxicity assay (FIGS. 7A-7C). The EC₅₀ values observed in NCI-N87 sh VOID (high TROP2) were ranging from 14.11 to 23.16 nM (FIG. 7A). The EC₅₀ values in NCI-N87 shTROP2 (low TROP2) were ranging from 104.7 to 193.7 nM (FIG. 7B). The EC₅₀ values in NCI-H1975 (medium TROP2) were ranging from 119.4 to 237.6 nM (FIG. 7C). These data indicated that the EC₅₀ values for the ADCs with different PEG lengths were similar (Table 37).

TABLE 37
The EC50 values for the ADCs with different PEG lengths

| | NCI-N87 sh VOID | NCI-N87 shTROP2 | NCI-N87 sh VOID |
|---|---|---|---|
| | EC50 nM | EC50 nM | EC50 nM |
| ADC | (high TROP2) | (low TROP2) | (medium TROP2) |
| ADC-5 | 23.16 | 107.7 | 142.4 |
| (PEG₄₈) | | | |
| ADC-6 | 19.54 | 193.7 | 180.1 |
| (PEG₂₄) | | | |
| ADC-7 | 14.11 | 104.7 | 119.4 |
| (PEG₁₂) | | | |
| ADC-8 | 18.8 | 125.8 | 145.1 |
| (PEG₆) | | | |
| ADC-9 | 16.8 | 157.2 | 237.6 |
| (PEG₀) | | | |

### 9-3. The Payload Release Efficiency of ADCs with Different PEG Lengths

This study was conducted to evaluate how the length of PEG in the linker-payload would affect drug release from ADC. The study was evaluated by digesting ADCs with different PEG lengths (PEG₀ to PEG₄₈) using cathepsin B and qualifying the released exatecan (as an example of drug unit) by LC-UV. 25 μg of ADCs were treated with 10 μg/mL of activated cathepsin B in digestion buffer for exatecan cleavage at 37° C. The reaction mixture was sampled at 2, 4, 6, 8 24, 30 and 50 hours after adding the activated cathepsin B into the ADCs. A C₁₈ column (Symmetry 5 μm, 2.1×150 mm Waters) was applied for this analysis. Samples were eluted with ACN/water gradient at a flow rate of 0.5 mL/minute, and the chromatogram was monitored at 263 nm.

FIG. 8 indicated the results of exatecan release efficiency of ADCs with different PEG lengths after cathepsin B cleavage. The % released exatecan was derived by the ratio of the released exatecan to theoretical exatecan on DAR4 ADC. As shown in the figure, ADC-9 (no PEG) was completely digested by cathepsin B and released most of exatecan after two hours of digestion (98%). Other ADCs had not reached the highest exatecan release until fifty hours of digestion. The exatecan release efficiency of ADCs, from the highest to the lowest, was 88% for ADC-8 (PEG₆), 83% for ADC-7 (PEG₁₂), 78% for ADC-6 (PEG₂₄), and 76% for ADC-5 (PEG₄₈). ADCs with PEG₂₄ or PEG₄₈ linker-payloads had similar payload release efficiency. The results demonstrated that the length of PEG may affect its ability to protect linker-payloads from destruction. Since cathepsin B cleavage might mimic the in-vivo circumstance, the results suggested that ADCs with longer PEG moieties would enhance stability of ADCs in blood circulation of a subject.

### Example 10. In-Vivo Efficacy Comparison of ADCs with Different PEG Lengths (ADC-5 to ADC-9)

### 10.1 Test Substances and Dosing Patterns

(a) ADC-9: PEG₀ (9.8 mg/mL)
(b) ADC-8: PEG₆ (8.33 mg/mL)
(c) ADC-7: PEG₁₂ (10.0 mg/mL)
(d) ADC-6: PEG₂₄; (also termed OBI-902) (9.9 mg/mL)
(e) ADC-5: PEG₄₈ (9.9 mg/mL)

TABLE 38
Study Design and sampling

| | Route and | Dose of | Volume of | |
|---|---|---|---|---|
| | administration | injection | injection | Animal |
| Groups | of test item | (mg/kg) | (mL/kg) | number |
| G1: Vehicle | IV | — | 5 | 6 |
| G2: ADC-9 (PEG₀) | IV | 3 | 5 | 6 |
| G3: ADC-8 (PEG₆) | IV | 3 | 5 | 6 |
| G4: ADC-7 (PEG₁₂) | IV | 3 | 5 | 6 |
| G5: ADC-6 (PEG₂₄) | IV | 3 | 5 | 6 |
| G6: ADC-5 (PEG₄₈) | IV | 3 | 5 | 6 |

Intravenous injection (IV): Volume of injection was 5 mL/kg

### 10.2 Cell Line: NCI-H1975 Cells (TROP2-Expressing Human Non-Small Cell Lung Cancer Cells) (ATCC, CRL-5908)

### 10.3 Animal

(a) Species: Mus musculus
(b) Strain: CAnN·Cg-Foxn1^{nu}/CrlBltw (BALB/c nude)
(c) Source: BioLasco Taiwan
(d) Sex: Female
(e) Age at the initiation of the study: 7 weeks
(f) Body weight range at the start of the study: 15-25 g
(g) Animal grouping: The mice were divided into six groups and each group contained five mice. A total of thirty-six mice were involved in the study.

### 10.4 Equipment and Materials

(a) Biosafety cabinet (NUAIRE/NU-620-400)
(b) Electronic balance (CROMTECH/YP30002)
(c) Isolated positive/negative pressure validated cage housing system (TECNIPLAST/Blue Line)
(d) Vernier (METROLOGY/EC-9001V)
(e) Matrigel (BD/Cat. No.: 356234)

### 10.5 Methods

### (a) Establishment of a Xenograft Mouse Model

Subcutaneous inoculation of tumor cells: 5×10⁶ NCI-H1975 cells were mixed with the equal volume of matrigel (volume ratio 1:1) (Corning, 354248, Lot No.: 0261002). Subcutaneous injection volume was 100 L/mouse.

### (b) Route and Administration of Test Articles:

The first dosing day was denoted as Day 1 when average tumor volume reaches 150-200 mm³. All test articles (test item 1 to 5) or reference items (sodium citrate solution) were intravenously administered to the mice on Day 1. The injection was performed using insulin syringe with the dosage of 3 mg/kg, and the injection volume was 5 mL/kg.

### (c) Body Weight Measurement

Measurement started from the next day of tumor inoculation. Animal body weight was measured and recorded three times per week.

### (d) Tumor Growth Inhibition Rate Calculation

Tumor volumes were used to calculate tumor growth inhibition (TGI) rates according to the following formula: TGI (%)=[1−(Ti−T1)/(Ci−C1)]×100%, where Ti and Ci indicate the mean tumor volume in the treatment groups and vehicle group, respectively, at the end of the study (Day 23), whereas T1 and C1 indicate the mean tumor volumes in the treatment group and vehicle group, respectively, at the beginning of test item administration (Day 1).

### (e) Statistical Analysis

Results were presented as mean and standard error of the mean (mean±SEM). Comparisons of all data collected for each treatment group with vehicle group was calculated using Student's t-test. p<0.05 was considered significance.

### 10.6 Results

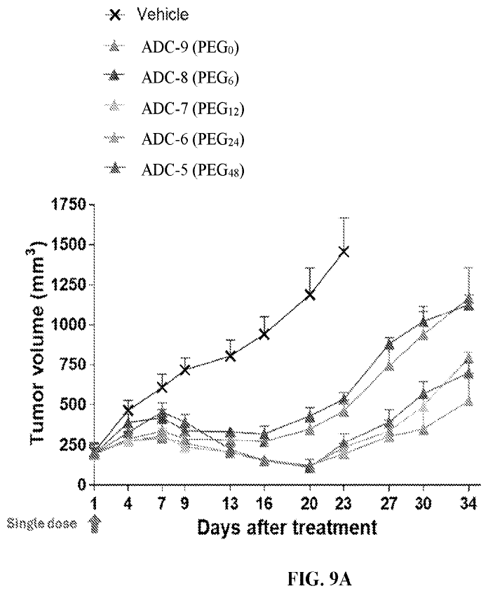

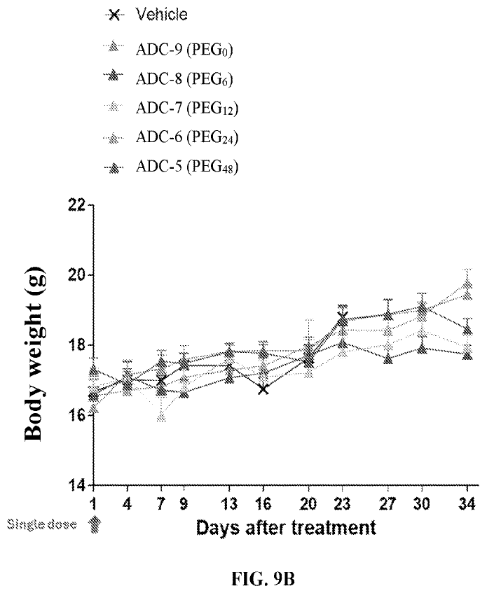

FIGS. 9A-9B indicated the tumor volume of the vehicle group was 1457±210.36 mm³ on Day 23. Until Day 34, the tumor volume of the ADC-9 (PEG₀) group was 1167.48±188.75 mm³. The tumor volume of the ADC-8 (PEG₆) group was 1121.61±61.41 mm³. The tumor volume of the ADC-7 (PEG₁₂) group was 794.48±35.46 mm³. The tumor volume of the ADC-6 (PEG₂₄) group was 543.43±173.22 mm³. The tumor volume of the ADC-5 (PEG₄₈) group was 698.70±69.64 mm³. The results demonstrated that PEG could enhance tumor suppression efficacy of ADCs (FIG. 9A). Furthermore, the maximum tumor inhibition efficacy was observed in the ADC with PEG₂₄. Besides, there was no significant difference observed in the mean body weight among groups G1 to G6 during the study period (FIG. 9B).

### Example 11. The Digestion Efficiency, Payload Release Efficiency and In-Vitro Cytotoxicity Assay Of Different Linker-Payloads [DBCO-VA-Hydra-PAB-PEG₂₄-Exatecan (DL-5), BCN-VA-Hydra-PAB-PEG₂₄-Exatecan (DL-6), DBCO-GGVA-Hydra-PAB-PEG₂₄-Exatecan (DL-7), BCN-GGVA-Hydra-PAB-PEG₂₄-Exatecan (DL-8)]

This study was conducted to evaluate how the DCBO and BCN in the linker-payloads would affect drug release. The study also included the linker-payloads with the two-amino acid spacer “GG” in the digestion comparison. The study was evaluated by digesting the linker-payloads using cathepsin B and quantifying the released exatecan by LC-UV. To perform exatecan release test by cathepsin B cleavage, 5 μg of the linker-payloads were treated with 10 μg/mL of activated cathepsin B in digestion buffer for payload cleavage at 37° C. The reaction mixture was sampled every two hours for 24 hours, and the amounts of released exatecan and residual linker-payload were analyzed by LC-UV. A C₁₈ column (Symmetry 5 μm, 2.1×150 mm Waters) was applied for this analysis. Samples were eluted with ACN/water gradient at a flow rate of 0.5 mL/min, and the chromatogram was monitored at 263 nm.

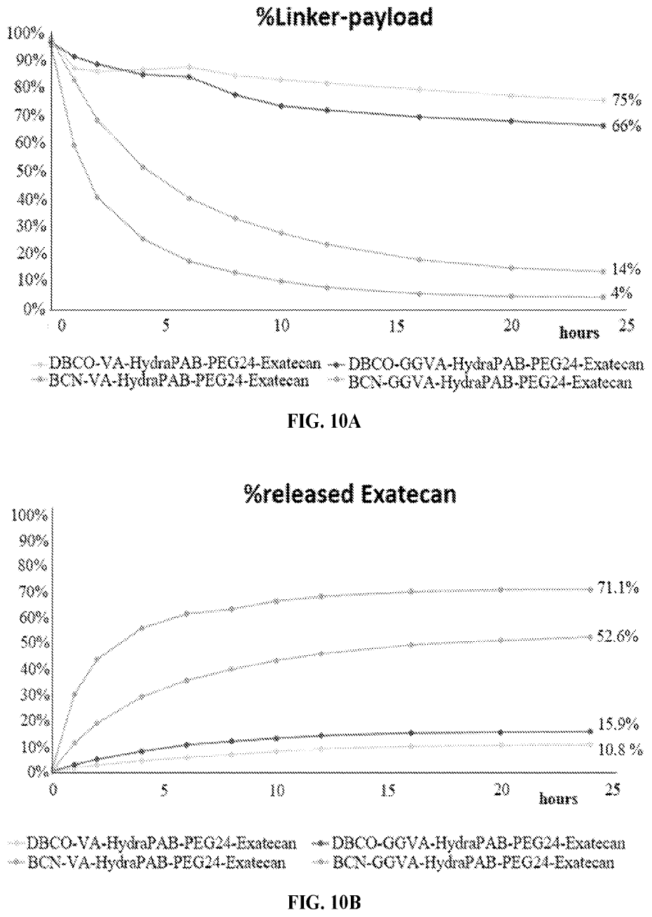

FIG. 10A indicated the digestion efficiency of different linker-payloads. The calculated percentage of linker-payloads existed (% linker-payload) after cathepsin B cleavage reaction were 75% for DBCO-VA-Hydra-PAB-PEG₂₄-Exatecan (DL-5), 66% for DBCO-GGVA-Hydra-PAB-PEG₂₄-Exatecan (DL-7), 14% for BCN-VA-Hydra-PAB-PEG₂₄-Exatecan (DL-6), and 4% for BCN-GGVA-Hydra-PAB-PEG₂₄-Exatecan (DL-8). The results demonstrated that the linker payload with BCN had better digestion efficiency. Furthermore, FIG. 10B indicated the exatecan release efficiency of different linker-payloads. The uppermost amount of released exatecan (71.1%) was observed in BCN-VA-Hydra-PAB-PEG₂₄-Exatecan (DL-6), followed by 52.6% for BCN-GGVA-Hydra-PAB-PEG₂₄-Exatecan (DL-8), 15.9% for DBCO-GGVA-Hydra-PAB-PEG₂₄-Exatecan (DL-7), and 10.8% for DBCO-GGVA-Hydra-PAB-PEG₂₄-Exatecan (DL-5). Both results demonstrated that linker-payloads with the BCN group exhibited better payload release efficiency compared to those with the DBCO group.

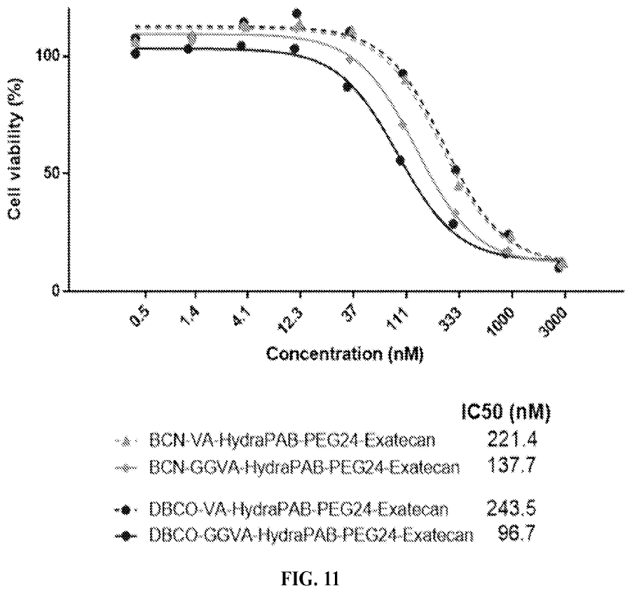

Cytotoxicity was determined using CellTiter-Glo™ luminescent assay reagent (Promega). Human SKBR-3 breast cancer cells (ATCC, HTB-30) were used in this study. The cells were plated in 96-well plates with 8E+03 cells per well and incubated at 37° C. overnight. Different linker payloads (DL-5 to DL-8) were prepared from 3000 to 0.5 nM with three-fold serial dilution in cell culture medium (McCoy's 5A medium contain 10% FBS) and then added to the plates. After incubated at 37° C. for six days, the CellTiter-Glo luciferase assay reagent was added to each well. The plate was placed on an orbital shaker for two minutes to induce cell lysis and then incubated at room temperature for ten minutes before recording the luminescent signals by Luminometer. Luminescence was determined using a microplate luminometer SpectraMax L (Molecular Devices, Sunnyvale, CA). The percentage of the cytotoxicity was calculated by dividing the non-treated cell luminescence minus experimental cell luminescence by the non-treated cell luminescence and multiplying by 100. IC₅₀ was determined by plotting x (concentration in nM)-y (drug cytotoxicity in %) and fitting the data in a 4PL nonlinear regression model by PRISM 6 Software. FIG. 11 indicated the IC₅₀ of DL-5 (DBCO-VA-Hydra-PAB-PEG₂₄-Exatecan) was 243.5 nM, the IC₅₀ of DL-6 (BCN-VA-Hydra-PAB-PEG₂₄-Exatecan) was 221.4 nM, the IC₅₀ of DL-8 (BCN-GGVA-Hydra-PAB-PEG₂₄-Exatecan) was 137.7 nM, and the IC₅₀ of DL-7 (DBCO-GGVA-Hydra-PAB-PEG₂₄-Exatecan) was 96.7 nM. The IC₅₀ data for the different linker payloads were similar.

### Example 12. The Payload Release Efficiency and In-Vitro Cytotoxicity Assay of ADCs with Different Bioorthogonal Groups [R4702-BCN-GGVA-Hydra-PAB-PEG₂₄-Exatecan (ADC-10), R4702-DBCO-GGVA-Hydra-PAB-PEG₂₄-Exatecan (ADC-11)]

This study was to evaluate the effect of different bioorthogonal groups (BCN or DBCO groups) of ADCs on payload release. The study was conducted by digesting R4702-BCN-GGVA-Hydra-PAB-PEG₂₄-Exatecan (also termed ADC-10) or R4702-DBCO-GGVA-Hydra-PAB-PEG₂₄-Exatecan (also termed ADC-11) using cathepsin B and monitoring the released exatecan (an example of drug unit) by LC-UV. 25 μg of ADCs were treated with 10 μg/mL of activated cathepsin B in digestion buffer for exatecan cleavage at 37° C. The reaction mixture was sampled at 2, 4, 6, 8, 18, 24, 48 and 57 hours after adding the activated cathepsin B into ADCs. A Cortecs C₁₈ column (2.7 μm, 3.0×150 mm) was used for this analysis. Samples were eluted with ACN/water gradient at a flow rate of 0.3 mL/min, and the chromatogram was monitored at 263 nm.

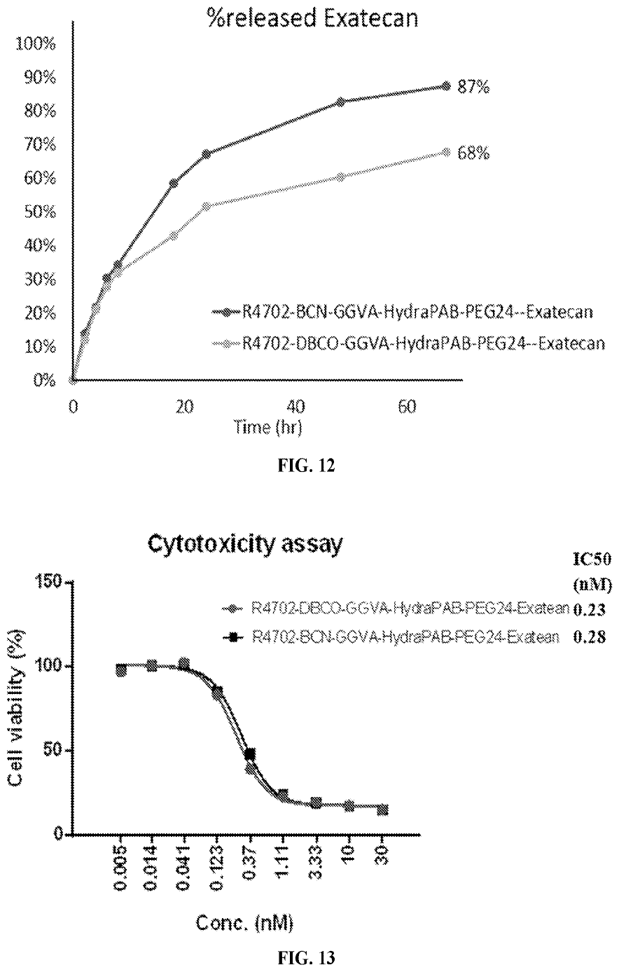

FIG. 12 indicated the results of exatecan release efficiency of ADCs after cathepsin B cleavage. The % released exatecan was derived by the ratio of the released exatecan to theoretical exatecan on ADCs (ADC-10:87% and ADC-11:68%). The results demonstrated the bioorthogonal group of ADCs would affect the payload digestion efficiency and releasing.

Cytotoxicity was determined using CellTiter-Glo™ Luminescent assay reagent (Promega). Human BxPC-3 pancreatic adenocarcinoma cancer cells (ATCC, CRL-1687) were used in this study. The cells were plated in 96-well plates with 8E+03 cells per well and incubated at 37° C. overnight. Different bioorthogonal groups of ADCs (ADC-10 and ADC-11) were prepared from 30 to 0.005 nM with three-fold serial dilution in cell culture medium (McCoy's 5A medium contain 10% FBS) and then added to the plates. After incubated at 37° C. for six days, the CellTiter-Glo luciferase assay reagent was added to each well. The plate was placed on an orbital shaker for two minutes to induce cell lysis and then incubated at room temperature for ten minutes before recording the luminescent signals by Luminometer. Luminescence was determined using a microplate luminometer SpectraMax L (Molecular Devices, Sunnyvale, CA). The percentage of the cytotoxicity was calculated by dividing the non-treated cell luminescence minus experimental cell luminescence by the non-treated cell luminescence and multiplying by 100. IC₅₀ was determined by plotting x (concentration in nM)-y (drug cytotoxicity in %) and fitting the data in a 4PL nonlinear regression model by PRISM 6 Software. FIG. 13 indicated the IC₅₀ of ADC-10 (R4702-DBCO-GGVA-Hydra-PAB-PEG₂₄-Exatecan) was 0.23 nM, and the IC₅₀ of ADC-11 (R4702-BCN-GGVA-Hydra-PAB-PEG₂₄-Exatecan) was 0.28 nM. The IC₅₀ data for the ADCs with different bioorthogonal groups were similar.

Unless defined otherwise, all technical and scientific terms and any acronyms used herein have the same meanings as commonly understood by one of ordinary skill in the art in the field of this invention. Although any compositions, methods, kits, and means for communicating information similar or equivalent to those described herein can be used to practice this invention, the preferred compositions, methods, kits, and means for communicating information are described herein.

All references cited herein are incorporated herein by reference to the full extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

1. A linker-payload of Formula (III): 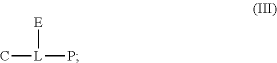
wherein:
E is a hydrophilic moiety comprising polyethylene glycol (PEG), polysarcosine (pSar), poly lactic-co-glycolic acid (PLGA), poly(glycerols) (PGs), poly(oxazolines) (POX), poly(hydroxypropyl methacrylate) (PHPMA), poly(2-hydroxyethyl methacrylate) (PHEMA), poly(N-(2-hydroxypropyl) methacrylamide) (HPMA), poly(vinylpyrrolidone) (PVP), poly(N,N-dimethyl acrylamide) (PDMA), poly(N-acryloylmorpholine) (PACM), saccharides, or any combination thereof;
C is a bioorthogonal group for conjugation to an antibody or an antigen-binding fragment thereof;
L is a linker unit comprising a protease-cleavable peptide moiety, a glycosidase-cleavable sugar moiety, a pH sensitive moiety, or a hydrolysable moiety; and
P is a payload selected from a drug unit or a probe unit.

2. The linker-payload according to claim 1, having a structure of Formula (X): 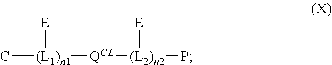
wherein:
L₁ and L₂ are the same or different connector units, wherein the connector unit comprises an amino acid or a functional group defined as 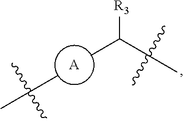
wherein A is an aromatic group;
R₃ is linked to E and is selected from —C(O)—, —C(O)O—, —C(O)NH—, alkyl-O—, alkyl-NH—, alkyl-C(O)—, alkyl-C(O)—O—, alkyl-C(O)—NH—, alkyl-NH—C(O)—, alkyl-SO₂—, alkyl-S—, alkyl-O—P(O)—O₂—, alkyl-O—C(O)—NH—, or triazole;
n1 and n2 are independently 0, 1 or 2, but n1 and n2 are not 0 at the same time; and
Q^{CL} is a cleavable unit, wherein the cleavable unit comprises the protease-cleavable peptide moiety, the glycosidase-cleavable sugar moiety, the pH sensitive moiety, or the hydrolysable moiety.

3. The linker-payload according to claim 1, wherein C is selected from a dibenzocyclooctyne (DBCO) group, a bicyclononyne (BCN) group, a alkyne group, a maleimide group, a α,β-unsaturated carbonyl group, a sulfonyl pyrimidine group, a 4-dibenzocyclooctynol (DIBO) group, a aza-dibenzocyclooctynes (DIBAC) group, a tetrazine group, a tetrazole group, a norbornene group, a cyclooctyne group, a methylcyclopropene group, an aminooxy group, a hydrazine group, an isocyanide group, an isocyanopropanoate group, a phosphine-containing thioester group, a phosphine phenolic ester group, or an alpha-halo carbonyl group.

4. The linker-payload according to claim 1, wherein P is selected from a chemotherapeutic agent, a toxin, a cytokine, a growth inhibitory agent, a protein degrader, a peptide, a radionuclide, a hormone, an anti-viral agent, an anti-bacterial agent, an immunoregulatory agent, a fluorophore, a dye, or a contrast agent.

5. The linker-payload according to claim 4, wherein the toxin is selected from pyrrolobenzodiazepine compounds or derivatives thereof, auristatin compounds or derivatives thereof, maytansinoid compounds or derivatives thereof, duocarmycin or derivatives thereof, nicotinamide phosphoribosyltransferase (NAMPT) inhibitors or derivatives thereof, tubulysin compounds or derivatives thereof, enediyne compounds or derivatives thereof, anthracycline compounds or derivatives thereof, pyrrole-based kinesin spindle protein (KSP) inhibitors or derivatives thereof, cryptophycin compounds or derivatives thereof, drug efflux pump inhibitors or derivatives thereof, sandramycin or derivatives thereof, amanitin compounds or derivatives thereof, or camptothecin compounds or derivatives thereof.

6. The linker-payload according to claim 1, wherein the structure of Formula (III) is further represented by having a structure of Formula (IV): 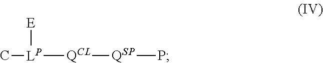
wherein:
Q^{SP} is a spacer unit comprising an aromatic group or amino methylene;
Q^{CL} is a cleavable unit, wherein the cleavable unit comprises the protease-cleavable peptide moiety, the glycosidase-cleavable sugar moiety, the pH sensitive moiety, or the hydrolysable moiety;
L^{P} is a connector unit comprising one or more amino acids.

7. The linker-payload according to claim 6, wherein E has the formula of: 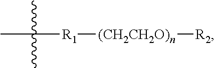
wherein the wavy line indicates the site of covalent attachment to L^{P};
R₁ is —C(O)—, —O—, —S—, —NH—, —C(O)O—, alkyl-C(O)—NH—, alkyl-NH—C(O)—, alkyl-CO₂—, alkyl-S—, or 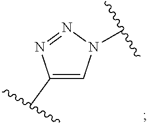 [SMILES: *c1cn(*)nn1]
R₂ is H, SO₃H, PO₃H₂, a sugar derivative, C₁-C₁₀ (hetero)alkyl group, C₃-C₁₀ (hetero)cycloalkyl group, C₂-C₁₀ alkyl-NH₂, C₁-C₁₀ alkyl-COOH, C₂-C₁₀ alkyl-NH(C₁-C₃ alkyl), C₂-C₁₀ alkyl-N(C₁-C₃ alkyl)₂, or sarcosines; and
n is an integer ranging from 2 to 72.

8. The linker-payload according to claim 6, having a structure of the following formula: 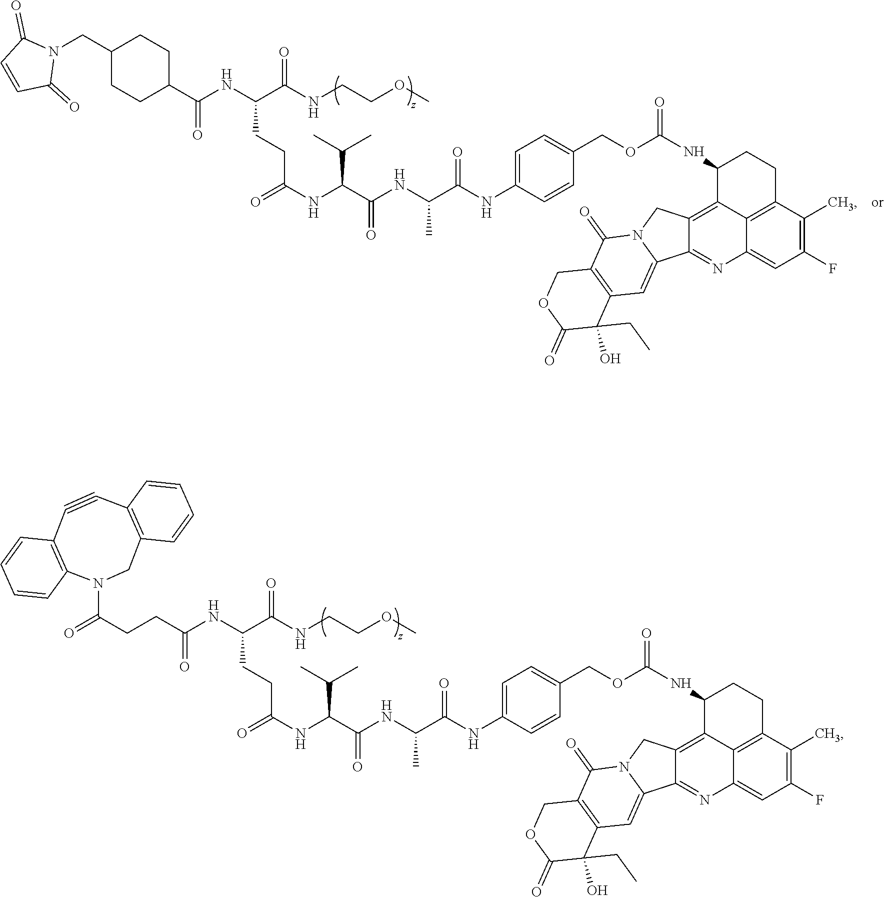
wherein z is an integer ranging from 6 to 48.

9. (canceled)

10. The linker-payload according to claim 1, wherein the structure of Formula (III) is further represented by a structure of Formula (V): 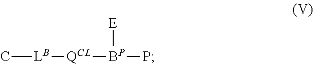
wherein:
B^{P} is a branch unit comprising a functional group defined as 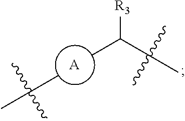
wherein A is an aromatic group;
R₃ is linked to E and is selected from —C(O)—, —C(O)O—, —C(O)NH—, alkyl-O—, alkyl-NH—, alkyl-C(O)—, alkyl-C(O)—O—, alkyl-C(O)—NH—, alkyl-NH—C(O)—, alkyl-SO₂—, alkyl-S—, alkyl-O—P(O)—O₂—, alkyl-O—C(O)—NH—, or triazole;
Q^{CL} is a cleavable unit, wherein the cleavable unit comprises the protease-cleavable peptide moiety, the glycosidase-cleavable sugar moiety, the pH sensitive moiety, or the hydrolysable moiety;
L^{B} is a bridge unit.

11. The linker-payload according to claim 10, wherein E has the formula of: 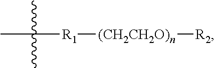
wherein the wavy line indicates the site of covalent attachment to B^{P},
R₁ is —C(O)—, —O—, —S—, —NH—, —C(O)O—, alkyl-C(O)—NH—, alkyl-NH—C(O)—, alkyl-CO₂—, alkyl-S—, or 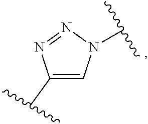 [SMILES: *c1cn(*)nn1]
R₂ is H, SO₃H, PO₃H₂, a sugar derivative, C₁-C₁₀ (hetero)alkyl group, C₃-C₁₀ (hetero)cycloalkyl group, C₂-C₁₀ alkyl-NH₂, C₁-C₁₀ alkyl-COOH, C₂-C₁₀ alkyl-NH(C₁-C₃ alkyl), C₂-C₁₀ alkyl-N(C₁-C₃ alkyl)₂, or sarcosines; and
n is an integer ranging from 2 to 72.

12. The linker-payload according to claim 10, wherein the structure of Formula (V) is further represented by a structure of the following formula: 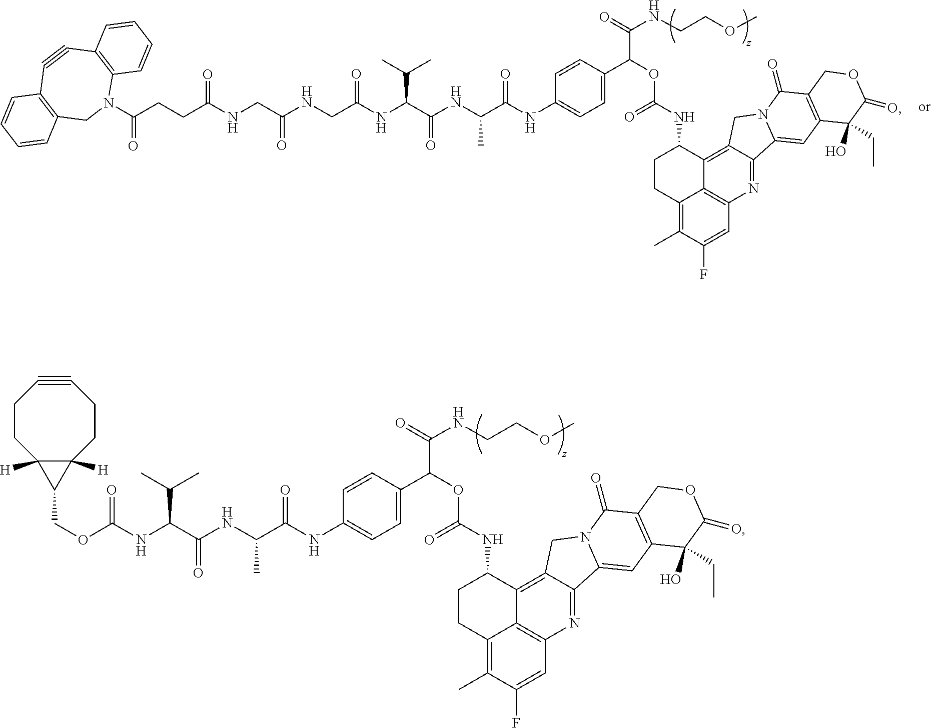
wherein z is an integer ranging from 6 to 48.

13. (canceled)

14. (canceled)

15. (canceled)

16. The linker-payload according to claim 10, wherein the structure of Formula (V) is further represented by a structure of any one of the following formulas:

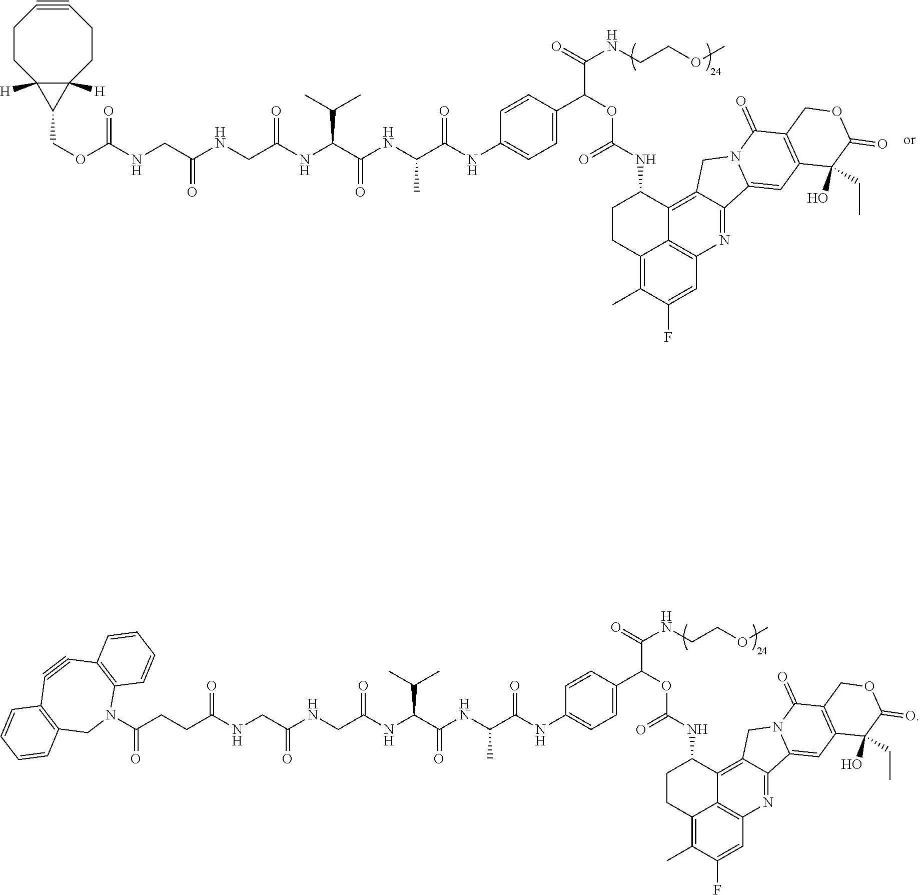

17. (canceled)

18. (canceled)

19. An antibody-drug conjugate (ADC) of Formula (I):
Ab-(DL)ₙ (I);
wherein:
Ab is an antibody or an antigen-binding fragment thereof capable of binding to one or more of tumor-associated antigens or cell-surface receptors;
DL is the linker-payload according to any one of claims 1-8, 10-12, 16 where the payload is the drug unit, and the bioorthogonal group forms a covalent linkage with the antibody or the antigen-binding fragment thereof; and
n is a drug-to-antibody ratio (DAR) ranging from 1 to 20.

20. The ADC according to claim 19, wherein the antibody or antigen-binding fragment thereof is monospecific or multispecific.

21. The ADC according to claim 19, wherein the antibody or antigen-binding fragment thereof is bispecific to HER2 and TROP2, c-Met and HER3, EGFR and HER3, or EGFR and c-Met.

22. The ADC according to claim 19, wherein the ADC has enhanced tumor suppression efficacy and/or enhanced circulation stability.

23. (canceled)

24. A method for treating a disease, comprising administering to a subject in need thereof an effective amount of the ADC according to claim 19.

25. The method according to claim 24, wherein the disease is characterized by expressing CLDN18.2, B7-H₃, MUC-1, PD-1, PD-L1, CTLA-4, VEGF, BCMA, PSMA, CGRP, Tfr1, TNF alpha, tissue factor, folate receptor alpha, carcinoembryonic antigens (CEACAMs), c-MET, HER3, EGFR, HER2, TROP2, or Nectin-4.

26. The method according to claim 24, wherein the disease is a cancer selected from the group consisting of multiple myeloma, acute myeloid leukemia (AML), sarcoma, skin cancer, leukemia, lymphoma, brain cancer, glioblastoma, lung cancer, breast cancer, oral cancer, head-and-neck cancer, nasopharyngeal cancer, esophagus cancer, stomach cancer, liver cancer, bile duct cancer, gallbladder cancer, bladder cancer, pancreatic cancer, intestinal cancer, colorectal cancer, kidney cancer, cervix cancer, endometrial cancer, ovarian cancer, testicular cancer, buccal cancer, oropharyngeal cancer, laryngeal cancer, prostate cancer, thyroid cancer, and oral cancer.

27. (canceled)

28. (canceled)

29. (canceled)

30. (canceled)

31. A method for preparing an antibody-drug conjugate (ADC), comprising reacting an antibody or an antigen-binding fragment thereof with the linker-payload according to claim 1, and adding a co-solvent into a reaction mixture comprising the linker-payload and the antibody or the antigen-binding fragment thereof, wherein the payload is the drug unit.

32. (canceled)

33. The method according to claim 31, wherein the hydrophilic moiety comprises PEG and the co-solvent is propylene glycol.

34. The method according to claim 33, wherein the PEG has 1 to 48 ethylene glycol units.

35. The method according to claim 33, wherein the propylene glycol has a volume ratio ranging from 0-50%.
